(19) United States
(12) Patent Application Publication
(10) Pub. No.: US 2024/0225611 A1
(43) Pub. Date: Jul. 11, 2024

(54) TECHNIQUES OF MEASURING BRAIN INTRACRANIAL PRESSURE, INTRACRANIAL ELASTANCE, AND ARTERIAL BLOOD PRESSURE

(71) Applicant: Liminal Sciences, Inc., Guilford, CT (US)

(72) Inventors: Kamyar Firouzi, San Jose, CA (US); Mohammad Moghadamfalahi, San Jose, CA (US); Yichi Zhang, Sunnyvale, CA (US); Florian Dubost, Guilford, CT (US); Armin Moharrer, Guilford, CT (US); Amirreza Farnoosh, Guilford, CT (US)

(73) Assignee: Liminal Sciences, Inc., Guilford, CT (US)

(21) Appl. No.: 18/617,148

(22) Filed: Mar. 26, 2024

Related U.S. Application Data
(63) Continuation of application No. PCT/US2022/044949, filed on Sep. 27, 2022.
(60) Provisional application No. 63/249,011, filed on Sep. 27, 2021.

(51) Int. Cl. A61B 8/08; A61B 8/00; A61B 8/06; G16H 50/20

(52) U.S. Cl. CPC A61B 8/5223; A61B 8/06; G16H 50/20; A61B 8/4477

(57) ABSTRACT

Described herein are techniques for non-invasively measuring intracranial ICP in a subject's brain. Some embodiments use a physics guided machine learning model to determine measurements of various metrics (e.g., ICP, ABP, and/or ICE) of a subject's brain. The structure of the physics guided machine learning model may be based on a model of the brain (e.g., a hemodynamic or elastic model of the brain). The physics guided machine learning model may include various machine learning models (e.g., neural networks) representing different aspects of the brain's fluid dynamics and/or mechanics. The techniques may use acoustic measurement data (e.g., obtained using ultrasound) in conjunction with other information to generate inputs for the physics guided machine learning model. The inputs may be used to measurements of a metric for the subject's brain.

### RELATED APPLICATIONS

This application claims the benefit of priority under 35 U.S.C. § 120 as a continuation of PCT Patent Application No. PCT/US2022/044947, filed on Sep. 27, 2022, which claims the benefit under § 119(e) of U.S. Provisional Application No. 63/249,011 entitled “TECHNIQUES OF MEASURING BRAIN INTRACRANIAL PRESSURE, INTRACRANIAL ELASTANCE, AND ARTERIAL BLOOD PRESSURE”, filed on Sep. 27, 2021, the disclosures of which are incorporated herein by reference in their entirety.

### FIELD

This application relates generally to devices and techniques of measuring metrics of fluid dynamics and mechanics of a subject's brain including intracranial pressure (ICP), intracranial elastance (ICE), and/or arterial blood pressure (ABP). A measured metric may be used for diagnosis and treatment of condition in the subject.

### BACKGROUND

The brain comprises of cells (e.g., neurons and glia) and interstitial fluid that intertwine like different compartments of an engine. The brain includes a vast, fractal web of arteries, veins, and capillaries that circulate blood throughout brain tissue. Measurements of metrics of blood flow and mechanics of brain tissue are important for medical applications such as monitoring brain health, predicting seizures, and diagnosing diseases (e.g., strokes and swelling). The brain often reacts to adverse conditions (e.g., stroke, infection, aneurysm, concussion, etc.) by swelling. To diagnose and treat a subject's brain, clinicians use measurements of various brain mechanics such as ICP, volumetric cerebral blood flow (CBF), and/or ICE.

### SUMMARY

Described herein are techniques for non-invasively measuring intracranial ICP in a subject's brain. Some embodiments use a physics guided machine learning model to determine measurements of various metrics (e.g., ICP, arterial blood pressure (ABP), and/or ICE) of a subject's brain. The structure of the physics guided machine learning model may be based on a model of the brain (e.g., a hemodynamic or elastic model of the brain). The physics guided machine learning model may include various machine learning models (e.g., neural networks) representing different aspects of the brain's fluid dynamics and/or mechanics. The techniques may use acoustic measurement data (e.g., obtained using ultrasound) in conjunction with other information to generate inputs for the physics guided machine learning model. The inputs may be used for measurements of a metric for the subject's brain.

According to some embodiments, a method of determining intracranial pressure (ICP) of a subject's brain is provided. The method comprises: using at least one computer hardware processor to perform: obtaining acoustic measurement data obtained from measuring acoustic signals from the subject's brain; determining a cerebral blood flow velocity (CBFV) measurement of the subject's brain using the acoustic measurement data; obtaining an arterial blood pressure (ABP) measurement of the subject's brain; generating, using the CBFV measurement and the ABP measurement, input to a machine learning model trained to output an ICP measurement; and providing the input to the machine learning model to obtain an ICP measurement of the subject's brain.

According to some embodiments, an ICP measurement device is provided. The device comprises: one or more probes configured to obtain acoustic measurement data by detecting acoustic signals in a subject's brain; and at least one computer hardware processor configured to: determine a cerebral blood flow velocity (CBFV) measurement of the subject's brain using the acoustic measurement data; obtain an arterial blood pressure (ABP) measurement of the subject's brain; generate, using the CBFV measurement and the ABP measurement, input to a machine learning model trained to output an ICP measurement; and provide the input to the machine learning model to obtain an ICP measurement of the subject's brain.

According to some embodiments, a non-transitory computer-readable storage medium storing instructions is provided. The instructions, when executed by a processor, cause the processor to perform: obtaining acoustic measurement data obtained from measuring acoustic signals from the subject's brain; determining a cerebral blood flow velocity (CBFV) measurement of the subject's brain using the acoustic measurement data; obtaining an arterial blood pressure (ABP) measurement of the subject's brain; generating, using the CBFV measurement and the ABP measurement, input to a machine learning model trained to output an ICP measurement; and providing the input to the machine learning model to obtain an ICP measurement of the subject's brain.

According to some embodiments, a method of determining intracranial pressure (ICP) of a subject's brain is provided. The method comprises: obtaining acoustic measurement data from detecting acoustic signals from the subject's brain; determining an arterial blood pressure (ABP) measurement of the subject's brain using the acoustic measurement data; determining a cerebral blood flow (CBF) measurement using the acoustic measurement data; and determining an ICP measurement of the subject's brain using the CBF measurement and the ABP measurement, wherein determining the ICP measurement comprises using a physics guided machine learning model to obtain the ICP measurement of the subject's brain.

According to some embodiments, an ICP measurement device is provided. The device comprises: one or more probes configured to obtain acoustic measurement data by detecting acoustic signals in a subject's brain; and a computer hardware processor configured to: determine an arterial blood pressure (ABP) measurement of the subject's brain using the acoustic measurement data; determine a cerebral blood flow (CBF) measurement using the acoustic measurement data; and determine an ICP measurement of the subject's brain using the CBF measurement and the ABP measurement, wherein determining the ICP measurement comprises using a physics guided machine learning model to obtain the ICP measurement of the subject's brain.

According to some embodiments, a non-transitory computer-readable storage medium storing instructions is provided. The instructions, when executed by a processor, cause the processor to perform: determining an arterial blood pressure (ABP) measurement of the subject's brain using acoustic measurement data obtained from detecting acoustic signals from a subject's brain; determining a cerebral blood flow (CBF) measurement using the acoustic measurement data; and determining an ICP measurement of the subject's brain using the CBF measurement and the ABP measurement, wherein determining the ICP measurement comprises using a physics guided machine learning model to obtain the ICP measurement of the subject's brain.

According to some embodiments, a method of determining intracranial pressure (ICP) of a subject's brain is provided. The method comprises: obtaining acoustic measurement data and pulsatility data from detecting acoustic signals from the subject's brain; determining measure of brain perfusion using the acoustic measurement data and the pulsatility data; and determining an ICP measurement of the subject's brain using the measure of brain perfusion, wherein determining the ICP measurement comprises using a physics guided machine learning model to obtain the ICP measurement of the subject's brain.

According to some embodiments, an ICP measurement device is provided. The device comprises: one or more probes configured to obtain acoustic measurement data and pulsatility data by detecting acoustic signals from a subject's brain; and a processor configured to: determine a measure of brain perfusion using the acoustic measurement data and the pulsatility data; and determine an ICP measurement of the subject's brain using the measure of brain perfusion, wherein determining the ICP measurement comprises using a physics guided machine learning model to obtain the ICP measurement of the subject's brain.

According to some embodiments, a non-transitory computer-readable storage medium storing instructions is provided. The instructions, when executed by a processor, cause the processor to perform: determining a measure of brain perfusion using acoustic measurement data and pulsatility data obtained from detecting acoustic signals from a subject's brain; and determining an ICP measurement of the subject's brain using the measure of brain perfusion, wherein determining the ICP measurement comprises using a physics guided machine learning model to obtain the ICP measurement of the subject's brain.

According to some embodiments, a method of determining intracranial pressure (ICP) of a subject's brain is provided. The method comprises: obtaining acoustic measurement data obtained from measuring acoustic signals from the subject's brain; determining, using the acoustic measurement data, a ventricle deformation measurement of the subject's brain; and determining an ICP measurement of the subject's brain using the ventricle deformation measurement of the subject's brain, wherein determining the ICP measurement comprises using a physics guided machine learning model to obtain the ICP measurement of the subject's brain.

According to some embodiments, an ICP measurement device is provided. The device comprises: one or more probes configured to obtain acoustic measurement data from detecting acoustic signals in a subject's brain; and a processor configured to: determine, using the acoustic measurement data, a ventricle deformation measurement of the subject's brain; and determine an ICP measurement of the subject's brain using the ventricle deformation measurement of the subject's brain, wherein determining the ICP measurement comprises using a physics guided machine learning model to obtain the ICP measurement of the subject's brain.

According to some embodiments, a non-transitory computer-readable storage medium storing instructions is provided. The instructions, when executed by a processor, cause the processor to perform: determining, using acoustic measurement data obtained from measuring acoustic signals from a subject's brain, a ventricle deformation measurement of the subject's brain; and determining an ICP measurement of the subject's brain using the ventricle deformation measurement of the subject's brain, wherein determining the ICP measurement comprises using a physics guided machine learning model to obtain the ICP measurement of the subject's brain.

According to some embodiments, a method of determining arterial blood pressure (ABP) in a subject's brain is provided. The method comprises: obtaining acoustic measurement data obtained from detecting acoustic signals from the subject's brain; determining, using the acoustic measurement data, an arterial deformation measurement of the subject's brain; and determining an ABP measurement of the subject's brain using the arterial deformation measurement of the subject's brain, wherein determining the ABP measurement comprises using a physics guided machine learning model to obtain the ABP measurement of the subject's brain.

According to some embodiments, an ABP measurement device is provided. The device comprises: one or more probes configured to obtain acoustic measurement data from detecting acoustic signals from a subject's brain; and a processor configured to: determine, using the acoustic measurement data, an arterial deformation measurement of the subject's brain; and determine an ABP measurement of the subject's brain using the arterial deformation measurement of the subject's brain, wherein determining the ABP measurement comprises using a physics guided machine learning model to obtain the ABP measurement of the subject's brain.

According to some embodiments, a non-transitory computer-readable storage medium storing instructions is provided. The instructions, when executed by a processor, cause the processor to perform: determining, using acoustic measurement data obtained from detecting acoustic signals from a subject's brain, an arterial deformation measurement of the subject's brain; and determining an ABP measurement of the subject's brain using the arterial deformation measurement of the subject's brain, wherein determining the ABP measurement comprises using a physics guided machine learning model to obtain the ABP measurement of the subject's brain.

According to some embodiments, a method of determining arterial elastance in a subject's brain is provided. The method comprises: obtaining acoustic measurement data obtained from detecting acoustic signals from the subject's brain; determining, using the acoustic measurement data, an arterial deformation measurement of an artery in the subject's brain; and determining an arterial elastance measurement for the subject's brain using the arterial deformation measurement.

According to some embodiments, an arterial elastance measurement device is provided. The device comprises: one or more probes configured to obtain acoustic measurement data from detecting acoustic signals from a subject's brain; and a processor configured to: determine, using the acoustic measurement data, an arterial deformation measurement of an artery in the subject's brain; and determine an arterial elastance measurement for the subject's brain using the arterial deformation measurement.

According to some embodiments, a non-transitory computer-readable storage medium storing instructions is provided. The instructions, when executed by a processor, cause the processor to perform: determining, using acoustic measurement data obtained from detecting acoustic signals from a subject's brain, an arterial deformation measurement of an artery in the subject's brain; and determining an arterial elastance measurement for the subject's brain using the arterial deformation measurement.

According to some embodiments, a method of determining intracranial elastance (ICE) of a subject's brain is provided. The method comprises: obtaining acoustic measurement data obtained from detecting acoustic signals from the subject's brain; determining, using the acoustic measurement data, a measurement of movement of one or more tissue areas in the subject's brain; and determining an intracranial elastance measurement of the subject's brain based on the measurement of movement of the one or more tissue areas in the subject's brain.

According to some embodiments, a device for measuring intracranial elastance in a subject's brain is provided. The device comprises: one or more probes configured to obtain acoustic measurement data from detecting acoustic signals from a subject's brain; and a processor configured to: determine, using the acoustic measurement data, a measurement of movement of one or more tissue areas in the subject's brain; and determine an intracranial elastance measurement of the subject's brain based on the measurement of movement of the one or more tissue areas in the subject's brain.

According to some embodiments, a non-transitory computer-readable storage medium storing instructions is provided. The instructions, when executed by a processor, cause the processor to perform: determining, using acoustic measurement data obtained from detecting acoustic signals from a subject's brain, a measurement of movement of one or more tissue areas in the subject's brain; and determining an intracranial elastance measurement of the subject's brain based on the measurement of movement of the one or more tissue areas in the subject's brain.

### BRIEF DESCRIPTION OF THE DRAWINGS

Various aspects and embodiments will be described herein with reference to the following figures. It should be appreciated that the figures are not necessarily drawn to scale. Items appearing in multiple figures are indicated by the same or a similar reference number in all the figures in which they appear.

FIGS. 1A-C shows a collection of arteries at the base of the brain called the Circle of Willis.

FIG. 2 shows an overview of the brain's ventricles.

FIG. 3 shows a graph of an intracranial compliance (ICC) curve, according to some embodiments of the technology described herein.

FIG. 4 shows a graph illustrating of cerebral autoregulation capacity of a brain, according to some embodiments of the technology described herein.

FIG. 5 shows an example circuit modelling hemodynamics of the brain, according to some embodiments of the technology described herein.

FIG. 6 shows an example hemodynamic model of the brain, according to some embodiments of the technology described herein.

FIG. 7 shows an example graph of CBF, ICP, and ABP waveforms, according to some embodiments of the technology described herein.

FIG. 8 shows an example graph of an ICP waveform in a healthy subject, according to some embodiments of the technology described herein.

FIG. 9 shows a graph of ICP vs. intracranial volume, according to some embodiments of the technology described herein.

FIG. 10A shows an example acousto-encephalography (AEG) device which may be used to implement some embodiments of the technology described herein.

FIG. 10B shows an example placement of AEG probes of the AEG device of FIG. 10A on a subject's head, according to some embodiments of the technology described herein.

FIG. 10C shows a system in which the AEG device of FIG. 10A may be used, according to some embodiments of the technology described herein.

FIG. 10D shows an example architecture of the AEG device of FIG. 10A, according to some embodiments of the technology described herein.

FIG. 11 shows an example ICP measurement system, according to some embodiments of the technology described herein.

FIG. 12A shows an example physics guided machine learning model that may be used by to obtain an ICP measurement, according to some embodiments of the technology described herein.

FIG. 12B shows another example machine learning model that may be used to obtain an ICP measurement, according to some embodiments of the technology described herein.

FIG. 12C shows another example machine learning model that may be used to obtain an ICP measurement, according to some embodiments of the technology described herein.

FIG. 12D shows another example machine learning model that may be used to obtain an ICP measurement, according to some embodiments of the technology described herein.

FIG. 12E shows another example machine learning model that may be used to obtain an ICP measurement, according to some embodiments of the technology described herein.

FIG. 13 shows an example process that may be performed by the ICP measurement system of FIG. 11 to determine an ICP measurement of a subject's brain, according to some embodiments of the technology described herein.

FIG. 14 shows an example joint ICP/ABP measurement system, according to some embodiments of the technology described herein.

FIG. 15A shows an example ML model that may be used to obtain an ABP measurement, according to some embodiments of the technology described herein.

FIG. 15B shows another example ML model that may be used to obtain an ABP measurement, according to some embodiments of the technology described herein.

FIG. 15C shows another example ML model that may be used to obtain an ABP measurement, according to some embodiments of the technology described herein.

FIG. 16 shows an example process that may be performed by the ICP/ABP measurement system of FIG. 14 to determine ABP and ICP of a subject's brain, according to some embodiments of the technology described herein.

FIG. 17 shows an example ICP measurement system, according to some embodiments of the technology described herein.

FIG. 18 shows an example process that may be performed by the ICP measurement system of FIG. 17 to determine an ICP measurement of a subject's brain, according to some embodiments of the technology described herein.

FIG. 19 shows an example ICP measurement system, according to some embodiments of the technology described herein.

FIG. 20 shows an example process that may be performed by the ICP measurement system of FIG. 19 to determine an ICP measurement of a subject's brain, according to some embodiments of the technology described herein.

FIG. 21 shows an example ABP measurement system, according to some embodiments of the technology described herein.

FIG. 22 shows an example process that may be performed by the ABP measurement system of FIG. 21 to determine an ABP measurement of a subject's brain, according to some embodiments of the technology described herein.

FIG. 23 shows an example arterial elastance measurement system, according to some embodiments of the technology described herein.

FIG. 24 shows an example process that may be performed by the arterial elastance measurement system of FIG. 23 to determine arterial elastance of a subject's brain, according to some embodiments of the technology described herein.

FIG. 25 shows an example brain elasticity measurement system, according to some embodiments of the technology described herein.

FIG. 26 shows an example process that may be performed by the brain elasticity measurement system of FIG. 25 to determine elasticity of a subject's brain, according to some embodiments of the technology described herein.

FIG. 27 shows an example machine learning model for segmentation of a CBFV signal envelope and heartbeat boundaries, according to some embodiments of the technology described herein.

FIG. 28A shows a plot of an example CBFV signal, according to some embodiments of the technology described herein.

FIG. 28B shows a plot showing the CBFV signal of FIG. 28A with the signal's envelope segmented, according to some embodiments of the technology described herein.

FIG. 29 illustrates segmentation of a CBFV signal of a heartbeat signal, according to some embodiments of the technology described herein.

FIG. 30 shows a block diagram for a wearable device for autonomous beam steering, according to some embodiments of the technology described herein.

FIG. 31 shows example beamforming techniques, according to some embodiments of the technology described herein.

FIG. 32A shows a flow diagram for a method for autonomous beam-steering, according to some embodiments of the technology described herein.

FIG. 32B shows a flow diagram for a method for detecting, localizing, and/or segmenting a ventricle, according to some embodiments of the technology described herein.

FIG. 32C shows a flow diagram for detecting, localizing, and/or segmenting the circle of Willis, according to some embodiments of the technology described herein.

FIG. 32D shows a flow diagram for a method for localizing a blood vessel, according to some embodiments of the technology described herein.

FIG. 32E shows a flow diagram for a method of locking onto a region of interest, according to some embodiments of the technology described herein.

FIG. 32F shows a flow diagram for a method for estimating a shift due to a drift in hardware, according to some embodiments of the technology described herein.

FIG. 32G shows a flow diagram for a method for estimating a shift associated with the detected signal, according to some embodiments of the technology described herein.

FIG. 33 shows diagrams for example beam-steering techniques, according to some embodiments of the technology described herein.

FIG. 34 shows example data processing pipelines, according to some embodiments of the technology described herein.

FIG. 35A shows an example diagram of the Deep Neural Network (DNN) framework used for estimating the relative positions of two regions in the same image, according to some embodiments of the technology described herein.

FIG. 35B shows an example algorithm for template extraction, according to some embodiments of the technology described herein.

FIG. 36 shows a block diagram for reinforcement-learning based guidance for target locking, according to some embodiments of the technology described herein.

FIG. 37 is a block diagram showing an example algorithm for tracking hardware drifts, according to some embodiments of the technology described herein.

FIG. 38 is a block diagram showing an example algorithm for tracking signal shifts, according to some embodiments of the technology described herein.

FIG. 39A shows an example diagram of ventricles in the brain, according to some embodiments of the technology described herein.

FIG. 39B shows a flow diagram of an example system for ventricle detection and segmentation, according to some embodiments of the technology described herein.

FIG. 39C shows an example process and data for brain ventricle segmentation, according to some embodiments of the technology described herein.

FIG. 40 shows a flow diagram of an example algorithm for circle of Willis segmentation, according to some embodiments of the technology described herein.

FIG. 41A shows a flow diagram for an example algorithm for estimating the vessel diameter and/or curve, according to some embodiments of the technology described herein.

FIG. 41B shows an example vessel diameter estimation, according to some embodiments of the technology described herein.

FIG. 41C shows an example segmentation of a vessel, according to some embodiments of the technology described herein.

FIG. 42 shows an illustrative flow diagram for a process for constructing and deploying a machine learning algorithm, according to some embodiments of the technology described herein.

FIG. 43 shows a convolutional neural network that may be used in conjunction with an AEG device, in accordance with some embodiments of the technology described herein.

FIG. 44 shows an illustrative process for determining a measure of brain tissue motion in a brain, according to some embodiments of the technology described herein.

FIG. 45 shows an illustrative process for determining a measure of brain tissue motion in a brain via spatiotemporal filtering, according to some embodiments of the technology described herein.

FIG. 46 shows an illustrative example of unwrapping a three-dimensional image stack into a two-dimensional matrix, according to some embodiments of the technology described herein.

FIG. 47 shows an illustration of the power spectra of temporal singular vectors in decreasing singular value order, according to some embodiments of the technology described herein.

FIG. 48 shows examples of singular value decomposition filtering on a sequence of B-mode images of a patient's brain, according to some embodiments of the technology described herein.

FIG. 49 shows an illustrative process for determining a measure of brain tissue motion in a brain via signal decomposition, according to some embodiments of the technology described herein.

FIG. 50 shows an example flow diagram of kernel principal component analysis, according to some embodiments of the technology described herein.

FIG. 51 shows example images of extracted components from kernel principal component analysis, according to some embodiments of the technology described herein.

FIG. 52A shows an example flow diagram of independent component analysis, according to some embodiments of the technology described herein.

FIG. 52B shows example images of extracted components from independent component analysis, according to some embodiments of the technology described herein.

FIG. 53 shows an illustrative process for determining a measure of brain tissue motion in a brain via tissue tracking, according to some embodiments of the technology described herein.

FIG. 54 shows an example flow diagram of tissue tracking using finite differences, according to some embodiments of the technology described herein.

FIG. 55 shows an example flow diagram of ventricle edge tracking for brain beat extraction, according to some embodiments of the technology described herein.

FIG. 56 shows an example of ventricle wall detection, according to some embodiments of the technology described herein.

FIG. 57 show examples of sample beat signals extracted from different regions of the ventricle, according to some embodiments of the technology described herein.

FIG. 58 shows an illustrative process for determining a measure of brain tissue motion in a brain via spectral clustering, according to some embodiments of the technology described herein.

FIG. 59 shows an example flow diagram of spatiotemporal clustering, according to some embodiments of the technology described herein.

FIG. 60 shows example cerebral blood flow, intracranial pressure, and pulsatility waveforms, according to some embodiments of the technology described herein.

FIG. 61 is a block diagram of an example computer system, according to some embodiments of the technology described herein.

### DETAILED DESCRIPTION

Described herein are techniques of non-invasively measuring metrics of fluid mechanics of a subject's brain. The metrics include intracranial pressure (ICP), arterial blood pressure (ABP), arterial elastance, and intracranial elastance (ICE).

ICP and/or ICE may be monitored in order to diagnose and treat various conditions in a brain. For example, ICP and/or ICE may be used in diagnosing and treating traumatic brain injury, epilepsy, intracerebral hemorrhage, subarachnoid hemorrhage, hydrocephalus, malignant infarction, cerebral edema, central nervous system (CNS), epilepsy, and/or infections hepatic encephalopathy. ICP may be monitored by a clinician and used to determine treatment of a condition in a subject's brain. One conventional technique of monitoring ICP is to use an intraventricular catheter connected to an external pressure transducer. The catheter is placed into one of the brain's ventricles through a burr hole. Other conventional techniques of monitoring ICP include use of intraparenchymal monitors, subdural devices, epidural devices, and/or a lumbar puncture.

Conventional invasive techniques of monitoring ICP have risk of several complications. For example, conventional techniques have a risk of infection, obstruction, difficulty in placement, malposition, disconnection, and/or device failure. Conventional non-invasive techniques of monitoring ICP (e.g., transcranial doppler (TCD) ultrasound, near infrared (IR) spectroscopy, magnetic resonance imaging (MRI), computed tomography (CT), etc.) lack accuracy of the invasive techniques. For example, conventional non-invasive techniques lack accuracy in pressure numeric values as well as in time-wave forms of ICP.

One conventional technique for monitoring the brain is transcranial ultrasound or transcranial doppler (TCD). Conventional TCD and ultrasound techniques rely on high-end ultrasound scanners or a dedicated TCD system. TCD devices are noninvasive bedside equipment used for measuring cerebral vasculature and blood flow velocity in the brain's blood vessels or flow velocity in intracranial arteries. TCD takes advantage of natural thin areas in the skull such as the temporal window (near the pterion) and Posterior below the foramen magnum to couple ultrasound into the brain. TCD devices are used for diagnosis of conditions such as stenosis, emboli, hemorrhage, sickle cell disease, ischemic cerebrovascular disease, vasospasm, and cerebral circulatory arrest. For measuring ICP using TCD, blood flow metrics such as systolic and diastolic wavers, pulsatility index (PI) and the Lindegaard ratio (LR) in major arteries at the base of the skull (the Circle of Willis) are used in statistical or physiological models to extract ICP. TCD is not alone reliable for ICP or brain monitoring, and high end ultrasound scanners are typically limited in the frame rate, bulky, and expensive. Moreover, TCD systems are limited to only measuring blood velocity in the vasculature at the base of the brain and rely on single-element transducer technology. As such, techniques that use TCD have a high level of uncertainty and questionable accuracy, as the location and angle with respect to vessels are difficult to determine accurately. Further, TCD systems may be difficult to use and require an operator to be trained on how to place a probe and to identify the correct locations of vessels (e.g., around the Circle of Willis). Some conventional TCD systems provide a robotic arm and headset that can automatically adjust a probe to maintain a high quality signal. However, this feature is expensive and not realistic for broad application.

To address the above-described shortcomings of conventional systems and techniques, techniques described herein non-invasively determine ICP of a subject's brain. Some embodiments described herein use a machine learning model (e.g., a neural network) with has a structure that models the brain to determine ICP of a subject's brain. In some embodiments, the machine learning model may be a physics guided machine learning model. For example, the physics guided machine learning model structure may be based on a hemodynamic or elastic model of the brain. The techniques use acoustic measurement data (e.g., obtained using ultrasound) in combination with other information to generate input for the machine learning model. In some embodiments, the techniques use acoustic measurement data in combination with a measurement of CBF to generate input for the machine learning model. The system provides the input to the machine learning model to obtain an output indicating an ICP measurement of a subject's brain.

Another metric of fluid mechanics of the brain is arterial elastance. The arterial elastance of an artery may indicate elastance of brain tissue neighboring the artery. Moreover, the arterial elastance may provide a measure of cardiovascular health. Some embodiments of techniques described herein use acoustic measurement data (e.g., obtained using ultrasound) to determine a measurement of arterial elastance of an artery in a subject's brain. Another metric of fluid mechanics of the brain is ICE. Some embodiments of techniques described herein use pulsatility data indicating movement in a subject's brain to determine an intracranial elastance measurement of the brain.

### Brain Biology

The brain is composed of the cerebrum, cerebellum, and brainstem. The cerebrum is the largest part of the brain and is composed of right and left hemispheres. The cerebrum performs higher functions such as interpreting sensory signals (e.g., touch, vision, and hearing), speech, reasoning, emotions, learning, and fine control of movement. The cerebellum is located under the cerebrum. The cerebellum coordinates muscle movements, maintains posture, and maintains balance. The brainstem acts as a relay center connecting the cerebrum and cerebellum to the spinal cord. It performs many automatic functions such as breathing, heart rate, body temperature, wake and sleep cycles, digestion, sneezing, coughing, vomiting, and swallowing.

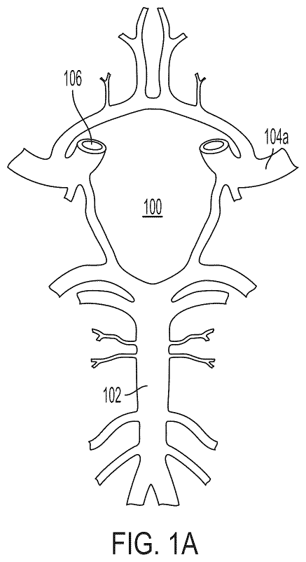

The Circle of Willis is a collection of arteries at the base of the brain that provides brain its blood supply. FIGS. 1A-1C show the Circle of Willis 100 and its location in the brain 108. The arteries included in the Circle of Willis 100 comprise the basilar artery 102, the middle cerebral artery 104a, anterior cerebral artery 104b, posterior cerebral artery 104c, the internal carotid artery 106, the vertebral artery 110, the posterior communicating artery 112a, and the anterior communicating artery 112b.

The Circle of Willis provides the blood supply to the brain. In general terms, the Circle of Willis connects two arterial sources together to form the arterial circle shown in FIGS. 1A-1C, which then supplies oxygenated blood to over 80% of the cerebrum. The structure encircles the middle area of the brain, including the stalk of the pituitary gland and other important structures. The two carotid arteries 106 supply blood to the brain through the neck and lead directly to the Circle of Willis 100. Each carotid artery branches into an internal and external carotid artery. The internal carotid artery then branches into the cerebral arteries 104a-c. This structure allows all of the blood from the two internal carotid arteries to pass through the Circle of Willis. The internal carotid arteries branch off from here into smaller arteries, which deliver much of the brain's blood supply.

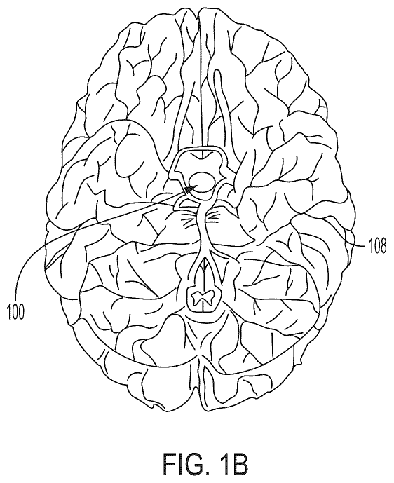

FIG. 2 shows an overview of the brain's ventricles. The brain ventricles are four internal cavities that contain cerebrospinal fluid (CSF). Ventricles are critically important to the normal functioning of the central nervous system. Infection (such as meningitis), bleeding or blockage can change the characteristics of the CSF. Brain ventricles' shape can be very useful in diagnosing various conditions such as intraventricular hemorrhage and intracranial hypertension. CSF flows within and around the brain and spinal cord to help cushion it from injury. This circulating fluid is constantly being absorbed and replenished. There are two ventricles deep within the cerebral hemispheres called the lateral ventricles 204. They both connect with the third ventricle 208 through a separate opening called the Foramen of Monro 202. The third ventricle 208 connects with the fourth ventricle 210 through a long narrow tube called the aqueduct of Sylvius 206. From the fourth ventricle 210, CSF flows into the subarachnoid space where it bathes and cushions the brain 108. CSF is recycled (or absorbed) by special structures in the superior sagittal sinus called arachnoid villi. A balance is maintained between the amount of CSF that is absorbed and the amount that is produced. A disruption or blockage in the system can cause a buildup of CSF, which can cause enlargement of the ventricles (hydrocephalus) or cause a collection of fluid in the spinal cord (syringomyelia).

The human brain is a soft and complex material that is in constant motion due to underlying physiological dynamics. During each cardiac cycle (“heartbeat”), periodic variations in arterial blood pressure are transmitted along vasculatures resulting in subtle and relatively localized deformation and motion of brain tissue. Further, maintenance of adequate blood flow to the brain (“cerebral blood flow” (CBF)) is critical for normal brain function. Variations of cerebral blood flow are associated with various different conditions. Cerebral blood flow changes throughout a cardiac cycle. A systolic increase in blood pressure over the cardiac cycle causes regular variations in blood flow into and throughout the brain that are synchronized with the heartbeat. The changes in blood flow are transferred to brain tissue and other fluids in the brain (e.g., cerebrospinal fluid and/or blood).

Because the brain is contained in a fixed volume of the skull, the changes in blood flow result in motion of the brain tissue and fluid called brain “pulsatility”. Pulsatility exists in all three major components of the brain: brain tissue, blood, and cerebral fluid. The study of the brain's pulsatility is important in diagnosis and treatment of various conditions of the brain including hydrocephalus and traumatic brain injury where large changes in ICP and in biomechanical properties of the brain cause significant changes pulsatility.

Brain tissue is soft matter with hyper-elastic incompressible material behavior. Thus, the brain can experience deformations or strain while maintaining a constant volume. The Monroe Kellie doctrine describes that because the brain is incompressible, when the skull is intact, the sum of the volume of the brain tissue, cerebrospinal fluid (CSF), and intracranial blood is constant. Incompressibility leads to build-up of background steady stress or pressure inside the brain. Changes in ICP are a steady stress which have acoustoelastic effects on the brain. An acoustoelastic effect is how wave velocities of an elastic material change when subjected to stress.

ICP is defined as the pressure inside the skull. Thus, ICP is the pressure inside the brain tissue and the cerebrospinal fluid (CSF). Typically, ICP is usually considered to be 5-15 mmHg in a healthy supine adult, 3-7 mmHg in a healthy supine child, and 1.5-6 mmHg in a supine healthy infant. An ICP greater than 20 mmHg is considered elevated and may be a cause of irreversible brain injury or death. Intracranial hypertension is defined as ICP greater than 20 mmHg sustained for greater than 5 minutes. An acute increase in ICP may begin to manifest clinical symptoms requiring intervention. Isolated intracranial hypertension typically does not decrease the level of consciousness until ICP is greater than 40 mmHg. However, a shift in brain structure at a lower ICP may still result in a coma.

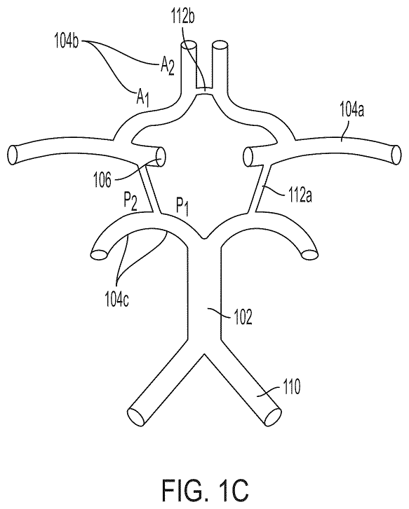

Intracranial compliance (ICC) is the brain's capacity to auto-compensate for changes in intracranial volume. ICC may be measured as the change in volume per unit change in pressure. ICE is the inverse of ICC. ICC and ICE indicate the ability of the intracranial compartment to accommodate an increase in volume without a large increase in ICP. FIG. 3 shows a graph 300 of an ICC curve, according to some embodiments of the technology described herein. The graph 300 shows a plot of intracranial volume vs. ICP. The ICE is given by the slope of the curve shown in the graph 300. Under normal conditions, a brain has high intracranial compliance 302 (i.e., a relatively low change in volume per unit change in pressure) by displacement of CSF and/or blood. As the change in volume increases, the brain has a decreasing level of compliance 304. The ICC curve enters a region of minimal compliance 306 where there is an increased risk of cerebral ischemia and herniation. Eventually the brain enters a region 208 in which there is a collapse of the cerebral microvasculature.

### Hemodynamics Brain Model

A hemodynamic model of the brain may model fluid mechanics of the brain's three main constituents, brain tissue, blood, and CSF. The hemodynamic model may include one or more lumped parameters. A lumped parameter models a complex structure of a vascular bed as a single tube that has the “lumped” properties of the vascular bed as a whole. The lumped parameter model is a variant of the “black box” concept, in which a complex system is modeled by an imaginary box, where the relationship between inputs and outputs of the box are examined to learn about the system inside the black box. The lumped parameter concept assumes that flow through a complex vascular bed can be replaced by the flow in a single tube with representative properties.

Three important factors that affect hemodynamics are flow resistance, compliance of a tube wall, and fluid inertia. One form of resistance to flow in a tube is due to viscous friction at the interface between the fluid and the tube wall. This type of resistance is present both when the flow is steady or oscillatory, and always dissipates energy. This form of resistance may be modeled by equation 1 below.

$$q = \frac{Δ p}{R} \qquad Equation ( 1 )$$

In equation 1 above, R is the resistance, q is the flow, and AP is the change in pressure.

Another effect is fluid inertia (or inductance). Acceleration in fluid flow may occur in one of two ways: space or in time. Acceleration in space may occur when the area available to a stream of fluid is decreasing and, as a result, the velocity of the fluid flow increases to continue flowing through the reduced area assuming that the flow is incompressible (i.e., that the fluid density is not changing). Accordingly, the fluid is in a state of acceleration as the area is reduced. Acceleration in time may occur when the velocity distribution changes over time. This may occur when pressure driving the flow is not constant in time. For example, in pulsatile blood flow, the pressure driving the blood changes in an oscillatory manner. Due to the oscillation of blood pressure, the flow at points in a flow field changes over time. As modeled by equation 2 below, the required pressure is proportional to the rate of change of flow rate.

$$Δ {P}_{l} = L \frac{dq}{dt} \qquad Equation ( 2 )$$

In equation 2 above, ΔPₗ is the change in pressure, dq/dt is the change of flow rate, and L represents compliance of the brain.

Another effect is compliance (or capacitance) of the tube wall. A tube in which the walls are rigid offers a fixed amount of space within it, hence the volume of fluid therein is also fixed, assuming the fluid is incompressible. There is thus only one flow rate through the tube, which may vary at different points in time depending on the applied pressure gradient. In contrast, when flow is occurring in a nonrigid tube, two new factors affect flow: (1) a change in volume of the fluid known as tube compliance; and (2) a local change of pressure within the tube may cause a local change in volume of fluid which propagates as a wave crest or value down the tube at a finite speed known as pulse wave velocity (PWV).

As a result of tube compliance, there is a change in total volume of fluid contained within a tube. The total change in volume of fluid contained within the tube may occurs in a transient state which may be approximated by an analogy to capacitors in which a change in pressure leads to a change in flow as a consequence of conservation of momentum. Equation 3 below captures the transient effect of compliance.

$${q}_{c} = C \frac{d ( Δ p )}{dt} \qquad Equation ( 3 )$$

In equation 3 above, qc represents the flow,

$$\frac{d ( Δ p )}{dt}$$

represents change in pressure, and C represents cerebral autoregulatory mechanism of the brain. Cerebrovascular autoregulation maintains a constant cerebral perfusion pressure (CPP) while ICP is changing. A pressure reactivity index (PRx), which is the time-averaged correlation coefficient between ICP and mean arterial blood pressure, may be used as an indicator of cerebrovascular autoregulation of the brain. A positive PRx indicates an impaired autoregulatory capacity of the brain, while a negative PRx indicates a normal autoregulatory capacity.

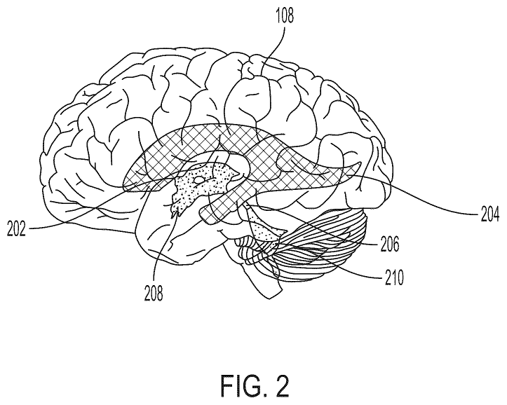

FIG. 4 shows a graph 400 illustrating of cerebral autoregulation capacity of a brain, according to some embodiments of the technology described herein. The graph 400 plots CBF vs. cerebral perfusion pressure (CPP). In the region 402 when the CPP is between approximately 50 mmHg to 150 mmHg, the brain maintains a constant CBF of approximately 50 mL/100 g/min. In the region 406, when the CPP is less than approximately 50 mmHg, the brain may have hypoperfusion-ischemia. In the region 404, when the CPP is greater than approximately 150 mmHg, the brain may have hyperfusion-hyperemia and edema.

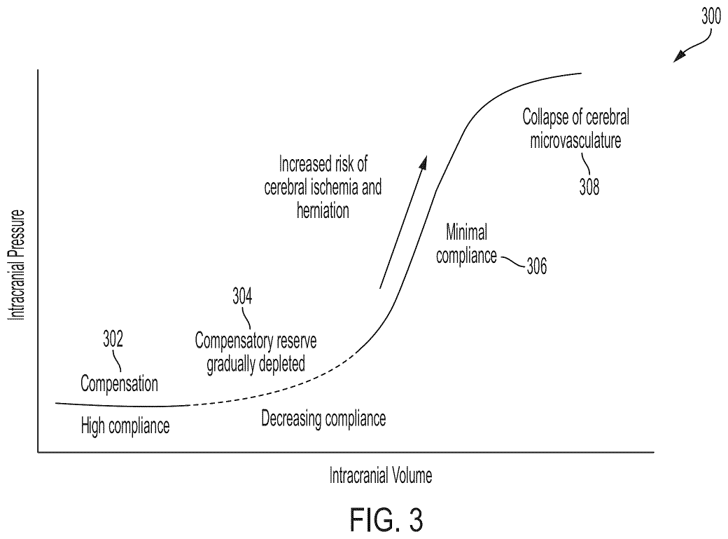

The forces described by equations 1, 2, and 3 above may be used to represent a hemodynamics fluid system that models the brain. In some embodiments, the hemodynamics fluid system may be a circuit that models the brain. FIG. 5 shows an example circuit 500 modelling hemodynamics of the brain, according to some embodiments of the technology described herein. The circuit 500 is an LRC circuit including a resistor 502 representing flow resistance, a capacitor 504 representing compliance (e.g., by cerebral autoregulation), and an inductor 506 representing fluid inertia. The hemodynamic system may be described by equation 4 below.

$$q ( t ) + \frac{L}{R} \frac{dq ( t )}{dt} + LC \frac{{d}^{2} q ( t )}{{dt}^{2}} = \frac{x ( t )}{R} + C \frac{dx ( t )}{dt} \qquad Equation ( 4 )$$

The CPP of the brain may be defined in the circuit by equation 5 below.

$$CPP = ABP - ICP = {p}_{a} ( t ) - {p}_{ic} ( t ) = x ( t ) \qquad Equation ( 5 )$$

Equations 4 and 5 above may thus provide a hemodynamic system modelling the brain.

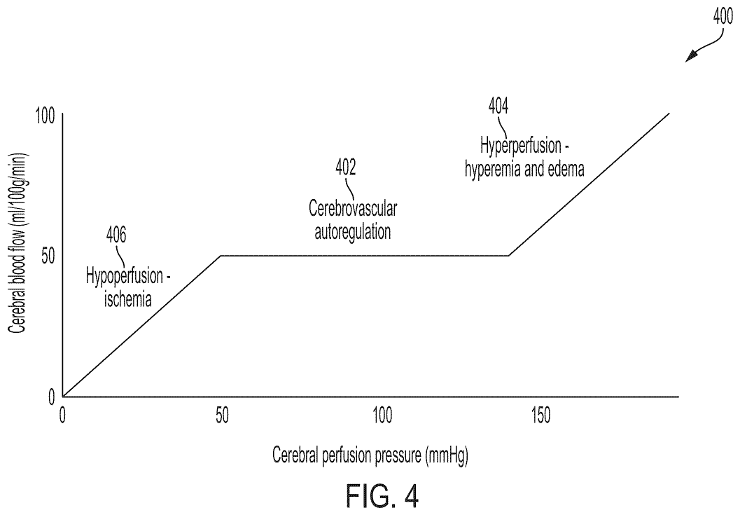

FIG. 6 shows an example hemodynamic model 600 of the brain, according to some embodiments of the technology described herein. The hemodynamic model 600 illustrates how blood flows throughout the brain. The model 600 includes a layer of CSF 604 surrounding the brain tissue. There is a CBF 602 into the brain that enters through the arteries 606, then goes through arterioles 608, then through capillaries 610, venules 612, and then into a vein 614.

### Pathophysiology of ICP

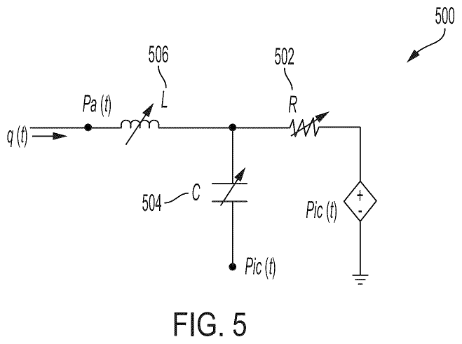

In the human body, the heart functions as an endogenous mechanical driver that induces motion over a cardiac cycle in the brain. The motion leads to transient changes in blood flow and pressure in the brain. FIG. 7 shows an example graphs 700 of CBF, ICP, and ABP waveforms. Graph 702 shows a waveform of ICP, graph 704 shows a waveform of CBFV, and graph 706 shows a waveform of ABP. The waveforms are synchronous with an arterial pulse resulting from pumping of the heart.

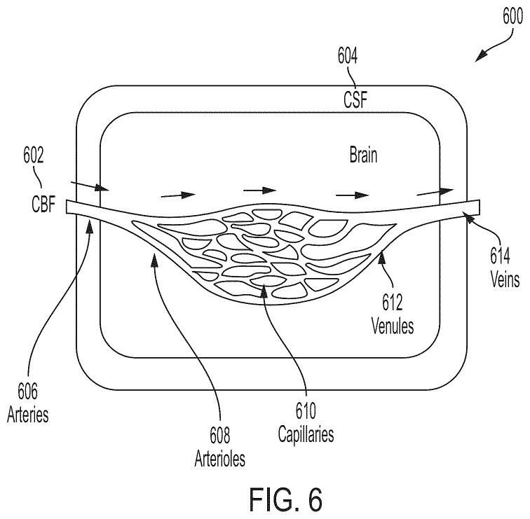

The ICP of a healthy subject's brain has a trifid waveform. FIG. 8 shows an example graph 800 of an ICP waveform in a healthy subject. Each wave of the ICP waveform shown in graph 800 has three peaks. The waveform of graph 800 includes a first peak 802, a second peak 804, and a third peak 806. The peaks may correlate to ABP. Generally, the ICP waveform has an amplitude less than 4 mmHg, or 10-30% of the mean ICP. The wave of first peak 802 (also referred to as the “percussion wave”) correlates with the arterial pulse transmitted through the choroid plexus into the CSF. The wave of the second peak 804 (also referred to as “reflection”) corresponds to the arterial pulse wave bouncing off the springy brain parenchyma. The wave of the third peak 806 (also referred to as “dicrotic wave”) corresponds to the closure of the aortic valve, which makes the trough prior to the third peak 806 the dicrotic notch.

Changes in ICP waveforms may correlate with different brain conditions. For example, increasing amplitude of all waveforms indicates rising ICP. In another example, decreasing amplitude of the waveform of the first peak 802 indicates decreased cerebral perfusion. In another example, increasing amplitude of the wave of the second peak 804 indicates decreased cerebral compliance. In another example, plateau waves suggest intact cerebral blood flow autoregulation. These changes may eventually manifest as low frequency issue strain which, due to its dynamic nature, leads to different temporal patterns of pulsatility in brain tissue and pulsatility in cerebral blood flow. A visualization of the ICP waveform may also be important in determining intracranial compliance, which may guide ICP therapies.

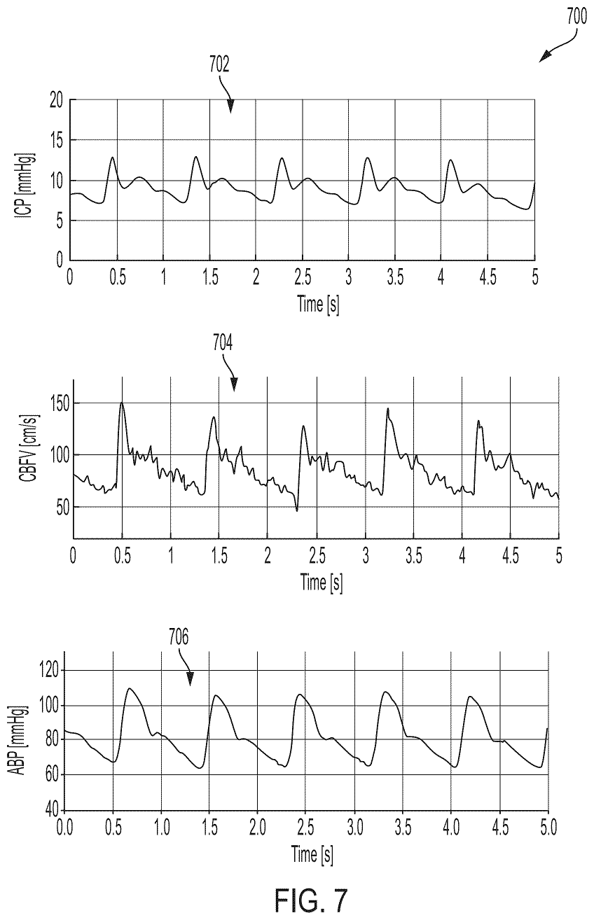

In a healthy brain, the waveforms of peaks 802, 804, 806 follow a decreasing trend with a baseline value around 5-15 mmHg. In an injured brain, the amplitude of the waveforms 802, 804, 806 may follow a different pattern with baseline value shifted to over 20-25 mmHg. FIG. 9 shows a graph 900 of ICP vs. intracranial volume. As can be seen in graph 900, when there is a normal ICP waveform 902, the brain has greater compliance. By contrast, when there is a non-compliant ICP waveform 904, the brain has lower compliance.

### Example Acousto-Encephalography (AEG) Device

Some embodiments may be implemented using an AEG device (e.g., to measure acoustic signals in a subject's brain). In some embodiments, the AEG device may be a smart, noninvasive, transcranial ultrasound platform for measuring brain vitals (e.g., pulse, pressure, flow, softness) that can diagnose and monitor brain conditions and disorders. The AEG device improves over conventional neuromonitoring devices because of features, including but not limited to, being easy-to-use (AEG does not require prior training or a high degree of user intervention) and being smart (AEG is empowered by an AI engine that account for the human factor and as such minimize any errors). It also improves the reliability or accuracy of the measurements. This expands its use cases beyond what is possible with conventional brain monitoring devices. For example, with portable/wearable stick-on probes, the AEG device can be used for both continuous monitoring and/or rapid screening.

In some embodiments, the AEG device is capable of intelligently steering ultrasound beams in the brain in three dimensions (3D) using techniques described herein. With 3D beam-steering, AEG can scan and interrogate various regions in the cranium, and assisted with AI, it can identify an ideal region of interest (ROI). AEG then locks onto the ROI and conducts measurements, while the AI component keeps correcting for movements and drifts from the target. The AEG device operates through three phases: 1-Lock, 2-Sense, 3-Track.

During the Lock phase, AEG, at a relatively low repetition rate, “scans” the cranium to identify and lock onto the ROI, by using AI-based smart beam-steering that utilizes progressive beam-steering to narrow down the field-of-view to a desired target region, by exploiting a combination of various anatomical landmarks and motion in different compartments. Different types of ROIs may be determined by the “presets” in a web/mobile App such as different arteries or beating at a specific depth in the brain. The ROI can be a single point, relatively small volume, or multiple points/small volumes at one time. The latter is a unique capability that can probe propagating phenomena in the brain, such as the pulse-wave-velocity (PWV).

During the Sense phase, the AEG device measures ultrasound footprints of different brain compartments using different pulsation protocols at a much higher repetition rate, to support pulsatile mode, to take the pulse of the brain. The AEG device can also measure continuous wave (CW)-, pulse wave (PW)-, and motion (M)-modes to look at blood flow and motion at select depths.

During the Track phase, the AEG device utilizes a feedback mechanism to evaluate the quality of the measurements. Once the device detects misalignment and misdetection, it goes back to state 1, to properly re-lock onto the target region.

In some embodiments, the AEG device includes core modes of measurements and functionalities, including ability to take the pulse of the brain, ability to measure pulse wave velocity (PWV) by probing multiple ROIs at one time, and ability to measure other ultrasound modes in the brain, including B-mode (brightness-mode) and C-mode (cross-section mode), blood velocity using CW (continuous-wave) and PW (pulse-wave) doppler, color flow imaging (CFI), PD (power-doppler), M-mode (motion-mode), and blood flow (volume rate).

In some aspects, the AEG device can include a hub and multiple probes to access different brain compartments such as temporal and suboccipital from various points over the head. The hub hosts the main hardware, e.g., analog, mixed, and/or digital electronics. The AEG device can be wearable, portable or an implantable (i.e., under the scalp or skull). In a fully wearable form, the AEG device can also be one or several connected small patch probes. Alternatively, the AEG device can be integrated into a helmet or cap. The AEG device can be wirelessly charged or be wired. It can transfer data wired or wirelessly to a host that can be worn (such as a watch or smart phone), bedside/portable (such as a subject monitor) or implanted (such as a small patch over the neck/arm) and/or to a remote platform (such as a cloud platform). AEG devices may be coupled with acoustic, or sound conducting gels (or other materials) or can sense acoustic signals in air (airborne).

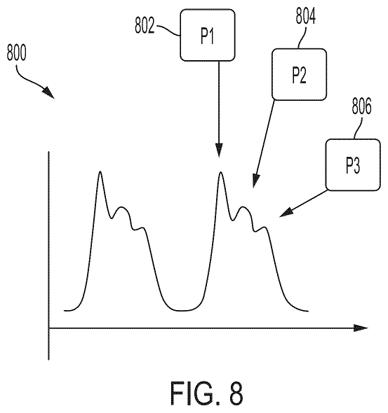

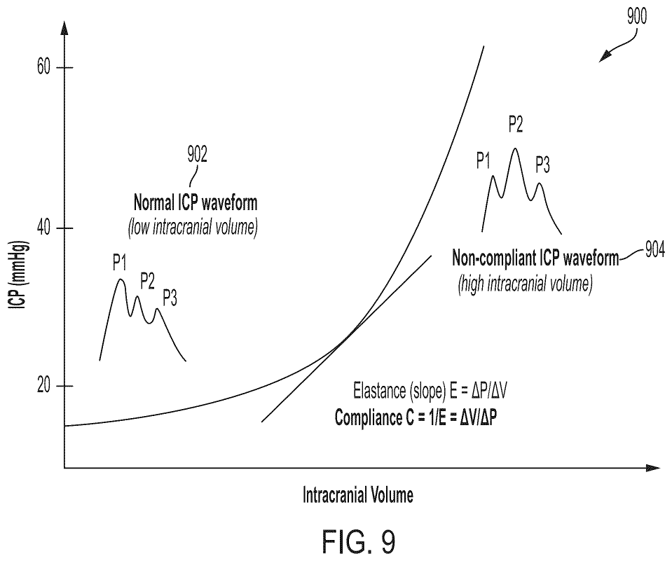

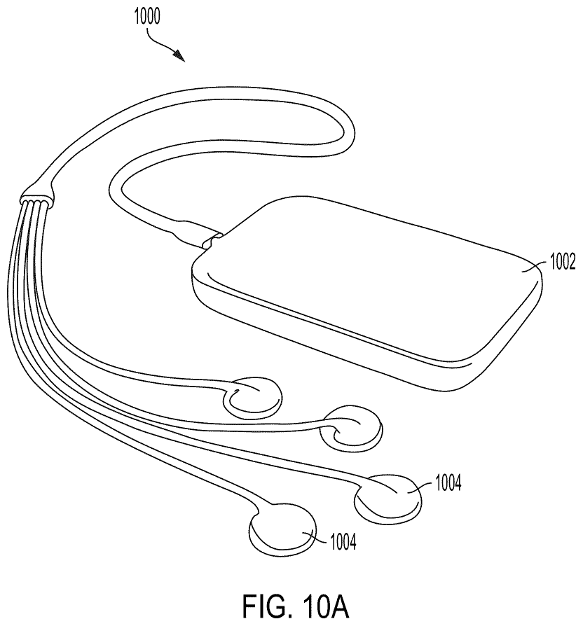

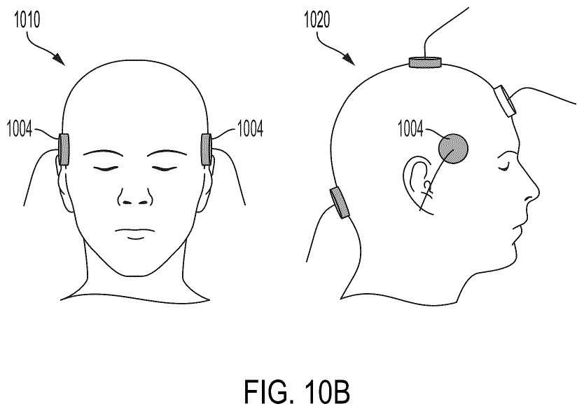

FIG. 10A shows an illustrative AEG device 1000 including a hub 1002 and multiple probes 1004 to access different brain compartments from various points over the head. In some embodiments, the probes 1004 are acoustic transducers. For example, a probe may be a piezoelectric transducer, capacitive micromachined ultrasonic transducer (CMUT), piezoelectric micromachined ultrasonic transducer (PMUT), electromagnetic acoustic transducer (EMAT), and/or another suitable acoustic transducer. The AEG device 1000 may be configured to measure acoustic signals in a subject's brain. The AEG device includes probes 1004 which may be configured to obtain acoustic measurement data by detecting acoustic signals in a subject's brain.

FIG. 10B shows illustrative arrangements of multiple AEG probes 1004 over the head of a subject. For example, in arrangement 1010, two probes 1004 are placed on the subject's head to access appropriate brain compartments. In another example, in arrangement 1020, fives probes 1004 are placed around the subject's head to get better access to different compartments of the brain of the person as compared to arrangement 1010. In some embodiments, the hub 1002 may include a processor. The processor may be configured to process signals obtained by the probes 1004. In some embodiments, the hub may include wireless communication circuitry (e.g., in a network interface card). The hub may be configured to use the wireless communication circuitry to communicate wirelessly with one or more other devices (e.g., of a cloud platform). example, the AEG device 1000 may be configured to continuously monitor of the subject's brain for more than 1 hour, 2, hours, 3, hours, 4, hours, 5 hours, six hours, 12 hours, 24 hours, a week, and/or any other suitable time period.

FIG. 10C shows a system 1030 in which the AEG device 1000 of FIG. 10A may be used, according to some embodiments of the technology described herein. The system 1030 includes the AEG device 1000, a cloud system 1032, a host system 1036, and a subject 1034. The AEG device 1000 may be in contact with the subject 1034. For example, the AEG device 1034 may be worn by the subject 1034 as described herein with reference to FIG. 10B. The AEG device 1034 may transmit information to the cloud system 1032 which may be accessed by clinicians (e.g., physicians, nurses, physicians' assistants, etc.) through a host system 1036.

In some embodiments, the subject 1034 may be a patient in a medical facility (e.g., a hospital, clinic, radiology center, etc.). For example, the subject 1034 may be a patient for a brain condition (e.g., brain trauma) and may need analysis of the brain 1034 for diagnosis or treatment. The AEG device 1000 may be used to monitor the brain of the subject 1034 (e.g., by monitoring ICP, elastance, or other parameters). In some embodiments, the AEG device 1000 may be configured to continuously monitor the brain of the subject 1034. For example, the AEG device 1000 may remain connected to the subject 1034 such that the AEG device 1000 is constantly monitoring the subject 1034. For example, the AEG device 1000 may monitor the subject 1034 for more than 1 hour, 2 hours, 3 hours, 4 hours, 5 hours, 6 hours, 12 hours, 1 day, 1 week, or other suitable time period.

The cloud system 1032 may be a set of one or more computing devices (e.g., server(s)) and storage devices (e.g., database(s)) in communication over a network (e.g., the Internet). For example, the cloud system 1032 may be a cloud based medical record system. The cloud system 1032 may be configured to receive information from the AEG device 1000. For example, the AEG device 1000 may stream data to the cloud system 1032. The cloud system 1032 may be configured to analyze data obtained from the AEG device 1000. The cloud system 1032 may be configured to provide information (e.g., obtained by analyzing data form the AEG device 1000) to one or more other devices (e.g., host system 1036). For example, the cloud system 1032 may provide information about the subject 1034 through an electronic medical record (EMR) user interface.

The host system 1036 may be a computing device configured to access the cloud system 1032. For example, the host system 1036 may be a laptop, desktop computer, smartphone, tablet, or other suitable computing device. The host system 1036 may include a network interface device that the host system 1036 uses to communicate with the cloud system 1032 through a network (e.g., the Internet). As an illustrative example, the host system 1036 may include a software application that may be used by users of the host system 1036 (e.g., clinicians) to access the cloud system 1032. In another example, the host system 1036 may receive information from the cloud system 1032.

FIG. 10D shows illustrative system architecture 1040 for an AEG device, according to some embodiments of the technology described herein. In the architecture 1040, subject 1042 (e.g., a patient) may have a network of probes 1044 (e.g., acoustics transducers) disposed on the patient's 1042 head. The network of probes 1044 may use transmit-receive electronics 1046 to transmit data acquired from monitoring the brain and/or skull of subject 1042. For example, the transmit-receive electronics 1046 may include wireless communication circuitry (e.g., radio, BLUETOOTH, infrared, or other type of wireless communication circuitry). In another example, the transmit-receive electronics 1046 may include an analog to digital converter. The transmit-receive electronics 1046 are connected to a processor 1048. The processor 1048 may be configured to process data received from the transmit-receive electronics 1046, and generate a display on a host application and/or a display 1050. For example, the process 1048 may generate a display of a waveform of one or more parameters (e.g., ICP).

It should be appreciated that the above-described AEG system is an exemplary system with which the smart-beam steering techniques described herein can be used. In particular, the smart-beam steering techniques, described herein including with respect to FIGS. 6-20, can be used in conjunction with any suitable system that passively or actively utilizes sound waves, as aspects of the technology described herein are not limited in this respect.

### Measurement Systems

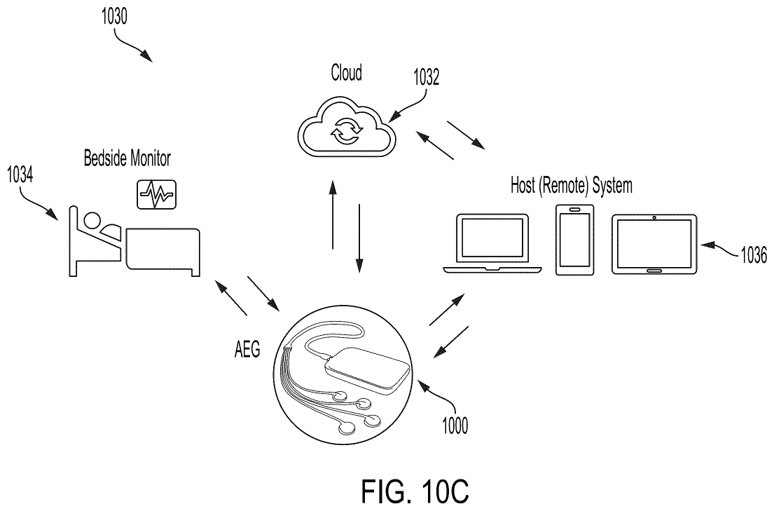

FIG. 11 shows an example of an ICP measurement system 1100, according to some embodiments of the technology described herein. The ICP measurement system 1100 includes probes 1004, a CBF measurement module 1100A, an input generation module 1100B, and an ML model 1100C.

As shown in FIG. 11, the ICP measurement system 1100 obtains, as input, acoustic measurement data 1102, an ABP measurement 1104, and context information 1106, and determines an ICP measurement 1108. In some embodiments, the ICP measurement system 1100 may obtain, as input, other inputs instead of or in addition to ABP measurement 1104, acoustic measurement data 1102, and context information 1106. In some embodiments, the ICP measurement system 1100 may not obtain context information 1106 as input.

The ABP measurement 1104 may be obtained using one of various techniques. In some embodiments, the ABP measurement 1104 may be obtained using an invasive technique. For example, the ABP measurement 1104 may be obtained using an arterial catheterization device or other invasive technique. In some embodiments, the ABP measurement 1104 may be obtained using a non-invasive technique. For example, the ABP measurement 1104 may be obtained using arterial applanation tonometry, vascular unloading technology, or other non-invasive technique. In some embodiments, the ABP measurement 1104 may be obtained (e.g., received) from outside of the ICP measurement system 1100. In some embodiments, the ABP measurement 1104 may be determined by the ICP measurement system 1100.

In some embodiments, the ABP measurement 1104 may be a set of multiple ABP values obtained over a period of time. For example, the ABP may be monitored continuously over a period of time, and the ICP measurement system 110 may receive the measured ABP values over the period of time. In some embodiments, the ICP measurement system 1100 may be configured to obtain the ABP measurement 1104 in real time. For example, the ABP measurement 1104 may be a continuous transmission of ABP values transmitted to the ICP measurement system 100 as they are measured by a device.

The acoustic measurement data 1102 may include data obtained by guiding an acoustic beam towards one or more regions of a subject's brain, and detecting one or more acoustic signals from the region(s) of the subject's brain. In some embodiments, the acoustic measurement data 1102 may be obtained using “smart beam-steering” (SBS) which: (1) applies an acoustic signal (e.g., an ultrasound signal) to a region of the brain; and (2) uses an acoustic transducer (e.g., a piezoelectric transducer, a capacitive micromachined ultrasonic transducer, a piezoelectric micromachined ultrasonic transducer, and/or other suitable transducer) to detect an acoustic signal from a region by forming a beam in a direction. For example, the acoustic measurement data 1102 may be obtained by using transcranial doppler (TCD) ultrasound to transmit an acoustic signal to a region of a subject's brain, and using a transducer to detect a responsive acoustic signal. SBS devices and techniques are described in.

The context information 1106 may include various types of information. For example, the context information 1106 may include a mean ABP, a vessel diameter, a subject's age, a subject's gender, allergies, medication history, dimensions and geometry of the subject's head, height, weight, indication of the subject's previous medical condition(s), surgical history, family history, immunization history, developmental history, and/or other information. As indicated by the dotted line of the context information 1106 in FIG. 11, in some embodiments, the ICP measurement system 1100 may not obtain context information 1106. In these embodiments, the ICP measurement system 1100 may determine the ICP measurement 1108 without using context information 1106.

The ICP measurement system 1100 may include any suitable computing device to implement its modules. For example, the ICP measurement system 1100 may include a desktop computer, a laptop, smartphone, tablet, or other suitable computing device. In some embodiments, the ICP measurement system 1100 may be embedded in an AEG device (e.g., AEG device 1000 described herein with reference to FIGS. 10A-D). For example, the AEG device may be implemented on an embedded circuit within the AEG device. In another example, the AEG device may be implemented using a processor and memory of the AEG device. In some embodiments, the ICP measurement system 1100 may be a cloud based system (e.g., cloud system 1032 described herein with reference to FIG. 10C).

The ICP measurement system 1100 may include memory storing parameters of the machine learning model 1100C. In some embodiments, the parameters may be learned parameters obtaining by applying a training algorithm to a set of training data. Example training techniques are described herein.

The CBF measurement module 1100A may be configured to use the acoustic measurement data 1102 to determine a CBF measurement of a subject's brain. The CBF measurement provides a measurement of volumetric blood flow in a portion of the subject's brain. In the example of FIG. 11, the CBF measurement module 1100A determines a CBFV measurement 1100A-1 of the subject's brain indicating the volumetric blood flow in the portion of the subject's brain. The ICP measurement system 1100 may be configured to process data obtained from detecting acoustic signals (e.g., from probes 1004 of device 1000 described herein with reference to FIG. 10) to determine the CBF measurement. The ICP measurement system 1100 may be configured to analyze the acoustic measurement data 1102 to determine the CBFV measurement 1100A-1.

In some embodiments, the CBFV measurement 1100A-1 may include a signal wave. For example, the CBFV measurement 1100A-1 may include a CBFV value measured at various points in a period of time. In some embodiments, the CBFV measurement 1100A-1 may include a mean CBFV measurement. For example, the CBFV measurement 1100C-1 may be a mean CBFV measurement in a period of time.

In some embodiments, the CBF measurement module 1100A may be configured identify an envelope of a CBF measurement (e.g., a CBFV signal). The envelope values may be used by the input generation module 1100B for generation of input. For example, the input generation module 1100B may use envelope CBFV values as input to the ML model 1100C.

In some embodiments, the CBF measurement module 1100 may be configured to identify points associated with heartbeats of the subject in a CBF measurement. The identified heartbeat points may be used by the input generation module 1100B for generation of input for the ML model 1100C. For example, the input generation module 1100B may use points in a CBFV signal associated with heartbeats to segment CBF measurement data into multiple inputs.

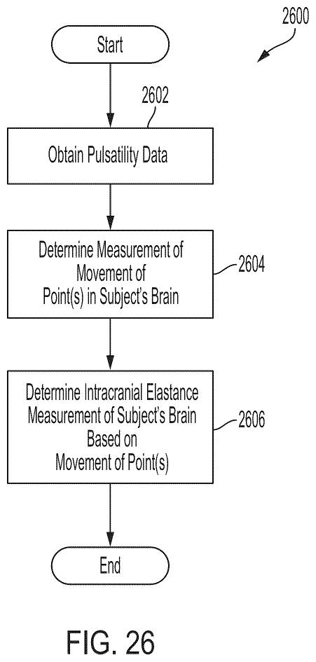

FIG. 27 shows an example machine learning model 2700 for identifying an envelope of a CBF measurement, according to some embodiments of the technology described herein. In the example of FIG. 27, the CBF measurement is a CBFV signal 2702. The machine learning model 2700 is an autoencoder including an encoder 2704 and a decoder 2706. The CBFV signal 2702 may be provided as input to the encoder 2704 which generates an encoded output. The encoded output may then be provided as input to a decoder 2706 to obtain output indicating a segmented envelope of the CBFV signal 2702 and/or points associated with heart beats in the CBFV signal 2702. For example, the machine learning model 2700 may output, for each of a series of CBFV values in the CBFV signal 2702, an indication of whether the CBFV value is on the envelope and/or whether the CBFV valuc and/or an indication of whether the CBFV value is associated with a heartbeat of the subject.

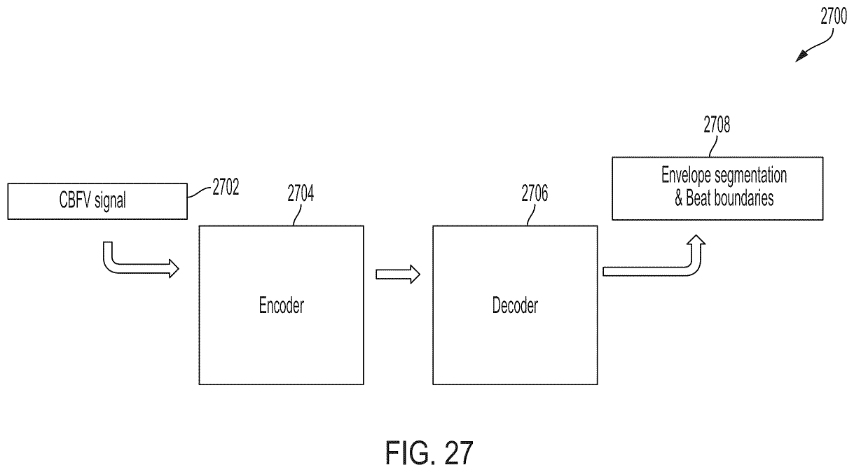

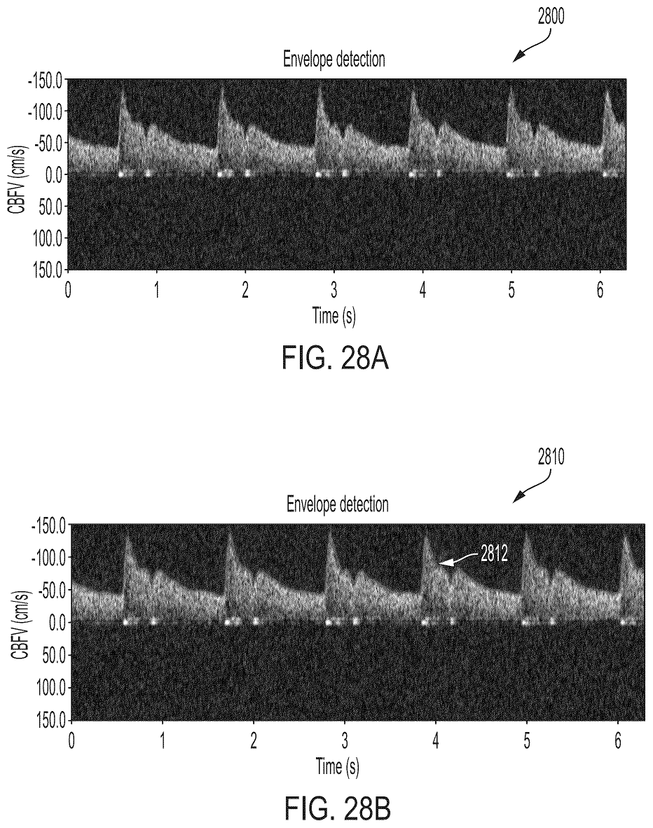

FIG. 28A shows a plot 2700 of an example CBFV signal that may be provided as input to the machine learning model 2700 of FIG. 27. The CBFV signal may be determined through acoustic measurement data (e.g., obtained using SBS techniques described herein). FIG. 28B shows a plot 2810 showing the CBFV signal of FIG. 28A with the segmented envelope 2812 of the CBFV signal marked. The envelope 2812 may have been segmented using the machine learning model 2700 of FIG. 27.

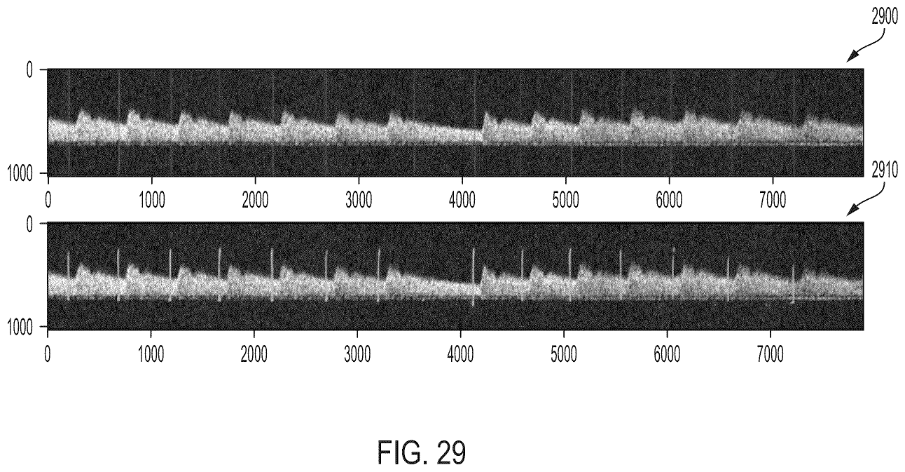

FIG. 29 shows a plot 2900 of a CBFV signal with lines marking points that are associated with heartbeats. FIG. 29 further shows a plot 2910 indicating points in the CBFV signal of FIG. 29 identified by the machine learning model 2700 as being associated with heartbeats. The identified heartbeat points may be used to segment the CBFV signal into portions (e.g., that can be used as inputs for the ML model 1100C).

In some embodiments, the input generation module 1100B may be configured to use the ABP measurement 1104, a CBF measurement (e.g., CBFV measurement 1100C-1) determined by the CBF measurement module 1100A, and/or the context information 1106 to generate input 1100B-1 to the ML model 1100C. For example, the input generation module 1100B may pre-process measurement data (ABP measurement data and/or CBF measurement data) to generate the input 1100B-1. In some embodiments, the input generation module 1100B may be configured to generate the input 1100B-1 by determining one or more parameters from the ABP measurement data and/or the CBF measurement. For example, the input generation module 1100B may determine a mean CBFV valuc and/or a mean ABP value to include in the input 1100B-1. As another example, the input generation module 1100B may determine a variance, median, and/or other suitable parameter using ABP measurement data and/or CBF measurement data to include in the input 1100B-1.

In some embodiments, the input generation module 1100B may be configured to transform measurement data into a frequency domain. The input generation module 1100B may use values from the frequency domain to determine ICP. For example, the input generation module 1100B may use the frequency domain signal(s) to determine a mean ABP and/or a mean CBFV. In some embodiments, the input generation module 1100B may be configured to determine parameter values in a time domain of ABP and/or CBFV measurement data. For example, the system may determine parameters such as rise-time, peak-to-peak time, derivative, and/or other parameters of a time domain signal.

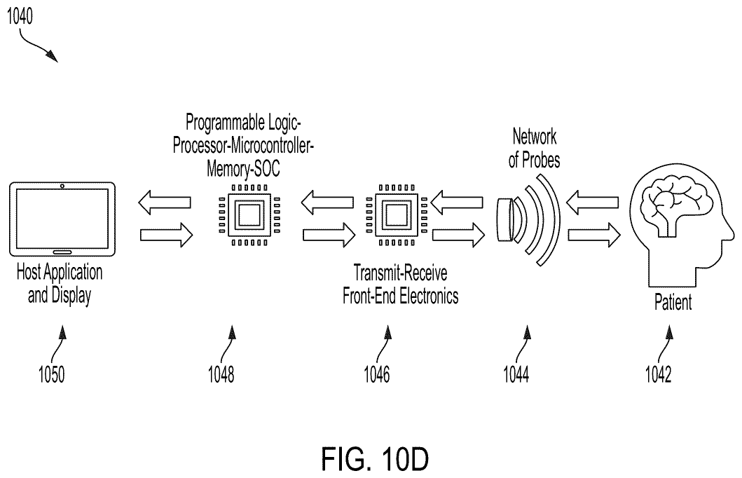

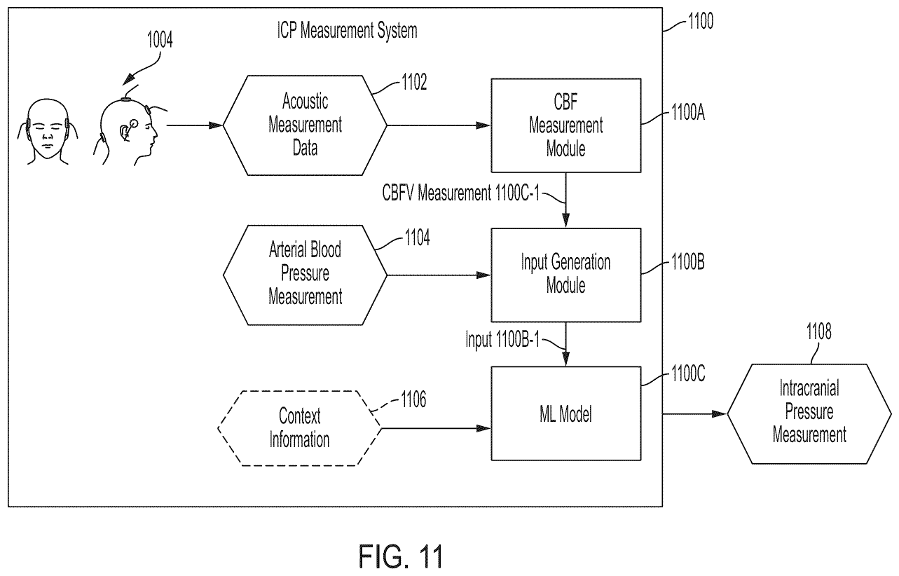

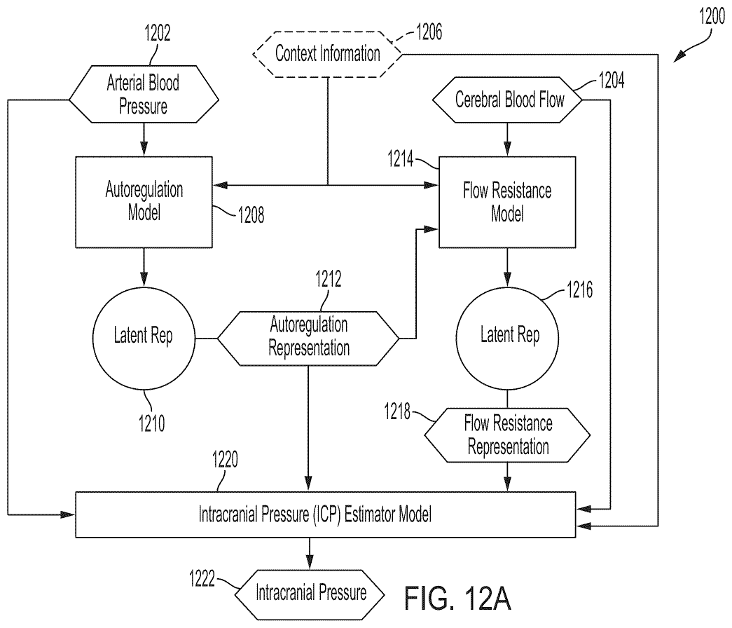

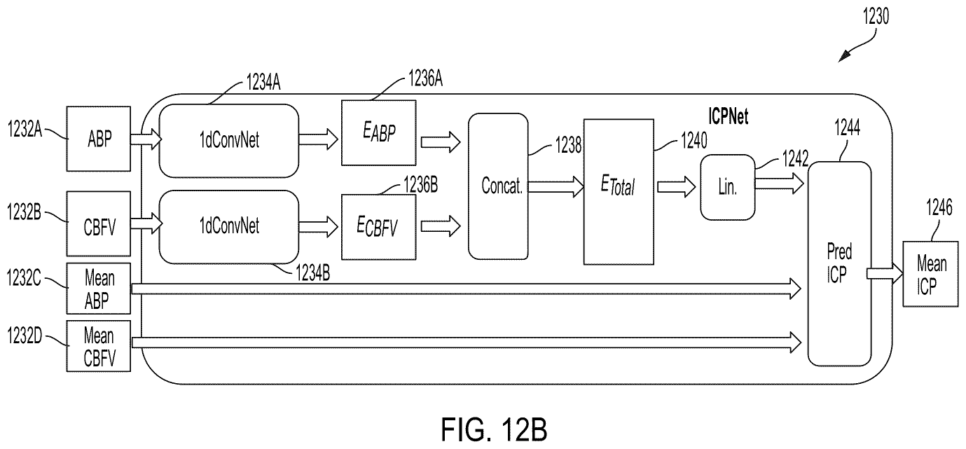

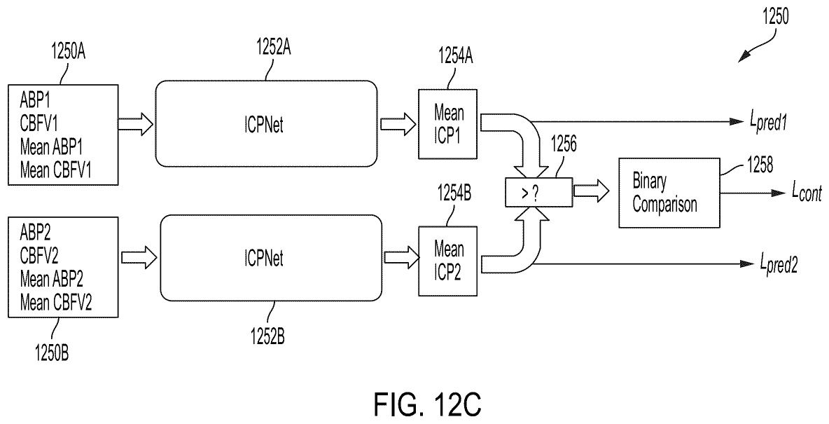

The input generation module 1100B may be configured to provide the input 1100B-1 to the ML model 1100C to obtain an output of the ML model 1100C indicative of the ICP measurement 1108. In some embodiments, the ML model 1100C may be a physics guided ML model that is based on a model of the brain. As an illustrative example, the physics guided ML model may be based on a hemodynamic model of the brain (e.g., hemodynamic model 500 described herein with reference to FIG. 5). The structure of the physics guided ML model may be based on the structure of the model. The physics guided ML model may include multiple different ML models representing respective aspects of a brain. For example, if the hemodynamic model includes a resistor, capacitor, and inductor representing various aspects of a brain, the physics guided ML model may include a respective ML model for each of the components. In some embodiments, the physics guided machine learning model of the ICP measurement system 1100 may be based on any suitable model of the brain. Example ML models that may be used by the ICP measurement system 1100 are described herein with reference to FIGS. 12A-12E.

In some embodiments, the ML model 1100C may be based on an RC and/or an RLC circuit model of the brain. Equations corresponding to the model may be used to determine an ICP measurement using ABP and CBFV values. The system 1100 may solve for circuit parameters (e.g., resistance, capacitance, and/or inductance) using time domain and/or frequency domain ABP and CBFV values. As an illustrative example, the system 1100 may solve circuit parameters in the frequency domain at principal peak frequencies using a least-squares solution. The least-squares solution may use mean-subtracted ICP, ABP, and CBFV. The mean ICP may be estimated using a resistance value in conjunction with mean ABP and mean CBFV. As another example, the system 1100 may determine mean ICP by estimating circuit parameters by extracting time-domain features of ABP and CBFV signals (e.g., rise-time, peak-to-peak time, derivatives of signals, and/or other suitable features).

In some embodiments, the ML model 1100C may include a decision tree model. In some embodiments, the decision tree model may receive, as input, features extracted from a frequency domain (e.g., Fourier transform) of ABP and/or CBFV signals. For example, the features may be real and imaginary parts of a frequency domain signal at peak frequencies. In some embodiments, the ML model 1100C may receive features extracted from a time domain of the ABP and/or CBFV signals. In some embodiments, the ML model 1100C may include a support vector machine (SVM), a neural network, a decision tree model, a Naïve Bayes model, or other suitable ML model. In some embodiments, the ML model 1100C may be a combination of one or more models.

The ICP measurement 1108 may be one or more ICP values determined by the ICP measurement system 1100. In some embodiments, the ICP valuc(s) may be ICP values over a period of time. The ICP value(s) may be a time series of ICP values. For example, the ICP measurement 1108 may be a waveform of ICP values over the period of time. In some embodiments, the ICP measurement system 1100 may be configured to output the ICP measurement 1108 periodically. For example, the ICP measurement system 1100 may output an ICP measurement every 1 second, 10 seconds, 30 seconds, 1 minute, 2 minutes, 5 minutes, 10 minutes, or other frequency. In some embodiments, the ICP measurement system 1100 may be configured to output the ICP measurement 1108 in response to receiving one or more inputs (e.g., ABP measurement 1104 input, acoustic measurement data 1102 input, and/or context information 1106 input). For example, the ICP measurement 110 may update the ICP measurement 1108 in response to new a new input.

FIG. 12A shows an example physics guided ML model 1200 that may be used to obtain an ICP measurement. For example, the ML model 1200 may be used by the ICP measurement system 1100 of FIG. 11 (e.g., as ML model 1100C), according to some embodiments of the technology described herein. The physics guided ML model 1200 may be based on a resister capacitor (RC) circuit model of the brain (e.g., model 500 described herein with reference to FIG. 5).

As shown in FIG. 12A, the physics guided ML model 1200 includes multiple models including: an auto regulatory mechanism model 1208 (represented by the capacitor of the RC circuit), a flow resistance model 1214 (represented by the resistor of the RC circuit), and an ICP estimator model 1222 which generates an output indicating an ICP measurement 1222 using outputs of the auto regulatory mechanism model 1208 and the flow resistance model 1214. In some embodiments, each of the ML models 1208, 1214, 1222 may be a neural network. For example, the ML model may be a deep neural network (DNN), a convolutional neural network (CNN), a recurrent neural network (RNN), and/or another suitable neural network. In some embodiments, each of the autoregulation model 1208 and the flow resistance model 1214 may be an encoder of a respective autoencoder. In some embodiments, the encoder may be a variational encoder of a respective variational autoencoder. The variational autoencoder may be trained to generate an output indicating a probability distribution.

The autoregulation model 1208 may be trained to represent the cerebral autoregulation of a brain. For example, the autoregulation model 1208 may represent a capacitor in an RC circuit model of the brain. As shown in FIG. 12A, the autoregulation model 1208 may receive as input an ABP measurement 1202 and context information 1206. The autoregulation model 1208 may be configured to generate a latent representation 1210 using the ABP measurement 1202 and the context information 1206. For example, the ABP measurement 1202 may be a set of one or more ABP values (e.g., an ABP waveform) obtained in a period of time (e.g., provided in a vector). In some embodiments, the autoregulation model 1208 may be configured to generate the latent representation 1210 without using context information 1206 as indicated by the dotted lines of the context information 1206. In some embodiments, the latent representation 1210 may be used to obtain an autoregulation representation 1212 of the brain. The latent representation 1210 may include values encoding operation of the cerebral autoregulation. In some embodiments, the ICP measurement system 1100 may be configured to use the latent representation 1210 as the autoregulation representation 1212. In some embodiments, the latent representation 1210 may be an indication of a probability distribution. For example, the latent representation 1210 may be a mean and variance vector indicating a Gaussian probability distribution representing the cerebral autoregulation of the brain. The ICP measurement system 1100 may be configured to sample the probability distribution to obtain the autoregulation representation 1212. For example, the ICP measurement system 1100 may obtain one or more samples from the probability representation as the autoregulation representation 1212.

The flow resistance model 1214 may be trained to represent the flow resistance of a brain. For example, the flow resistance model 1214 may represent a resistor in an RC circuit model of the brain. As shown in FIG. 12A, the flow resistance model 1214 may receive as input a CBF measurement 1204 (e.g., determined using acoustic measurement data 1102), context information 1206, and an autoregulation representation 1212. For example, the flow resistance model 1214 may receive a vector including the autoregulation representation 1212, the cerebral blow measurement 1204, and the context information 1206. The flow resistance model 1214 may be configured to generate a latent representation 1216. The latent representation 1216 may include values encoding flow resistance of the brain. In some embodiments, the ICP measurement system 1100 may be configured to use the latent representation 1216 as the flow resistance representation 1218. In some embodiments, the latent representation 1216 may be an indication of a probability distribution. For example, the latent representation 1216 may be a mean and variance vector indicating a Gaussian probability distribution representing flow resistance of the brain. The ICP measurement system 110 may be configured to sample the probability distribution to obtain the flow resistance representation 1218. For example, the ICP measurement system 1100 may obtain one or more samples from the probability distribution to obtain the flow resistance representation 1218.

The ICP estimator model 1220 may be trained to determine an ICP measurement 1222. As shown in FIG. 12A, the ICP estimator model 1220 may receive as input the autoregulation representation 1212 and the flow resistance representation 1218. For example, the ICP estimator model 1220 may receive a vector including the autoregulation representation 1212 and the flow resistance representation 1218. The ICP estimator model 1220 may be trained to use the autoregulation representation 1212 and the flow resistance representation 1218 to generate the ICP measurement 1222. In some embodiments, the ICP estimator model 1220 may be trained to output an ICP distribution. The ICP measurement system 1100 may be configured to determine the ICP measurement 1222 using the ICP distribution. For example, the ICP measurement system 1100 may obtain a mean, maximum, or median of the ICP distribution to obtain the ICP measurement 1222.

In some embodiments, the physics guided ML model 1200 may be trained using a supervised learning technique. The physics guided ML model 1200 may be trained by: (1) obtaining training data including ABP measurements, CBF measurements, context information, and corresponding ICP measurements; and (2) training the model 1200 by applying a supervised learning technique to the training data. For example, the physics guided ML model 1200 may be trained using stochastic gradient descent in which the parameters of the model are iteratively updated to minimize a loss function (e.g., mean squared error, L2 loss, quadratic loss, and/or mean absolute error). In each iteration, a training system may: (1) use a sample ABP measurement, a CBF measurement, and a context information from the training data to obtain an output ICP measurement; and (2) update parameters of the model based on a difference between the output ICP measurement and the ICP measurement corresponding to the sample. The training system may iterate until a maximum number of iterations are performed and/or until a threshold value of the loss function is achieved. In some embodiments, the physics guide ML model 1200 may be trained by applying a semi-supervised learning technique to the training data.

FIG. 12B shows another example ML model 1230 that may be used by the ICP measurement 1100 described herein with reference to FIG. 11 (e.g., as ML model 1100C).

As shown in FIG. 12B, the ML model 1230 receives, as input, a set of ABP measurements 1232A, a set of CBFV measurements 1232B, a mean ABP 1232C, and a mean CBFV 1232D. The set of ABP measurements 1232A and set of CBFV measurements 1232B may be obtained as described herein with reference to FIG. 11. In some embodiments, the ABP measurements 1232A may be a set of ABP values over a period of time (e.g., a time series of ABP values). The mean ABP 1232C may be a mean of the set of ABP measurements. In some embodiments, the set of CBFV measurements 1232B may be a set of CBFV values over a period of time (e.g., a time series of CBFV values). The mean CBFV 1232D may be a mean of the set of CBFV measurements 1232D.

As shown in FIG. 12B, the ML model 1230 includes a first convolutional network 1236A and a second convolutional network 1234B. In the example of FIG. 12B, each of the convolutional networks is a one dimensional. In some embodiments, the convolutional networks 1234A, 1234B may be of another suitable dimension. The first convolutional network 1234A receives the set of ABP measurements 1232A as input and the second convolutional network 1234B receives the set of CBFV measurements 1232B as input. The convolutional networks 1234A, 1234B generate respective outputs 1236A, 1236B. The output 1236A may be a representation of the set of ABP measurements 1232A and the output 1236B may be a representation of the set of CBFV measurements 1232B. The ML model then combines the outputs 1236A, 1236B of the convolutional networks 1234A, 1234B. In the example of FIG. 12B, the ML model 1230 concatenates 1238 the outputs 1236A, 1236B to obtain a combined representation 1240 of the outputs. The combine representation 1240 is provided as input to a neural network 1242. For example, the neural network 1242 may be a feed-forward neural network. The output of the neural network 1242 may be input, along with the mean ABP valuc 1232C and the mean CBFV value 1232D to an ICP predictor 1244 to obtain an output mean ICP measurement 1246.

In some embodiments, the ICP predictor 1244 may be an ML model. For example, the ICP predictor 1244 may be a decision tree model. As another example, the ICP predictor 1244 may be a support vector machine (SVM), a neural network, Naïve Bayes model, or other suitable ML model. In some embodiments, the ICP predictor 1244 may be a circuit based model (e.g., an RLC or RC model) in which the ICP measurement can be determined by solving equations for circuit parameters, and using ABP and CBFV measurements in conjunction with the circuit parameters to determine the ICP measurement 1246.

FIG. 12C shows another example ML model 1230 that may be used to obtain an ICP measurement (e.g., by ICP measurement system 1100 described herein with reference to FIG. 11).

The ML model 1250 is a contrastive convolutional network that uses the ML model 1230 of FIG. 12B (also referred to herein as an “ICP network”) as a building block. The ML model 1250 includes a first ICP network 1252A and a second ICP network 1252B.

Each of the ICP networks 1252A, 1252B receives a respective set of inputs 1250A, 1250B. Each of the set of inputs 1250A, 1250B includes a set of ABP measurements, a set of CBFV measurements, a mean ABP, and a mean CBFV. In some embodiments, the set of inputs 1250A, 1250B may be associated with a respective period of time. For example, the set of inputs 1250A may include inputs for a first period of time and the set of inputs 1250B may include inputs for a second period of time different from the first period of time. The inputs 1250A, 1250B are provided to the ICP networks 1252A, 1252B to determine the mean ICP measurements 1254A. 1254B. The model 1250 then performs a comparison 1256 to generate a comparison result 1258. In the example of FIG. 12C, the result 1258 is a binary comparison that may be obtained by determining whether the mean ICP measurement 1254A is greater than the mean ICP measurement 1254B. As another example, the comparison 1256 may be a difference between the mean ICP measurements 1254A, 1254B, a function of the difference (e.g., a cosine, normalization), or other suitable comparison.

In some embodiments, the weights of the ICP networks 1252A, 1252B may be tied such that the comparison result 1258 may be used to update weights of the ICP networks 1254A, 1254B during training. This may allow each of the ICP networks 1254A, 1254B to improve their capability to predict a mean ICP (e.g., by learning common features of various parameters).

FIG. 12D shows another example ML model 1260 that may be used to obtain an ICP measurement (e.g., by ICP measurement system 1100 described herein with reference to FIG. 11). The ML model 1260 is a neural network comprising of: (1) a convolutional neural network 1262; (2) a long short-term memory (LSTM) network or a transformer network; and (2) a convolutional network 1266.

In some embodiments, the model 1260 may receive, as input, a set of ABP measurements 1260A and a set of CBFV measurements 1260B. For example, each of the set of measurements 1260A, 1260B may be measurements in a period of time (e.g., a time series of values). In some embodiments, the inputs 1260A, 1260B may be parameters determined from respective sets of ABP and CBFV measurements. For example, the inputs 1260A, 1260B may be frequency domain values obtained by transforming time domain measurement values (e.g., by input generation module 1100B). In some embodiments, the amount of content in the frequency domain may be reduced. For example, ⅙ of content of a frequency domain transformation of a set of measurements may be used as input.

The inputs 1260A, 1260B are provided to the CNN 1262A. The output of the CNN 1262A is then provided to the LSTM/transformer 1264. The output of the LSTM/Transformer 1264 is then provided to the CNN 1266 to obtain an ICP measurement 1268. In the example of FIG. 12D, the ICP measurement 1268 is a set of ICP measurements over a period of time (e.g., a time series of values). The set of ICP measurements over a period of time may also be referred to as a “full-wave ICP”.

FIG. 12E shows another example ML model 1270 that may be used to obtain an ICP measurement (e.g., by ICP measurement system 1100 described herein with reference to FIG. 11). The ML model 1270 includes a Siamese neural network 1274 that includes a convolutional neural network 1274A and a LSTM 1274B, and two fully connected layers 1278A, 1278B. The neural network 1270 of FIG. 12E determines a representation of an ICP measurement (“ICP embedding”) and a representation of a subject (“subject embedding”). The Siamese neural network 1274 may be trained using the neural network 1270 and then used to determine a measurement of ICP (e.g., in the ML model 1260 of FIG. 12D).

The neural network 1270 is trained to distinguish between mean ICP level and subject. The neural network 1270 generates a respective embeddings for the ICP level and the subject. As shown in FIG. 12E, the network 1274 receives as input: (1) an ABP measurement pair 1274A consisting of a set of ABP measurements from one subject (“ABP1”) and a set of ABP measurements from another subject (“ABP2”); and (2) a CBFV measurement pair 1274B consisting of a set of CBFV measurements from one subject (“CBFV1”) and a set of CBFV measurements from another subject (“CBFV2”). The neural network 1274 outputs a pair of embeddings for each of the subjects. For each subject, one embedding represents a mean ICP measurement of the subject and a second embedding represents the subject.

The neural network 1270 includes two fully connected layers 1278A, 1278B. The fully connected layer (FC) 1278A receives as input the ICP embeddings associated with each subject and outputs an indication of whether the two subjects have the same ICP levels. The fully connected layer 1278B receives as input the subject embeddings associated with each subject and outputs an indication of whether two subjects are the same. In some embodiments, the neural network 1270 may be implemented as a variational model that outputs a probability distribution for each embedding. For example, the variational model may output a probability distribution representing mean ICP level of a subject and a probability distribution representing the subject.

In some embodiments, ML models described herein may be trained using a supervised learning technique. An ML model may be trained by: (1) obtaining training data including sample inputs (e.g., ABP and CBF measurement data) and corresponding outputs (e.g., ICP measurements); and (2) applying a supervised learning technique to the training data. For example, the ML model may be trained using stochastic gradient descent in which the parameters of the model are iteratively updated to minimize a loss function (e.g., mean squared error, L2 loss, quadratic loss, and/or mean absolute error). In each iteration, a training system may: (1) use an input sample from the training data to obtain an output measurement; and (2) update parameters of the model based on a difference between the output measurement and the measurement corresponding to the input sample from the training data. The training system may iterate until a maximum number of iterations are performed and/or until a threshold value of the loss function is achieved. In some embodiments, the ML model may be trained by applying a semi-supervised learning technique to the training data. In some embodiments, the ML model may be trained by applying an unsupervised learning technique to the training data (e.g., a clustering technique).

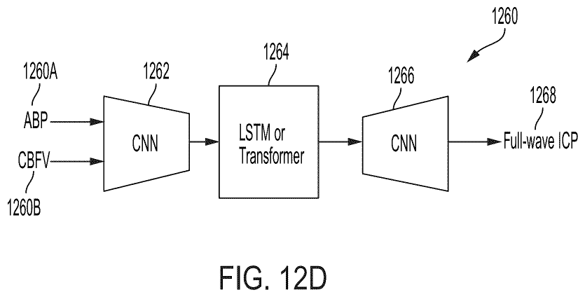

FIG. 13 shows an example process 1300 to determine an ICP measurement (e.g., mean ICP and/or full-wave ICP), according to some embodiments of the technology described herein. In some embodiments, process 1300 may be performed by the ICP measurement system 1100 of FIG. 11.

At block 1302, the system obtains an ABP measurement (e.g., ABP measurement 1104 described herein with reference to FIG. 11). In some embodiments, the system may be configured to obtain the ABP measurement by receiving the ABP measurement from a measurement device. For example, the system may receive, through a connection with the measurement device, data including the ABP measurement. In some embodiments, the system may be configured to receive an ABP measurement periodically (e.g., every 1 second, 10 seconds, 30 seconds, 1 minute, 2 minutes, 5 minutes, 10 minutes, or other suitable frequency). For example, the system may continuously receive ABP measurements from a measurement device. In some embodiments, the system may determine an ABP measurement by using acoustic measurement data. Examples of determining an ABP measurement using acoustic measurement data is described herein.

At block 1304, the system obtains acoustic measurement data (e.g., acoustic measurement data 1102). In some embodiments, the system may be configured to obtain acoustic measurement data obtained by an AEG device (e.g., AEG device 1000 described herein with reference to FIGS. 10A-D.). For example, the system may obtain acoustic measurement data obtained through probes of the AEG device. In some embodiments, the system may be configured to obtain the acoustic measurement data as it is being captured by the AEG device. For example, the system may obtain the acoustic measurement data being captured from a brain of a subject who is being monitored using the AEG device. In some embodiments, the system may be configured to receive the acoustic measurement data periodically (e.g., every 1 second, 10 seconds, 30 seconds, 1 minute, 2 minutes, 5 minutes, 10 minutes, or other suitable frequency). For example, the system may continuously receive acoustic measurement data from the AEG device.

At block 1308, the system obtains context information (e.g., context information 1106). In some embodiments, the system may be configured to obtain the context information from another device. For example, the system may obtain context information (e.g., subject weight, age, height, surgical history, medication history, and/or other context information) from an electronic medical record (EMR) system. In some embodiments, the system may be configured to access the context information from memory of the system. In some embodiments, the system may be configured to obtain the context information by analyzing acoustic measurement data and/or ABP measurement data. For example, the system may determine a mean or median ABP measurement by analyzing previously received ABP measurement data. As indicated by the dotted lines of block 1308, in some embodiments, the system may not obtain context information. The process 1300 may procced from block 1306 to block 1310 without performing the acts of block 1308.

It should be appreciated that the ABP measurement, acoustic measurement data and context information may be obtained in any order and/or in parallel. In some embodiments, the system may obtain the ABP measurement, acoustic measurement, and context data in any order.

At block 1306, the system uses the acoustic measurement data to determine a CBF measurement of the subject's brain. In some embodiments, the CBF measurement may be a measurement of CBFV (e.g., a CBFV signal wave). The system may be configured to determine the CBF measurement by analyzing the acoustic measurement data. For example, the system may analyze detected acoustic signals to determine CBF values over a period of time. The system may determine a CBF waveform based on the acoustic measurement data. For example, the system may determine the CBF waveform based on the acoustic measurement data using estimation of doppler shift through quadrature demodulation, side-band filtering, heterodyne demodulation, phase-estimation techniques, auto-correlation techniques, cross-correlation techniques, and/or other techniques.

In some embodiments, the system may be configured to identify an envelope of a CBF measurement signal (e.g., a CBFV measurement signal). For example, the system may use a machine learning model (e.g., as described herein with reference to FIG. 27) trained to segment the envelope from the CBF measurement signal using the acoustic measurement data. In some embodiments, the system may be configured to segment a CBF measurement signal by heart beats. For example, the system may use a machine learning model (e.g., as described herein with reference to FIG. 27) to identify heart beats in a timeline of the CBF measurement signal.

At block 1310, the system uses the CBFV measurement and the ABP measurement to generate input to a ML model. For example, the system may determine one or more parameters (e.g., mean CBFV, mean ABP, max CBFV, max ABP, min CBFV, min ABP, and/or other parameters) using the CBFV and ABP measurements. The system may use the determined parameter(s) as input to the ML model. In some embodiments, the system may be configured to use a CBFV measurement and/or ABP measurement (e.g., time series values) or portions thereof as input.

At block 1312, the system provides the input to the ML model to obtain an output indicating an ICP measurement (e.g., mean ICP and/or a full-wave ICP signal). The system may be configured to output the ICP measurement (e.g., to another device for display).

As an illustrative example, if the system uses the physics guided ML model 1200 described herein with reference to FIG. 12A, system may be configured to: (1) generate input for the autoregulation model 1208 using the ABP measurement and context information; (2) provide the input to the autoregulation model 1208 to obtain an autoregulation representation 1212 (e.g., using latent representation 1210); (3) generate input for the flow resistance model 1214 using the autoregulation representation 1212, context information 1206, and the cerebral blood flow 1204; (4) provide the input to the flow resistance model 1214 to obtain a flow resistance representation 1218 (e.g., using latent representation 1216); and (5) provide the autoregulation representation and flow resistance representation as input to the ICP estimator mode 1220 to obtain the ICP measurement 1222.

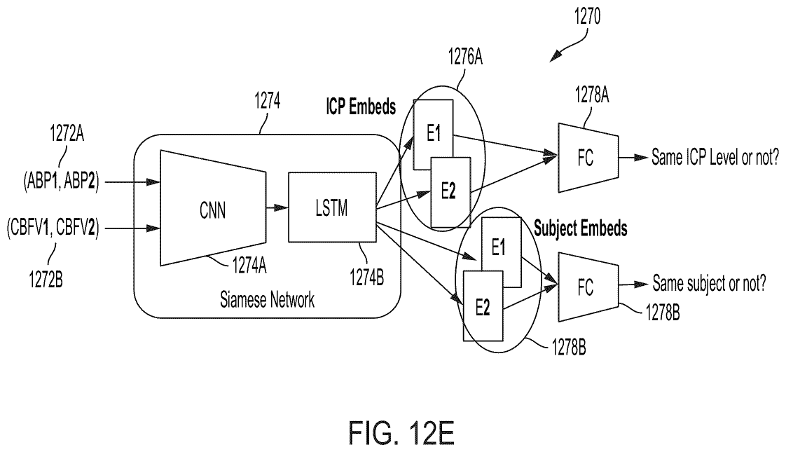

FIG. 14 shows an example joint ICP/ABP measurement system 1400, according to some embodiments of the technology described herein. The ICP/ABP measurement system 1400 includes acoustic measurement probes 1004 (e.g., of an AEG device), an ABP measurement module 1400A, a CBF measurement module 1400B, an input generation module 1400C, and an ML model 1400D. As shown in FIG. 14, the ICP/ABP measurement system 1100 obtains, as input, acoustic measurement data 1402 and context information 1404, and determines an ICP measurement 1406 and an ABP measurement 1408 of a subject.

The acoustic measurement data 1402 may be acoustic measurement data 1102 described herein with reference to FIG. 11. The acoustic measurement data 1102 may be obtained using probes 1004 described herein with reference to FIG. 10 (e.g., by using SBS techniques described herein). The context information 1404 may be context information 1106 described herein with reference to FIG. 11. The ICP measurement 1406 may be as described with reference to the ICP measurement 1108 of FIG. 11.

The ICP/ABP measurement system 1400 may include suitable computing device to implement its modules. For example, the ICP/ABP measurement system 1400 may be a desktop computer, a laptop, smartphone, tablet, or other suitable computing device. In some embodiments, the ICP/ABP measurement system 1400 may be embedded in an AEG device (e.g., AEG device 1000 described herein with reference to FIGS. 10A-D). For example, the AEG device may be implemented on an embedded circuit within the AEG device. In another example, the AEG device may be implemented using a processor and memory of the AEG device. In some embodiments, the ICP/ABP measurement system 1400 may be a cloud based system (e.g., cloud system 1032 described herein with reference to FIG. 10C).

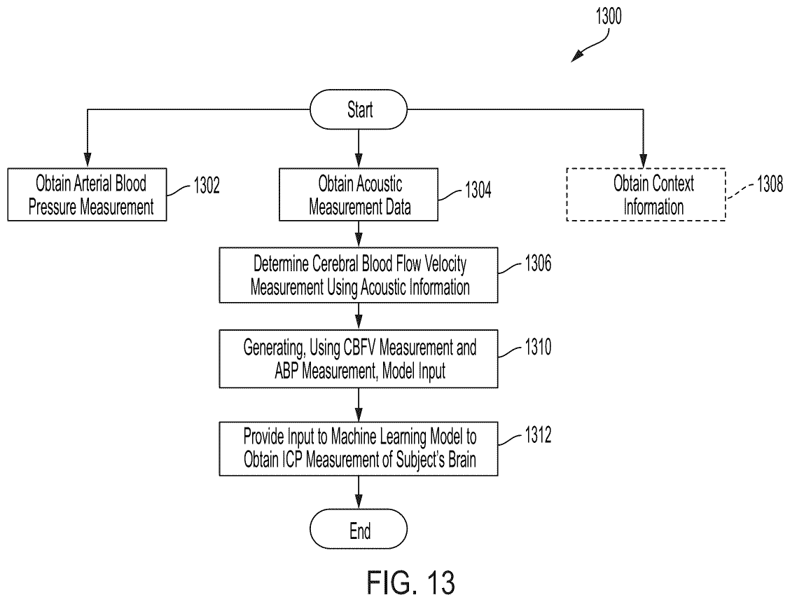

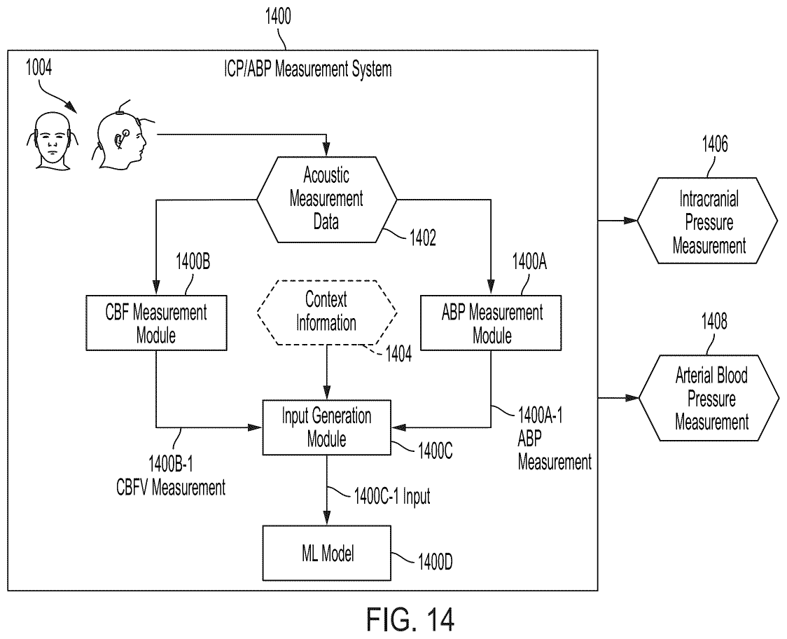

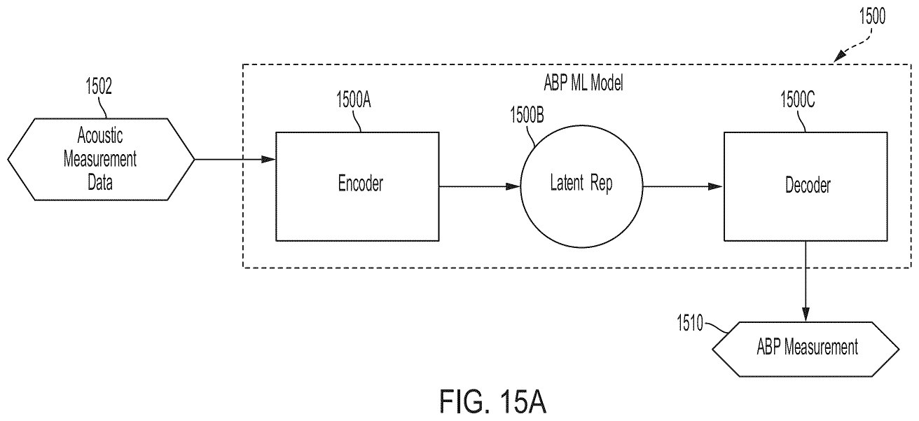

The ABP measurement module 1400A may be configured to use the acoustic measurement data 1402 to determine an ABP measurement 1400A-1 of the subject's brain. In some embodiments, the ABP measurement module 1400A may be configured to determine the ABP measurement 1400A-1 using a ML model. Example ML models are described herein with reference to FIGS. 15A-25C. The ABP measurement module 1400A may be configured to use the acoustic measurement data 1402 to generate input to the ML model. The ABP measurement module 1400A may be configured to provide the input as input to the ML model to obtain the ABP measurement 1400A-1. Example machine learning models for determining the ABP measurement 1400A-1 are described herein with reference to FIG. 15A-15C.

In some embodiments, the ABP measurement 1400A-1 may be a set of ABP measurements in a period of time (e.g., a time series of ABP values). For example, the ABP measurement 1400A-1 may be a waveform of ABP values over the period of time. In some embodiments, the ABP measurement module 1400A may be configured to output the ABP measurement 1400B-1 periodically. For example, the ABP measurement module 1400A may output an ABP measurement every 1 second, 10 seconds, 30 seconds, 1 minute, 2 minutes, 5 minutes, 10 minutes, or other frequency. In some embodiments, the ABP measurement module 1400A may be configured to output the ABP measurement 1400A-1 in response to receiving one or more inputs (e.g., acoustic measurement data 1402 input, and/or context information 1404 input).

In some embodiments, the CBF measurement module 1400B may be configured to use the acoustic measurement data 1402 to determine a CBF measurement of a subject's brain. The CBF measurement module 1400B may be configured to determine a CBF measurement (e.g., CBFV measurement 1400B-1) as described with reference to CBF measurement module 1100A described herein with reference to FIG. 11. The ABP measurement 1408 output by the system 1400 may be the ABP measurement 1400B-1 determined by the ABP measurement module 1400A.

In some embodiments, the input generation module 1400C may be configured generate input 1400C-1 to the ML model 1400D to obtain the ICP measurement 1406 for the subject. The input generation module 1400C may generate input as described herein with reference to input generation module 1100B of FIG. 11. The ML model 1400D may be the ML model 1100C described herein with reference to FIG. 11. Example ML models for determining an ICP measurement of a subject are described herein.

FIG. 15A shows an example ML model 1500 that may be used in obtaining an ABP measurement 1510, according to some embodiments of the technology described herein. The ABP ML model 1500 may be used by the ABP measurement module 1400A of the ICP/ABP measurement system 1400 described herein with reference to FIG. 14.

As shown in the example embodiment of FIG. 15, the ABP ML model 1500 may be an autoencoder including an encoder 1500A and a decoder 1500C. The encoder 1500A is trained to use input generated using acoustic measurement data 1502 to generate a latent representation 1500B. The decoder 1500C is trained to use input generated using the latent representation 1500B to determine an output indicating the ABP measurement 1510. In some embodiments, the encoder 1500A may be trained to use input generated using context information 1504 in addition to the acoustic measurement data 1502 to generate the latent representation 1500B. However, in some embodiments, as indicated by the dotted line of the context information 1504, the ABP ML model 1500 may not use input generated using context information 1504 to generate the latent representation 1500B.

In some embodiments, the latent representation 1500B may be a set of values (e.g., a vector of values) encoding features representing ABP of a subject's brain determined using the acoustic measurement data 1502. The latent representation 1500B may be used to generate input to the decoder 1500C to obtain the ABP measurement 1510. For example, the ICP/ABP measurement system 1400 may be configured to provide the latent representation 1500B as input to the decoder 1500C. In some embodiments, the latent representation 1500B may indicate a probability distribution representing the ABP of the subject's brain (e.g., mean and variance vectors of a Gaussian distribution). The ICP/ABP measurement system 1400 may be configured to obtain one or more samples from the probability distribution as the input to the decoder 1500C. The ICP/ABP measurement system 1400 may be configured to provide the sample(s) as input to the decoder 1500C to obtain one or more ABP values as the ABP measurement 1510.

In some embodiments, the ABP ML model 1500 may be trained using a supervised learning technique. The ABP ML model 1500 may be trained by: (1) obtaining training data including samples of acoustic measurement data and context information, and corresponding ABP measurements; and (2) training the model 1500 by applying a supervised learning technique to the training data. For example, the ABP ML model 1500 may be trained using stochastic gradient descent in which the parameters of the model are iteratively updated to minimize a loss function (e.g., mean squared error, L2 loss, quadratic loss, and/or mean absolute error). In each iteration, a training system may: (1) use an input sample from the training data to obtain an output ABP measurement; and (2) update parameters of the model based on a difference between the output ABP measurement and the ABP measurement corresponding to the input sample from the training data. The training system may iterate until a maximum number of iterations are performed and/or until a threshold value of the loss function is achieved. In some embodiments, the ABP ML model 1500 may be trained by applying a semi-supervised learning technique to the training data.

In some embodiments, the ABP ML model 1500 may be trained in conjunction with an ML model for determining an ICP measurement. The ABP ML model 1500 may be used to generate ABP measurement samples that are used to train the ICP measurement ML model. For example, a training system may sample a probability distribution indicated by the latent representation 1500B, and provide the sample as input to the decoder 1500C to obtain an ABP measurement samples which may be used as input in the ICP measurement ML model. A training system may perform stochastic gradient descent in which the training system updates parameters of the ABP ML model 1500, and parameters of the ICP measurement ML model. In some embodiments, the ABP ML model 1500 may be trained separately from the ICP measurement ML model. For example, the ABP ML model 1500 may be trained using a first set of training data comprising acoustic measurement inputs samples and corresponding ABP measurements, and the ICP measurement ML model may be trained separately using training data comprising input data samples and corresponding ICP measurements.

FIG. 15B shows another example ML model 1520 that may be used to obtain an ABF measurement, according to some embodiments of the technology described herein. In some embodiments, the ML model 1520 may be used by the ABP measurement module 1400A described herein with reference to FIG. 14 to determine the ABP measurement 1400A-1.

As shown in FIG. 15B, the ML model 1520 is a neural network model 1524. The neural network model 1524 receives, as input, a measurement of CBFV 1522 (e.g., CBFV measurement 1400B-1 described herein with reference to FIG. 14). As shown in FIG. 15B, in some embodiments, the measurement of ABP outputted by the neural network model 1524 may include a full-wave APB signal 1526 (e.g., a time series of ABP signal values). In some embodiments, the measurement of ABP may include a differentiated full-wave ABP signal 1528 (e.g., a time series of differentiated ABP values).

FIG. 15C shows another example ML model 1530 that may be used to obtain an ABF measurement, according to some embodiments of the technology described herein. In some embodiments, the ML model 1530 may be used by the ABP measurement module 1400A described herein with reference to FIG. 14 to determine the ABP measurement 1400A-1.

As shown in FIG. 15C, the ML model 1530 is a neural network model 1534. The neural network model 1534 receives, as input, a measurement of CBFV 1532 (e.g., CBFV measurement 1400B-1 described herein with reference to FIG. 14). As shown in FIG. 15C, in some embodiments, the ABP measurement outputted by the neural network model 1534 may include a normalized full-wave APB signal 1536 (e.g., a time series of ABP signal values). In some embodiments, the ABP measurement may include a differentiated full-wave ABP signal 1538 (e.g., a time series of differentiated ABP values). In some embodiments, the ABP measurement may include a minimum and/or maximum 1540 ABP valuc.

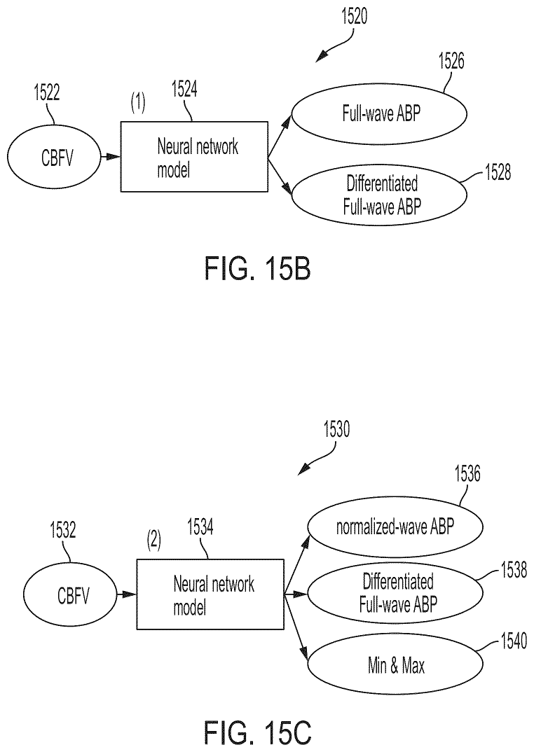

FIG. 16 shows an example process 1600 that may be performed to determine an ABP and ICP measurement of a subject, according to some embodiments of the technology described herein. In some embodiments, process 1600 may be performed by the ICP/ABP measurement system 1400 described herein with reference to FIG. 14.

At block 1602, the system obtains acoustic measurement data. The system may be configured to obtain the acoustic measurement data as described at block 1304 of process 1300 described herein with reference to FIG. 13.

At block 1604, where the system obtains context information. The system may be configured to obtain the context information as described at block 1308 of process 1300. As indicated by the dotted lines of block 1604, in some embodiments, the system may not obtain context information and proceed to block 1606.

At block 1606, the system determines an ABP measurement using an ABP ML model (e.g., ABP ML model 1500, 1520, or 1530 described herein with reference to FIGS. 15A-15C). In some embodiments, the system may be configured generate an input using the acoustic measurement data. For example, the system may determine the input to be a detected acoustic signal (e.g., from performing SBS) of the acoustic measurement data. The system may provide the input to the ML model to obtain an ABP measurement (e.g., a full-wave ABP signal). In some embodiments, the system may be configured to generate input to the ABP ML model using context information in addition to the acoustic measurement data. For example, the system may generate input to the ABP ML model comprising a vector of acoustic signal measurements (e.g., intensity values over a period of time) and context information.

At block 1608, the system determines an ICP measurement using an ML model. In some embodiments, the system may be configured to determine the ICP measurement by performing steps described at blocks 1306, 1310, 1312 of process 1300 described herein with reference to FIG. 13.

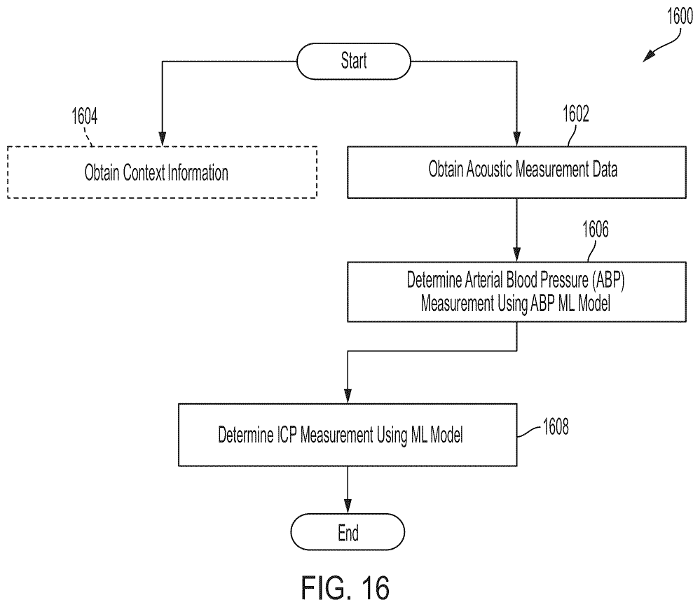

FIG. 17 shows an example ICP measurement system 1700, according to some embodiments of the technology described herein. As shown in FIG. 17, the ICP measurement system 1700 includes probes 1004, a brain perfusion estimator 1700A, an input generation module 1700B, and an ML model 1700C.

In some embodiments, the acoustic measurement data 1702 may be acoustic measurement data 1102 described herein with reference to FIG. 11. The acoustic measurement data 1702 may be obtained using probes 1004. For example, the acoustic measurement data 1702 may be data obtained from perform TCD ultrasound. In some embodiments, the data may be performed by using SBS in conjunction with TCD ultrasound to obtain the acoustic measurement data 1702.

The pulsatility data 1704 may include data about motion of a subject's brain tissue. In some embodiments, the pulsatility data 1704 may be obtained non-invasively. The pulsatility data 1704 may be obtained by transmitting an acoustic signal to a region of the subject's brain, and determining a measure of brain tissue motion by analyzing a subsequent signal from the region of the subject's brain (e.g., by filtering the subsequent signal). Techniques for obtaining pulsatility data 1704 are described herein. The techniques may be referred herein as “pulsatility mode” or “p-mode” sensing.

In some embodiments, the acoustic measurement data 1702 and the pulsatility data 1704 may be obtained using microbubbles. The microbubbles may improve visualization and/or localization of arteries, arterioles, and capillaries. For example, the microbubbles may be contrast agents that increase echogenicity, and thus enhance acoustic contrast and signal to noise ratio (SNR) in the brain. This may facilitate identification of arteries in the brain (e.g., for TCD measurements) and improve accuracy of an estimated measure of brain perfusion from the pulsatility data 1702. Prior to measurement, microbubbles may be injected intravenously. As the microbubbles are being circulated through the vascular system, the concentration of the microbubbles monitored at a target area during a period of time. For example, the microbubbles may be timed and monitored at the target area during the period of time using ultrasound gray-scale (e.g., a B-mode image), color-flow (CFI), or power-doppler images. By measuring acoustic intensity at a region of interest in the period of time, a time intensity curve may be generated. The measurements may be obtained during times at which the acoustic intensity peaks or plateaus as the microbubbles pass through the target region.

The QUS data 1706 may be obtained by transforming scattered acoustic signals into the frequency domain using a Fourier transform prior to B-mode processing. The frequency dependence of the scattered acoustic signals may be related to structure properties of brain tissue. The QUS data 1706 may comprise QUS images. The QUS data 1706 may include an estimation of spectral features of backscattered ultrasound signals, estimation of ultrasonic attenuation and sound speed, parameterization of statistics of an envelope of backscattered ultrasound, ultrasound elastography, ultrasound microscopy, and/or ultrasound computed tomography. Quantitative parameters determined from ultrasonic signals may provide a source of image contrast, which may improve sensitivity of ultrasound. In some embodiments, the QUS data 1706 may be obtained using SBS techniques. The acoustic signals obtained from the SBS techniques may be analyzed to determine a distribution of power as a function of frequency in the acoustic signals.

The ABP measurement 1708 may be ABP measurement 1104 described herein in FIG. 11. As indicated by the dotted lines of the ABP measurement 1708, in some embodiments, the ICP measurement system 1700 may not obtain the ABP measurement 1708 as input. For example, the ICP measurement system 1700 may determine the ABP measurement using acoustic measurement data (e.g., as described herein with reference to FIG. 14 and FIG. 16).

The context information 1710 may be context information 1106 described herein with reference to FIG. 11. Examples of context information are described herein.

As indicated by the dotted lines, in some embodiments, the ICP measurement system 1700 may not obtain context information 1710.

The modules of ICP measurement system 1700 may be implemented using any suitable computing device. For example, the ICP measurement system 1700 may be a desktop computer, a laptop, smartphone, tablet, or other suitable computing device. In some embodiments, the ICP measurement system 1700 may be embedded in an AEG device (e.g., AEG device 1000 described herein with reference to FIGS. 10A-D). For example, the AEG device may be implemented on an embedded circuit within the AEG device. In another example, the AEG device may be implemented using a processor and memory of the AEG device. In some embodiments, the ICP measurement system 1700 may be a cloud based system (e.g., cloud system 1032 described herein with reference to FIG. 10C).

In some embodiments, the brain perfusion estimator 1700A may be configured to use the pulsatility data 1704 and the QUS data 1706 to determine a measurement of brain perfusion 1700A-1. The brain perfusion estimator 1700A may be configured to combine the pulsatility data 1704 with the QUS data 1706 to obtain the measurement of brain perfusion 1700A-1. For example, the brain perfusion estimator 1700A may use the QUS data 1706 to quantify speckle intensity in a p-mode image. The speckle intensity may be quantitatively characterized as the backscattered coefficient.

The ML model 1700C may be trained to generate output indicating the ICP measurement 1712. For example, the ML model 1700C may be a physics guided (e.g., ML model 1200 described herein with reference to FIG. 12A augmented with a model for determining measure of brain perfusion). In some embodiments, the ML model 1700C may include an ML (e.g., a neural network, encoder) trained to receive as input a measure of brain perfusion to generate output representing cerebral perfusion pressure in the subject's brain. The output of the additional ML may be used to provide another input to an ICP estimator. For example, the output of the ML may be provided as input in addition to ABP and/or CBFV based inputs. As another example, the output of the ML model may indicate a probability distribution that may be sampled to obtain input for the ICP estimator.

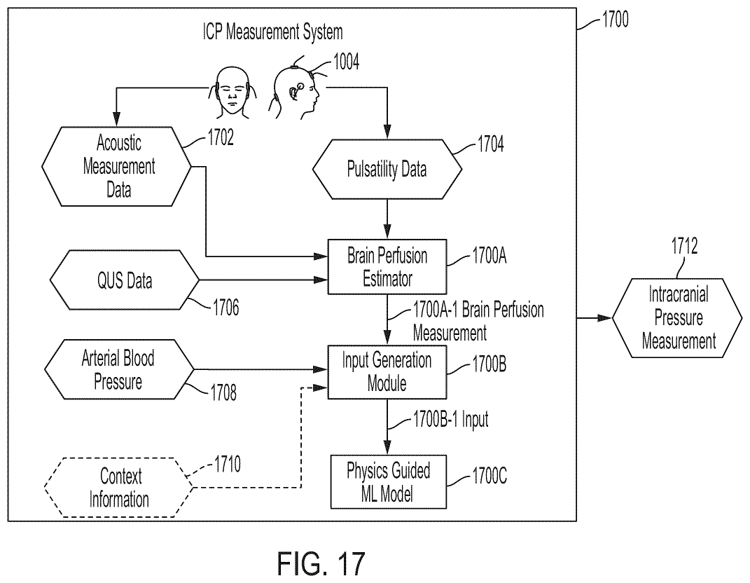

FIG. 18 shows an example process 1800 that may be performed by the ICP measurement system 1700 of FIG. 17 to determine an ICP measurement of a subject's brain, according to some embodiments of the technology described herein.

At block 1802, the system obtains QUS data 1802 for a subject's brain. In some embodiments, the system may be configured to obtain the QUS data 1802 using SBS and ultrasound. For example, the system may process acoustic signals detected from application of ultrasound signals during SBS to obtain the QUS data 1802.

At block 1804, the system obtains pulsatility data. In some embodiments, the system may be configured to obtain the pulsatility data using p-mode sensing. Example techniques of p-mode sensing are described herein.

At block 1808, the system obtains an ABP measurement. In some embodiments, the system may be configured to obtain an ABP measurement as described at block 1304 of process 1300 described herein with reference to FIG. 13. In some embodiments, the system may be configured to obtain an ABP measurement as described at block 1606 of process 1600 described herein with reference to FIG. 16.

At block 1810, where the system obtains context information. In some embodiments, the system may be configured to obtain context information as described at block 1308 of process 1300. As indicated by the dotted, lines, in some embodiments, the process 1800 may not include obtaining of context information.

At block 1806, the system determines a measure of brain perfusion using the QUS data and the pulsatility data. In some embodiments, the system may be configured to quantify brain perfusion as speckle statistics determined using the QUS data and the pulsatility data (e.g., p-mode sensing data).

At block 1812, the system determines an ICP measurement of the subject's brain using an ML model (e.g., a physics guided ML model). In some embodiments, the system may be configured to use the measure of brain perfusion (e.g., perfusion speckle statistics) to generate input to a ML model (e.g., neural network). The system may provide the measure of brain perfusion as input to the ML model to obtain a representation of the brain perfusion. The system may be configured to provide the representation of the brain perfusion as input to an ICP estimator model (e.g., with other inputs) to obtain the ICP measurement for a subject.

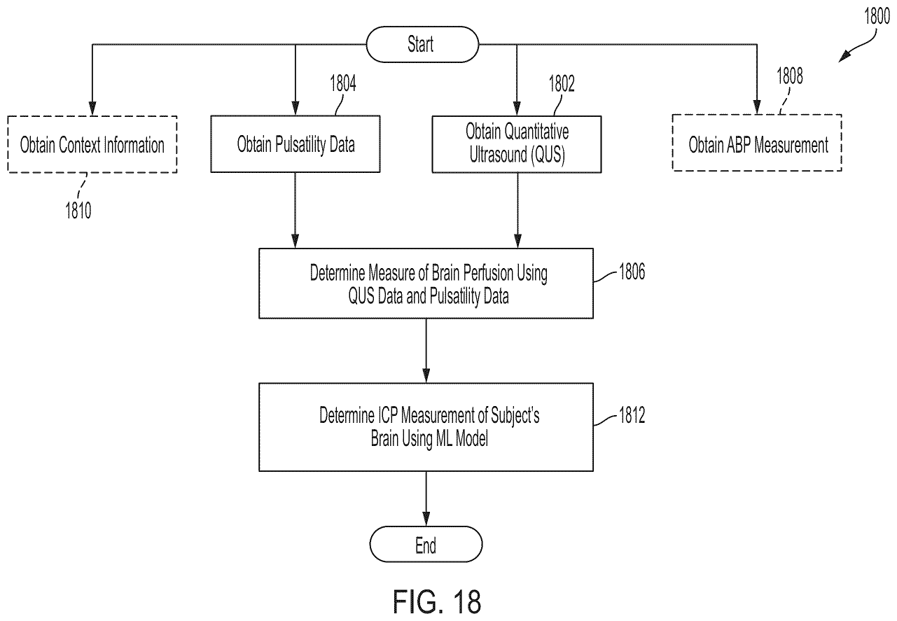

FIG. 19 shows an example ICP measurement system 1900, according to some embodiments of the technology described herein. The ICP measurement system 1900 includes probes 1004, a ventricle deformation measurement module 1900A, input generation module 1900B, and an ML model 1900C.

As shown in FIG. 19, the ICP measurement system 1900 obtains as input acoustic measurement data 1902 and context information 1904, and determines an ICP measurement 1906. The acoustic measurement data 1902 may be acoustic measurement data 1102 described herein with reference to FIG. 11. The context information 1904 may be context information 1106 described herein with reference to FIG. 11. As indicate by the dotted lines of the context information 1904, in some embodiments, the ICP measurement system 1900 may not obtain the context information 1904 as input.

In some embodiments, the ventricle deformation measurement component 1900A may be configured to determine a ventricle deformation measurement 1900A-1 of the subject's brain. In some embodiments, the ventricle deformation measurement component 1900A may be configured to determine the ventricle deformation measurement 1900A-1 using the acoustic measurement data 1902. For example, the acoustic measurement 1900A-1 may be obtained from performing SBS. In some embodiments, the ventricle deformation measurement component 1900A may be configured to determine the ventricle deformation measurement 1900A-1 using p-mode sensing techniques described herein.

The ventricle deformation measurement component 1900A may be configured to determine the ventricle deformation measurement 1900A-1 by determining a measurement of contraction and expansion of a ventricle in the subject's brain during one or more cardiac cycles. The ventricle deformation measurement component 1900A may be configured to determine a change in dimensions of a ventricle over a period of time of the cardiac cycle(s). For example, the ventricle deformation measurement component 1900A may determine a change in distance between ventricle walls over a period of time. In another example, the ventricle deformation measurement component 1900A may determine a change in surface area of a ventricle over a period of time. In another example, the ventricle deformation measurement component 1900A may determine a change in volume of a ventricle over a period of time.

In some embodiments, the ML model 1900C may be a physics model on an elasticity model of the subject's brain. For example, the ML model 1900C may be a physics guided model based on the Saint Venant-Kirchhoff model of the brain. In the Venant-Kirchhoff model, the energy of the system may be approximated by equation 4 below.

$$W ( E ) = {\frac{α}{2} [ tr ( E ) ]}^{2} + μ tr ( {E}^{2} ) \qquad Equation ( 4 )$$

In equation 4 above, E indicates strain. The strain may be estimated by equation 5 below.

$${E = \frac{1}{2} [ {∇}_{x} u )}^{T} + {∇}_{x} u + {( {∇}_{x} u )}^{T} \cdot {∇}_{x} u ] \qquad Equation ( 5 )$$

The pressure may be approximated by equation 6 below.

$$S = \frac{\partial W}{\partial E} = α t r ( E ) I + 2 μE \qquad Equation ( 6 )$$

In equations 4-6 above, a, u are elasticity parameters that can be fitted by collecting pressure measurements.

Although the Saint Venant-Kirchhoff model is described herein as an example of the physics model 1900B, in some embodiments, the ML model 1900C may be guided based on a different physics model. For example, the ML model 1900C may be guided by the Fung model, Mooney-Rivlin model, Ogden model, polynomial model, Yeoh model, Marlow model, Arruda-Boyce model, Neo-Hookean model, Buche-Silberstein model, and/or another suitable model. In some embodiments, the model may include viscoelastic model. In some embodiments, the ML model 1900C may be a single ML model. For example, the ML model 1900C may be a single neural network. The ML model 1900C may be trained using ventricle deformation measurements and corresponding ICP measurements (e.g., by applying a supervised learning technique).

In some embodiments, the input generation module 1900B may be configured to generate input 1900B-1 using the ventricle deformation measurement 1900A-1. The input generation module 1900B may provide the input 1900B-1 to the ML model 1900C to obtain an ICP measurement 1906 for a subject.

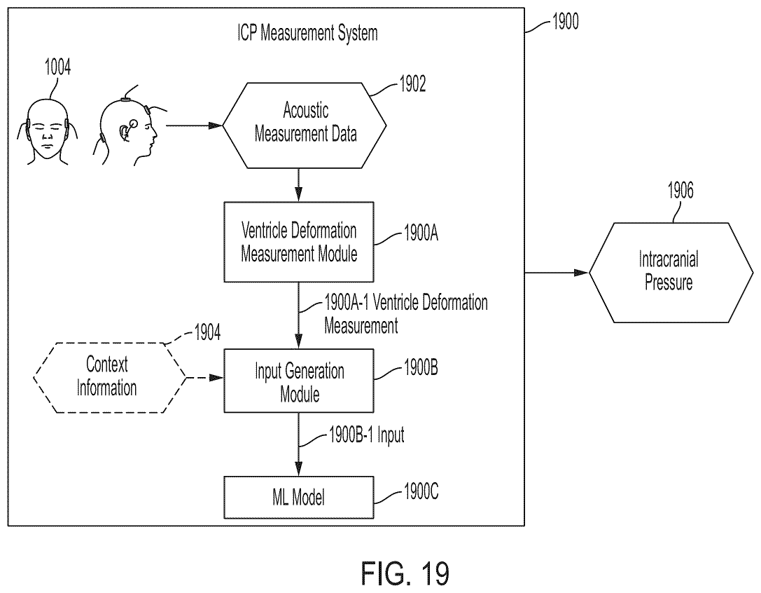

FIG. 20 shows an example process 2000 for determining an ICP measurement for a subject, according to some embodiments of the technology described herein. In some embodiments, process 2000 may be performed by the ICP measurement system 1900 of FIG. 19.

At block 2002, the system obtains acoustic measurement data 2002. In some embodiments, the acoustic measurement data may be obtained using SBS. For example, an acoustic beam may be guided towards a region of interest in a subject's brain, and a responsive acoustic signal may be detected to obtain the acoustic measurement data. In some embodiments, the system may be configured to obtain the acoustic measurement data as described at block 1304 of process 1300 described herein with reference to FIG. 13.

At block 2004, the system determines a ventricle deformation measurement using the acoustic measurement data. In some embodiments, the system may be configured to determine the ventricle deformation measurement using p-mode sensing techniques. The system may be configured to use p-mode sensing to track and measure ventricle contraction and expansion over a cardiac cycle (“beat”). For example, the system may determine a distance between ventricle walls (e.g., in image pixels, or in millimeters), surface area of the ventricle (e.g., in image pixels, or millimeters squared), and/or volume of the ventricle over the cardiac cycle (e.g., in millimeters cubed).

At block 2006, the system obtains context information. The system may be configured to obtain context information as described at block 1308 of process 1300 described herein with reference to FIG. 13. As indicated by the dotted lines of block 2006, in some embodiments, the system may not obtain context information.

At block 2008, the system determines an ICP measurement using an ML model (e.g., ML model 1900C). In some embodiments, the system may be configured to generate one or more inputs for the ML model to obtain representation(s) of respective aspect(s) of the brain, and provide the representation(s) as input to an ICP estimator ML model to obtain output indicating the ICP measurement. In some embodiments, the system may be configured to use equations of a model to determine the ICP measurement. For example, the system may use one or more values output by ML model(s) of the ML model to calculate the ICP measurement using equations of a model (e.g., equations 4-6). In some embodiments, the ML model may be a single ML model (e.g., a neural network). The system may be configured to: (1) generate input for the ML model using the ventricle deformation measurement and, optionally, context information 1904; and (2) provide the input to the ML model to obtain output indicating the ICP measurement.

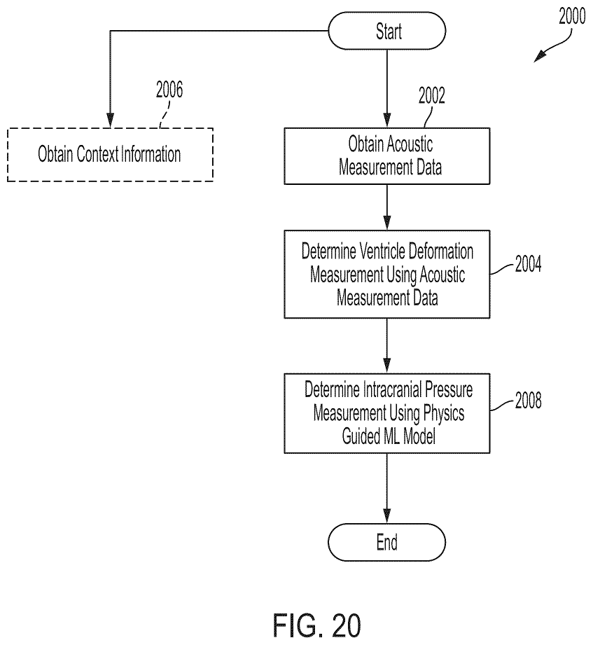

FIG. 21 shows an example ABP measurement system 2100, according to some embodiments of the technology described herein. The ABP measurement system 2100 includes an arterial deformation measurement module 2100A, an input generation module 2100B, and an ML model 2100C.

As shown in FIG. 21, the ABP measurement system 2100 obtains as input acoustic measurement data 2102 and context information 2104, and determines an ABP measurement 2106. The acoustic measurement data 2102 may be obtained from using SBS techniques described herein. The context information 2104 may be context information 1106 described herein with reference to FIG. 11. As indicate by the dotted lines of the context information 2104, in some embodiments, the ABP measurement system 2100 may not obtain the context information 2104 as input.

In some embodiments, the arterial deformation measurement component 2100A may be configured to determine an arterial wall deformation measurement 2100A-1 of the subject's brain. In some embodiments, the arterial deformation measurement component 2100A2100A-1 may be configured to determine the arterial wall deformation measurement 2100A-1 using the acoustic measurement data 2102. In some embodiments, the ventricle deformation measurement component 2100A may be configured to determine the ventricle deformation measurement 2100A-1 using the p-mode sensing techniques described herein.

In some embodiments, the arterial deformation measurement component 2100A may be configured to determine the arterial wall deformation measurement 2100A-1 by determining a change in dimensions of an artery over a period of time. In some embodiments, the arterial deformation measurement component 2100A may be configured to determine the change in dimensions of an artery over a cardiac cycle. For example, the arterial deformation measurement component 2100A may determine a change in distance between artery walls over a period of time. In another example, the arterial deformation measurement component 2100A may determine a change in cross-sectional surface area of an artery over a period of time. In another example, the article deformation measurement component 2100A may determine a change in volume of a portion of an artery over a period of time.

In some embodiments, the input generation module 2100B may be configured to use an arterial wall deformation measurement 2100A-1 to generate input 2100B-1 for the ML model 2100C to obtain an output indicating the ABP measurement 2106. The input generation module 2100B may provide the input 2100B-1 to the ML model 2100C to obtain the ABP measurement 2106.

In some embodiments, the ML model 2100C may be a physics guided model based on an elasticity model of the subject's brain. Example models are described herein with reference to FIG. 19. The ABP measurement system 2100 may be configured to use the ML model 2100C to determine the ABP measurement 2106. In some embodiments, the ML model 2100C may include one or more ML models (e.g., neural network(s)). For example, the ML model 2100C may be trained using arterial wall deformation measurements and corresponding ABP measurement values (e.g., by applying a supervised learning technique to a training data set). In some embodiments, the ML model 2100C may be a single ML model. For example, the ML model 2100C may be a single neural network. The ML model 2100C may be trained using ventricle deformation measurements and corresponding ABP measurements (e.g., by applying a supervised learning technique).

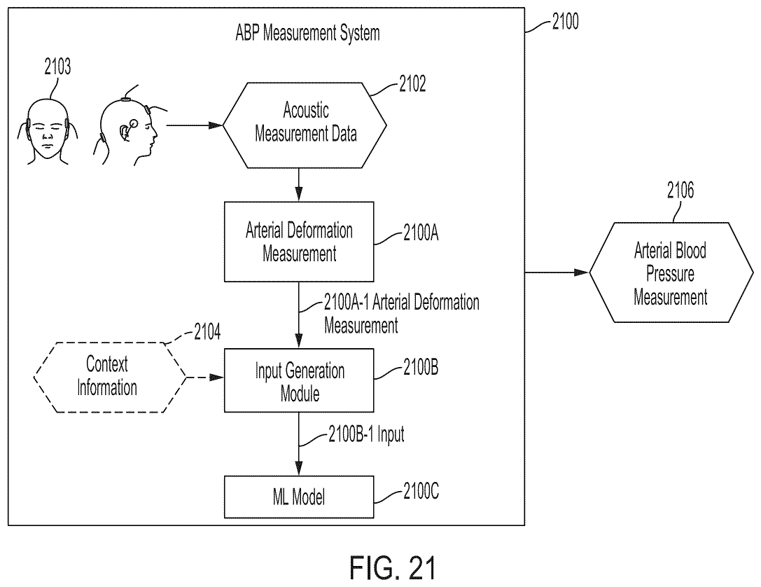

FIG. 22 shows an example process 2200 for determining an ABP measurement for a subject, according to some embodiments of the technology described herein. In some embodiments, process 2200 may be performed by the ABP measurement system 2100 of FIG. 21 to determine an ABP measurement of a subject's brain.

At block 2202, the system obtains acoustic measurement data 2202. In some embodiments, the acoustic measurement data may be obtained using SBS. For example, an acoustic beam may be guided towards a region of interest in a subject's brain, and a responsive acoustic signal may be detected to obtain the acoustic measurement data. In some embodiments, the system may be configured to obtain the acoustic measurement data as described at block 1304 of process 1300 described herein with reference to FIG. 13.

At block 2204, the system obtains context information. The system may be configured to obtain context information as described at block 1308 of process 1300 described herein with reference to FIG. 13. As indicated by the dotted lines of block 2206, in some embodiments, the system may not obtain context information.

At block 2206, the system determines an arterial wall deformation measurement using the acoustic measurement data. In some embodiments, the system may be configured to determine the arterial wall deformation measurement using p-mode sensing techniques. The system may be configured to use p-mode sensing to track and measure arterial contraction and expansion over a cardiac cycle (“beat”). For example, the system may determine a distance between artery walls (e.g., in image pixels, or in millimeters), cross-sectional surface area of an artery (e.g., in image pixels, or millimeters squared), and/or volume of a portion of an artery over the cardiac cycle (e.g., in millimeters cubed).

At block 2208, the system determines an ABP measurement using an ML model (e.g., ML model 2100C described herein with reference to FIG. 21). In some embodiments, the system may be configured to generate one or more inputs for the ML model to obtain representation(s) of respective aspect(s) of the brain, and provide the representation(s) as input to an ABP estimator ML model to obtain output indicating the ABP measurement. In some embodiments, the system may be configured to use equations of a model to determine the ABP measurement. For example, the system may use one or more values output by ML model(s) of the physics guided ML model to calculate the ABP measurement using equations of a model (e.g., equations 4-6). In some embodiments, the physics guided ML model 2100B may be a single ML model (e.g., a neural network). The system may be configured to: (1) generate input for the ML model using the arterial deformation measurement and, optionally context information 2104; and (2) provide the input to the ML model to obtain output indicating the ABP measurement.

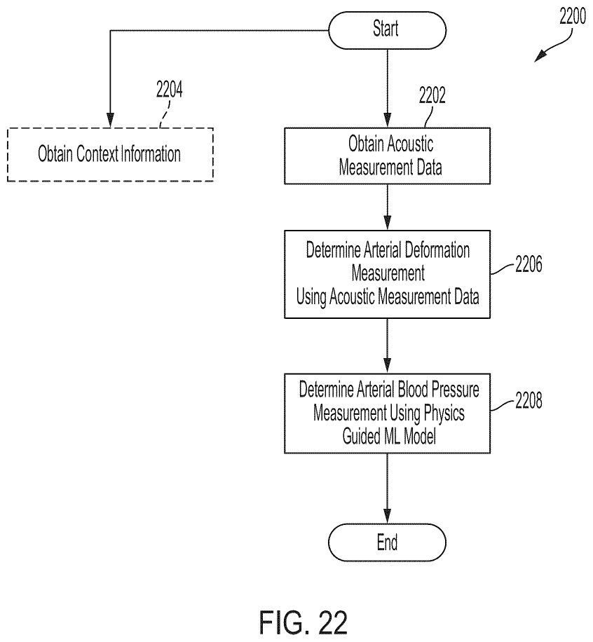

FIG. 23 shows an example arterial elastance measurement system 2300, according to some embodiments of the technology described herein. Arterial elastance measurement system 2300 includes probes 1004, an arterial deformation measurement module 2300A, and an arterial elastance determination module 2300B.

As shown in FIG. 23, the arterial elastance system obtains as input acoustic measurement data 2302 and uses it to determine an arterial elastance measurement 2306. The acoustic measurement data 2302 may be acoustic measurement data 1102 described herein with reference to FIG. 11 (e.g., data obtained using SBS).

In some embodiments, the arterial measurement component 2300A may be configured to track two or more points at different arterial points in the brain using the acoustic measurement data 2302 to determine an arterial deformation measurement 2300A-1. For example, it may track two or more points on an artery of the Circle of Willis. The arterial measurement component 2300A may be configured to determine a pulse wave velocity (PWV), which indicates a velocity at which blood pressure propagates through the circulatory system over a period of time (e.g., over a cardiac cycle). The arterial deformation measurement component 2300A may be configured to determine the PWV by: (1) determining a pulse transit time (PTT), which is the time it takes for a pressure wave to go from upstream pressure to downstream pressure; (2) determining a distance of movement of two or more points; and (3) dividing the distance by the PTT to obtain the PWV.

In some embodiments, the arterial elastance determination module 2300B may be configured to use the arterial deformation measurement 2300A-1 to determine the arterial elastance measurement 2306. For example, the arterial elastance determination module 2300B may use PWV to determine the measure of arterial elastance 2306 of an artery in the subject's brain. For example, the arterial measurement component may use equation 7 below to determine the elasticity.

$$PWV = \sqrt{{E}_{inc} \cdot \frac{h}{2 r ρ}} \qquad Equation ( 7 )$$

In equation 7 above, E_{inc} is the elasticity of the arterial wall, p is the artery density, r is the radius, and h is the wall thickness of the artery.

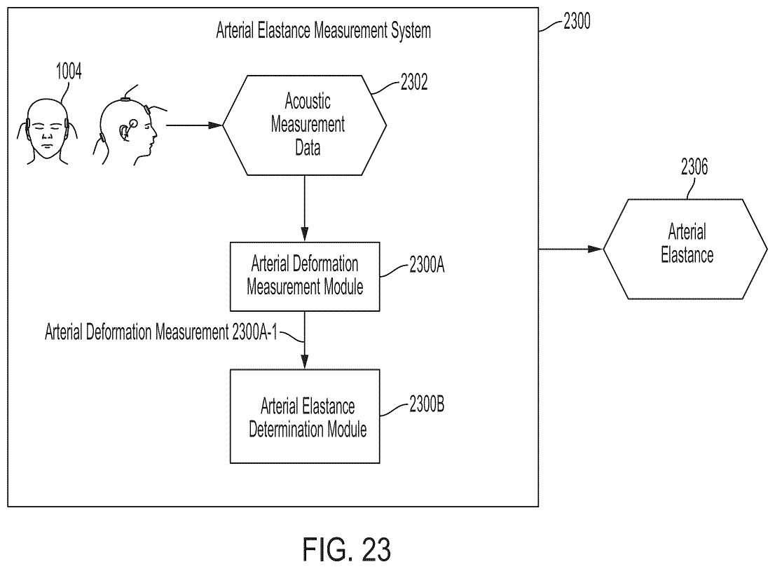

FIG. 24 shows an example process 2400 of determining an arterial elastance measurement of a subject's brain. In some embodiments, the process 2400 may be performed by the arterial elastance measurement system 2300 of FIG. 23.

At block 2402, the system obtains acoustic measurement data. The system may be configured to obtain the acoustic measurement data as described at block 1304 of process 1300 described herein with reference to FIG. 13. For example, the acoustic measurement data may be obtained using SBS. At block 2402, the system determines an arterial deformation measurement using the acoustic measurement data. The system may be configured to determine the arterial deformation measurement by: (1) measuring blood flow; and (2) tracking two or more points at different arterial points. The system may be configured to determine a PWV by: (1) determining a distance that a pressure pulse travels; (2) determining a PTT; and (3) dividing the distance by the PTT. For example, the system may determine the distance by determining a distance between two points on the artery between which a pulse of pressure travelled. The system may be configured to measure dimensions of the artery including a radius, wall thickness, and density.

At block 2408, the system determines an arterial elastance measurement using the arterial deformation measurement. In some embodiments, the system may calculate the elastance using the PWV (e.g., using equation 7). In some embodiments, the system may include a physics guided ML model, and the system may use the arterial deformation measurement to generate one or more inputs for the physics guided ML model to obtain the arterial elastance measurement. In some embodiments, the system may be configured to provide the arterial deformation measurement (e.g., PWV) as input to an ML model (e.g., a neural network) to obtain output indicating the arterial elastance measurement.

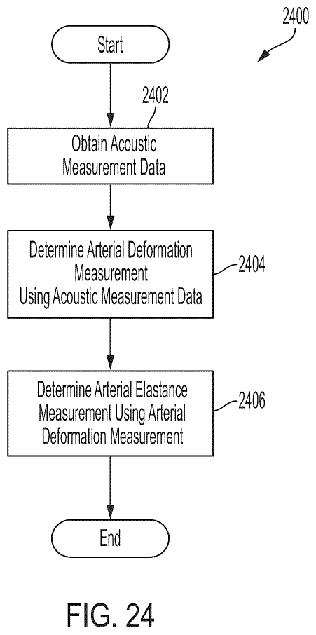

FIG. 25 shows an example brain elasticity measurement system 2500, according to some embodiments of the technology described herein. The brain elasticity system 2500 includes a movement tracking module 2500A and an intercranial elastance measurement module 2500B. As shown in FIG. 25, the brain elasticity measurement system 2500 obtains as input pulsatility data 2502 and uses it to determine a measurement of brain elastance 2506.

The pulsatility data 2502 may be as described with reference to pulsatility data 1704 of FIG. 17. For example, the pulsatility data 2502 may be data obtained from performing p-mode sensing. In some embodiments, the pulsatility data 2502 may include a waveform of movement of one or more tissue areas in brain tissue of a subject's brain.

In some embodiments, the movement tracking component 2500A may be configured to analyze a waveform of tissue area(s) in brain tissue to determine a measurement 2500A-1 of movement of the tissue area(s) in the brain. In some embodiments, the movement tracking component 2500A may be configured to determine a change in phase of the waveform. The movement tracking module 2500A may be configured to provide the tissue movement measurement 2500A-1 to the intracranial elastance measurement module 2500B.

In some embodiments, the intracranial elastance measurement module 2500B may be configured to determine the intracranial elastance measurement 2506 using the tissue movement measurement 2500A-1.

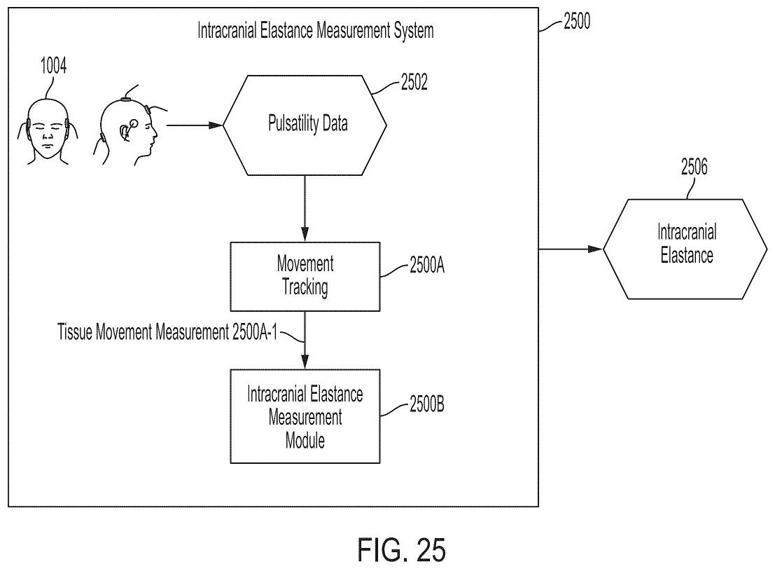

FIG. 26 shows an example process 2600 of determining intracranial elastance of a subject's brain, according to some embodiments of the technology described herein. In some embodiments, the process 2600 may be performed by the brain elasticity measurement system 2500 of FIG. 25.

At block 2602, the system obtains pulsatility data. The system may be configured to obtain pulsatility data as described at block 1804 of process 1800 described herein with reference to FIG. 18.

At block 2604, the system determines a measurement of movement of one or more tissue areas of the subject's brain using the pulsatility data. In some embodiments, the system may be configured to determine the measurement based on a waveform of movement of the tissue area(s). In some embodiments, the system may be configured to determine the measurement based on a phase change of the waveform.

At block 2606, the system determines an elasticity measurement of the subject's brain using the measurement of movement. In some embodiments, the system may be configured to use a physics guided ML model. The system may be configured to generate one or more inputs to the physics guided ML model to obtain output indicating the elasticity measurement. The physics guided ML model may be based on an elasticity model of the brain. Example elasticity models are described herein. In some embodiments, the system may be configured to use an equation of a physics model of elasticity to calculate the elasticity measurement. In some embodiments, the system may be configured to an ML model (e.g., a neural network) trained to output the measurement of elasticity based on an input of the measurement of the movement. The system may provide the measurement of movement as input to the ML model to obtain output indicating the elasticity measurement.

### Smart Beam-Steering Techniques

### System Overview

In some aspects, the beam-steering techniques described herein can be used to autonomously steer acoustic beams (e.g., ultrasound beams) in the brain. The techniques can be used to identify and lock on regions of interest, such as different tissue types, vasculature, and/or physiological abnormalities, while correcting for movements and drifts from the target. The techniques can further be used to sense, detect, diagnose, and monitor brain functions and conditions, such as epileptic seizure, intracranial pressure, vasospasm, and hemorrhage.

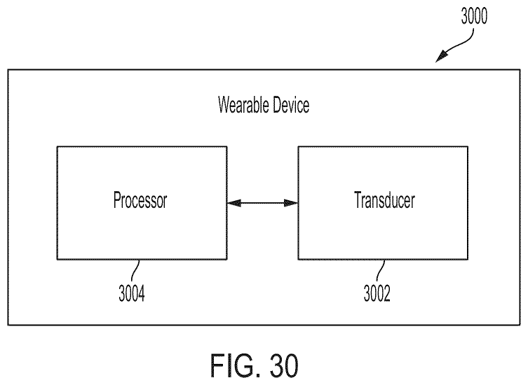

FIG. 30 shows a block diagram for a wearable device 3000 for autonomous beam steering, according to some embodiments of the technology described herein. The device 3000 is wearable by (or attached to or implanted within) a person. The device 3000 includes a transducer 3002 and a processor 3004.

The transducer 3004 may be configured to receive and/or apply to the brain an acoustic signal. In some embodiments, the acoustic signal includes any physical process that involves the propagation of mechanical waves, such as acoustic, sound, ultrasound, and/or elastic waves. In some embodiments, receiving and/or applying to the brain an acoustic signal involves forming a beam and/or utilizing beam-steering techniques, further described herein. In some embodiments, the transducer 3004 may be disposed on the head of the person in a non-invasive manner.

The processor 3004 may be in communication with the transducer 3002. The processor 3004 may be programmed to receive, from the transducer 3002, the acoustic signal detected from the brain and to transmit an instruction to the transducer 3002. In some embodiments, the instruction may indicate a direction for forming a beam for detecting an acoustic signal and/or for applying to the brain an acoustic signal. In some embodiments, the processor 3002 may be programmed to analyze data associated with the acoustic signal to detect and/or localize structures and/or motion in the brain, such as different anatomical landmarks, tissue types, musculature, vasculature, blood flow, brain beating, and/or physiological abnormalities. In some embodiments, the processor 3002 may be programmed to analyze data associated with the acoustic signal to determine a segmentation of different structures in the brain, such as the segmentation of different tissue types and/or vasculature. In some embodiments, the processor 3002 may be programmed to analyze data associated with the acoustic signal to sense and/or monitor brain metrics, such as intracranial pressure, cerebral blood flow, cerebral profusion pressure, and intracranial elastance.

### Beamforming and Beam-Steering

In some embodiments, the transducer (e.g., transducer 3002) may be configured for transmit—and/or receive-beamforming. The transducer may include transducer elements that are each configured to transmit waves (e.g., acoustic, sound, ultrasound, elastic, etc.) in response to being electrically excited by an input pulse. Transmit beamforming involves phasing (or time-delaying) the input pulses with respect to one another, such that waves transmitted by the elements constructively interfere in space and concentrate the wave energy into a narrow beam in space. Receive-beamforming involves reconstructing a beam by synthetically aligning waves that arrive at and are recorded by the transducer elements with different time delays.

In some embodiments, the functions of a processor (e.g., processor 3004) may include generating transmit timing and possible apodization (e.g., weighting, tapering, and shading) during transmit-beamforming, supplying the time delays and signal processing during receive-beamforming, supplying apodization and summing of delayed echoes, and/or additional signal processing-related activities. In some embodiments, it may be desirable to create a narrow and uniform beam with low sidelobes over a long depth. During both transmit and receive operations, appropriate time delays may be supplied to elements of the transducer to accomplish appropriate focusing and steering.

The direction of transmit- and/or receive-beamforming may be changed using beam-steering techniques. For example, the direction for forming a beam (e.g., beamforming) may be changed by changing the set of time-delays applied to the elements of the transducer. Beam-steering may be performed by any suitable transducer, e.g., transducer 3002 to change the direction for forming the beam.

In some embodiments, the beam may be steered in any suitable direction in any suitable order. For example, the beam may be steered left to right, right to left, start at elevation first, and/or start at azimuthal first.

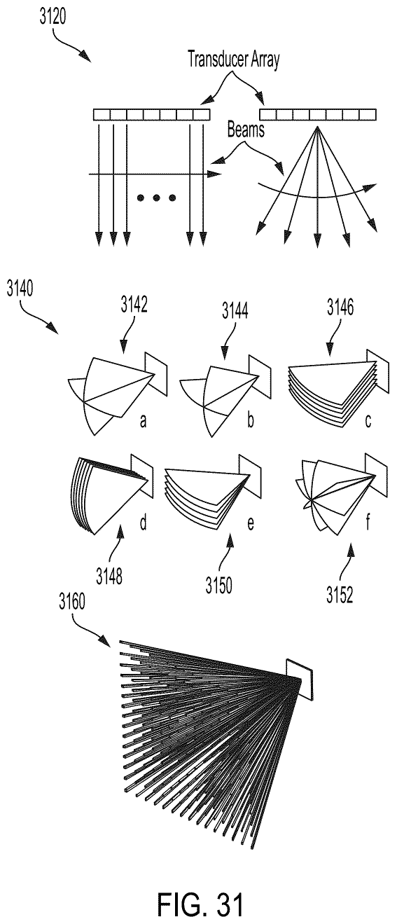

In some embodiments, a transducer consists of multiple transducer elements arranged into an array (e.g., a one-dimensional array or a two-dimensional array). Beam-steering may be conducted by a one-dimensional array over a two-dimensional plane using any suitable architecture. For example, as shown in FIG. 31, a one-dimensional array 3120 may include a linear, curvilinear, and/or phased array. Additionally or alternatively, beam-steering may be conducted by a two-dimensional probe array over a three-dimensional volume using any three-dimensional beam-steering technique. For example, as shown in FIG. 31, three-dimensional beam-steering techniques may include planar 3140, full volume 3160, and random sampling techniques (not shown). Planar beam-steering 3140 may include biplane 3142, biplane with an angular sweep 3144, translational 3146, 3148, tilt 3150, and rotational 3152. In some embodiments, three-dimensional beam-steering may be done via mechanical scanning (e.g., motorized holder or robotic arm) and/or fully electronic scanning along the third dimension.

### Smart-Beam Steering System

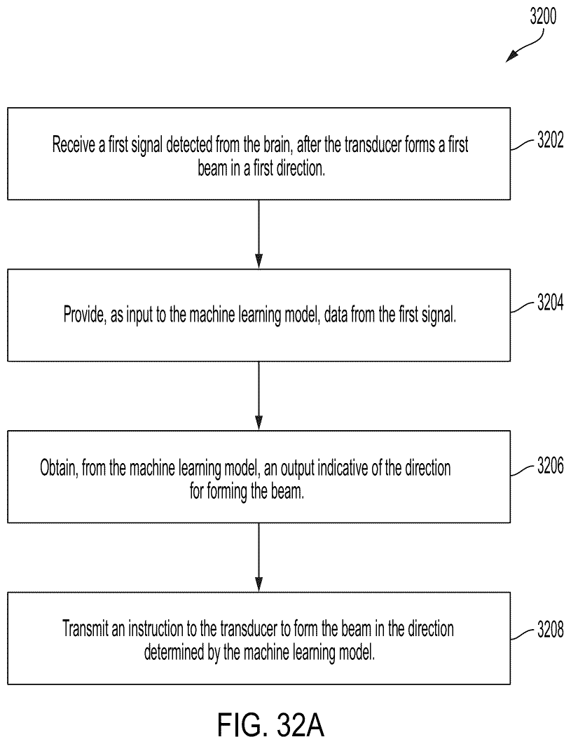

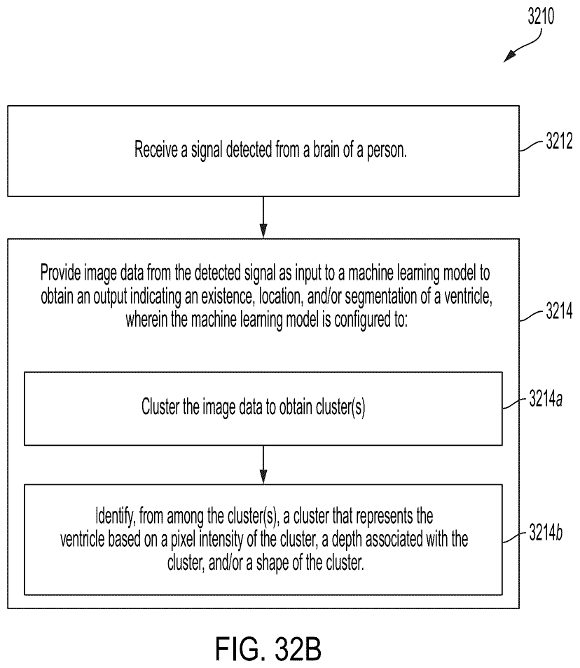

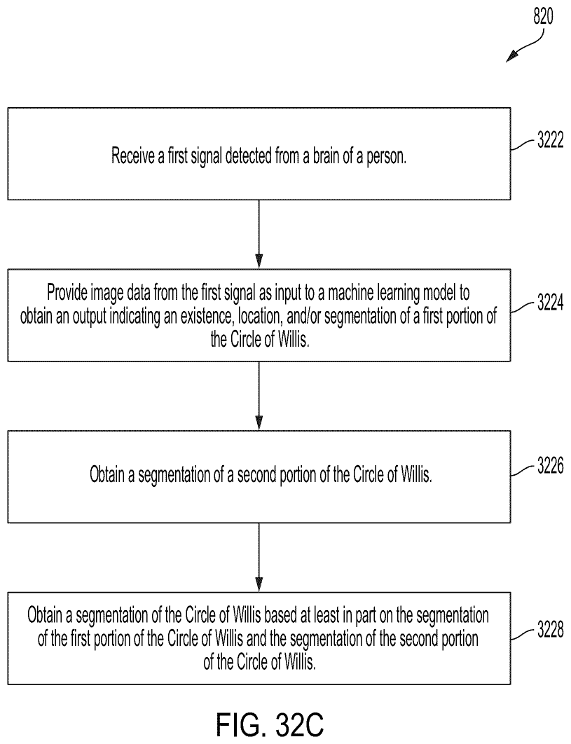

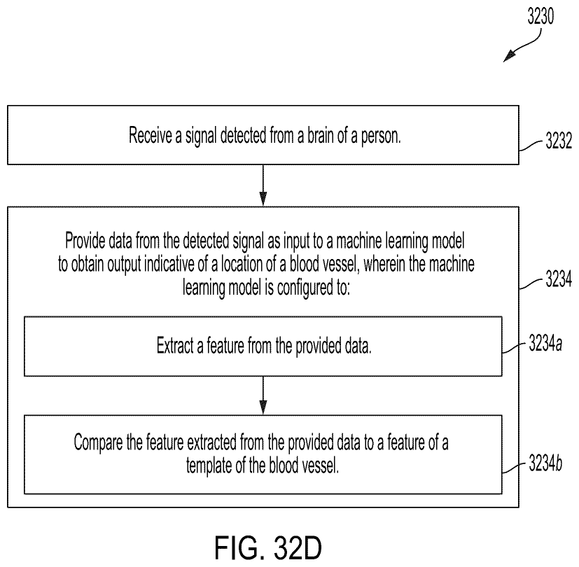

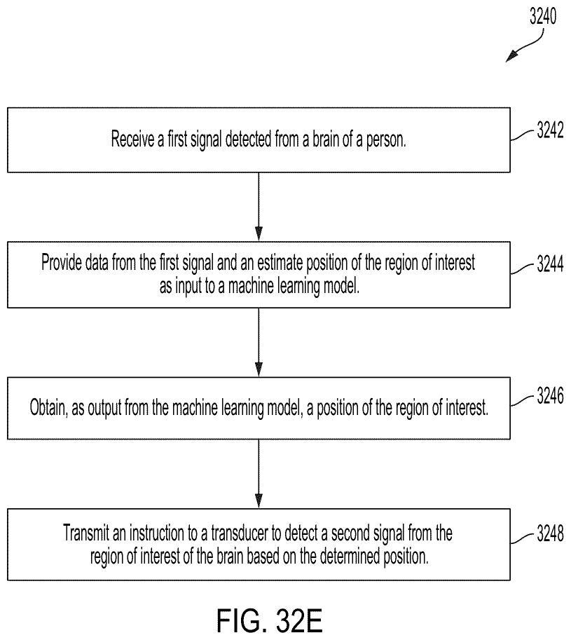

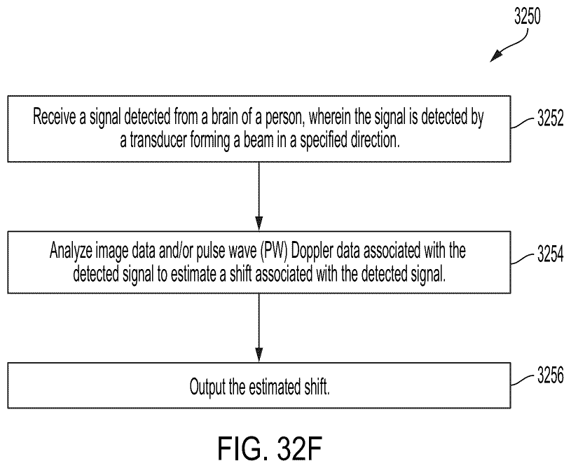

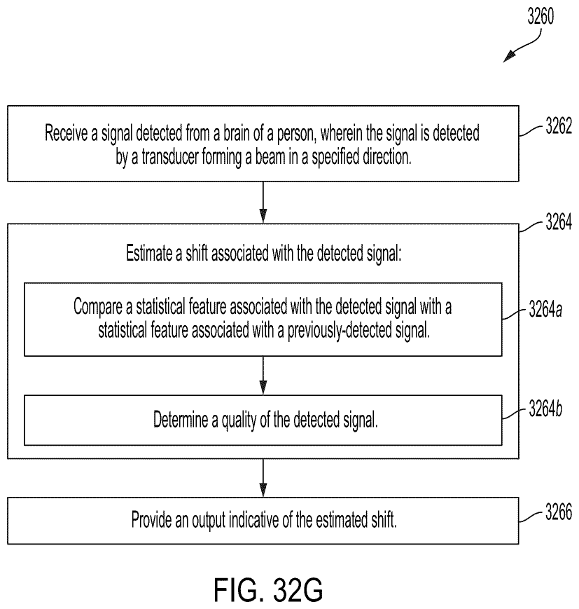

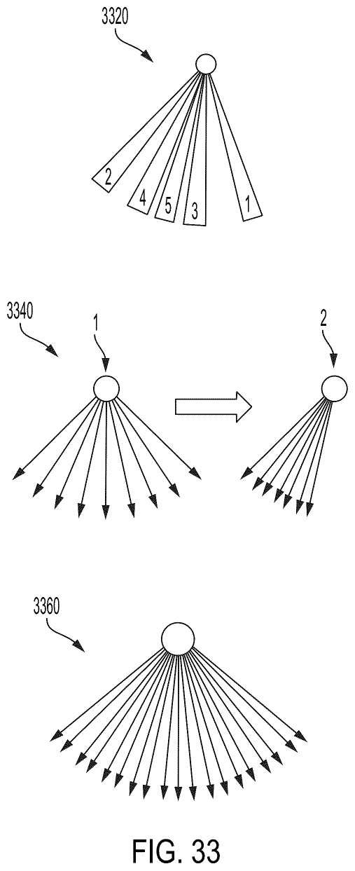

FIG. 32A shows a flow diagram 3200 for a method for autonomous beam-steering, according to some embodiments of the technology described herein. In some embodiments, the method may be implemented using a processor, such as processor 604. In some embodiments, the techniques may be used for autonomously detecting a signal from a region of interest of the brain, examples of which are described herein, including at least with respect to FIG. 33.

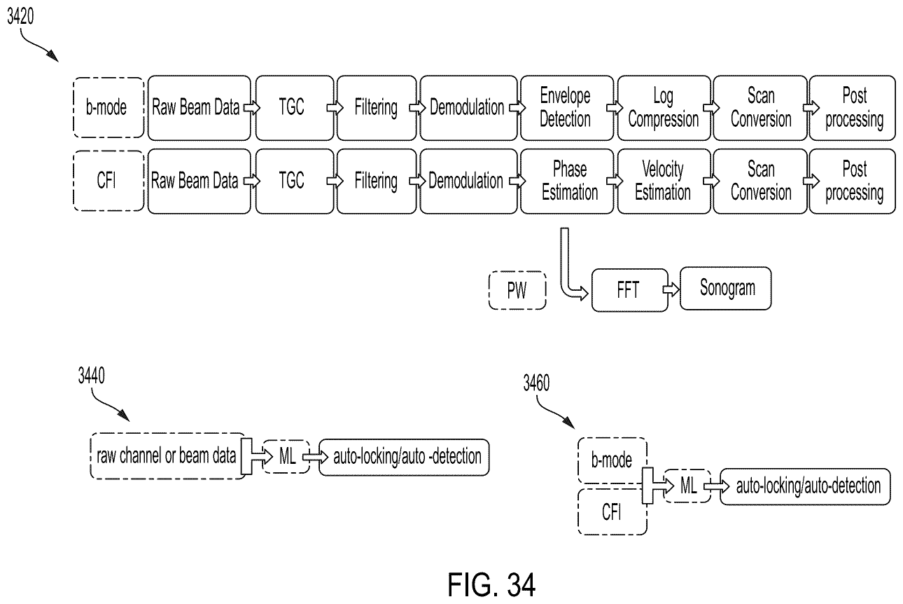

At 3202, the techniques include receiving a first signal detected from the brain. In some embodiments, the transducer detects the signal after forming a first beam (e.g., receive- and/or transmit-beamforming) in a first direction. In some embodiments, the first direction may be a default direction, a direction determined using the techniques described herein including with respect to FIG. 33 and/or a direction previously determined using the machine learning techniques described herein. In some embodiments, data from the first signal includes data acquired from a single acoustic beam, a sequence of acoustic beams over a two-dimensional plane, acoustic beams over a sequence of two-dimensional planes, and/or acoustic beams over a three-dimensional volume. In some embodiments, the data may include raw beam data and/or data acquired as a result of one or more processing techniques, such as the processing techniques described herein including with respect to FIG. 34. In some embodiments, the data may be processed to generate B-mode (brightness mode) imaging data, CFI (color-flow imaging) data, PW (pulse-wave) Doppler data, and/or data resulting from any suitable ultrasound modality.

At 3204, the techniques include providing the data (e.g., raw data and/or processed data) from the first signal as input to a trained machine learning model. At 3206, the trained machine learning model may output the direction, with respect to the brain of a person, for forming the beam to detect the signal from the region of interest.

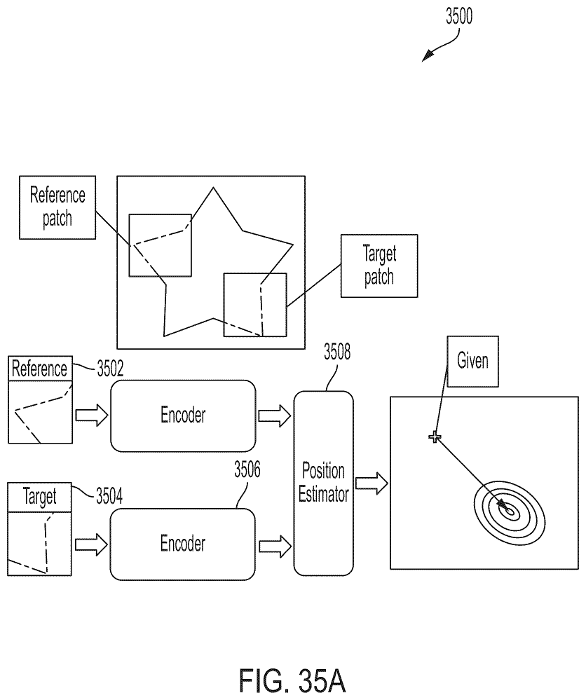

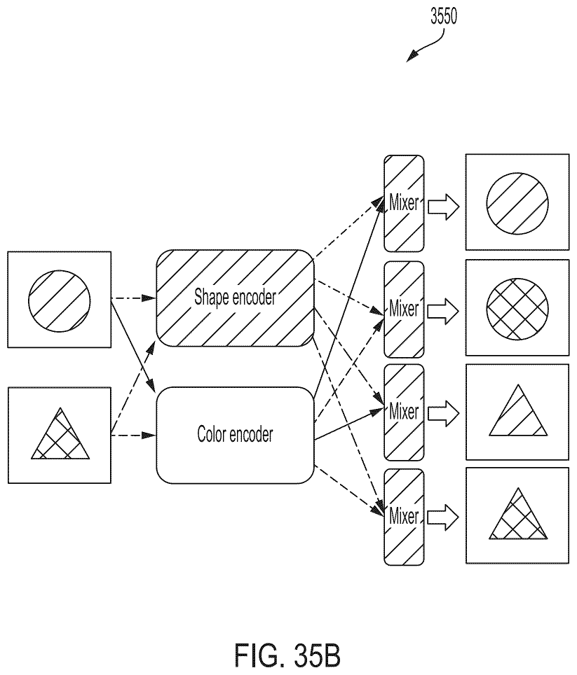

In some embodiments, the trained machine learning model may process the data from the first signal to determine a predicted position of the region of interest relative to the current position (e.g., the position of the region of the brain from which the first signal was detected). In some embodiments, this may include processing the data to detect anatomical landmarks (e.g., ventricles, vasculature, blood vessels, musculature, etc.) and/or motion (e.g., blood flow) in the brain, which may be exploited to determine the predicted position of the region of interest. Based on the predicted position, the machine learning model may determine the direction for forming the second beam and detecting the signal from the region of interest. Machine learning techniques for determining a direction for forming a beam and detecting a signal from the region of interest are described herein including with respect to FIGS. 34 and 35A-B.

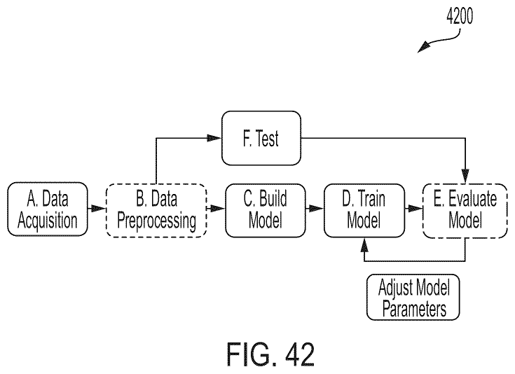

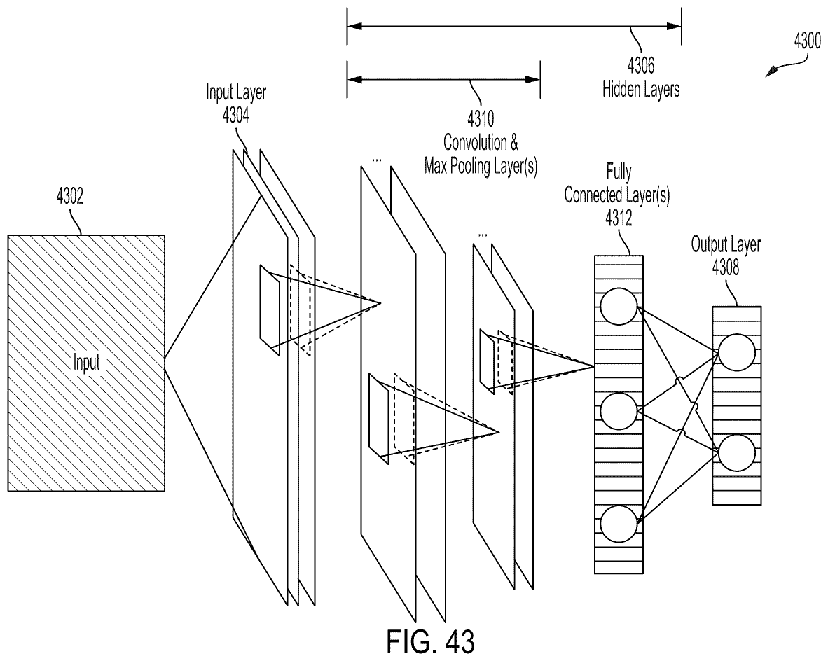

In some embodiments, the machine learning model may be trained on prior signals detected from the brain of one or more persons. The training data may include data generated using machine learning techniques such as Variational Autoencoders (VAE) and Generative Adversarial Networks (GANS) and/or physics based in-silico (e.g., simulation-based) models. An illustrative process for constructing and deploying a machine learning algorithm is described herein including with respect to FIGS. 42-43.

At 3206, based on the output from the machine learning model, the processor, e.g., processor 3004, transmits an instruction to the transducer to detect the signal from the region of interest by forming a beam in the determined direction. In some embodiments, forming a beam (e.g., transmit—and/or receive-beamforming) in the determined direction may include forming a single beam, forming multiple beams, forming beams over a two-dimensional plane, and/or forming beams over a sequence of two-dimensional planes. In some embodiments, the direction of the beam may include the angle of the beam with respect to the face of the transducer.

In some embodiments, detecting the signal from the region of interest of the brain may include autonomously monitoring the region of interest. This may include, for example, monitoring the region of interest using one or more ultrasound sensing modalities, such as pulsatile-mode (P-mode), continuous wave (CW) Doppler, pulse wave (PW)-Doppler, pulse-wave-velocity (PWV), color-flow imaging (CFI), Power Doppler (PD), and/or motion mode (M-mode). In some embodiments, detecting the signal from the region of interest of the brain may include processing the signal to determine the existence and/or the location of a feature in the brain. For example, this may include determining the existence and/or location of an anatomical abnormality and/or anatomical structure in the brain. In some embodiment, detecting the signal from the region of interest of the brain may include processing the signal to segment a structure in the brain, such as, for example, ventricles, blood vessels and/or musculature. In some embodiments, detecting the signal from the region of interest of the brain may include processing the signal to determine one or more brain metrics, such as an intracranial pressure (ICP), cerebral blood flow (CBF), cerebral profusion pressure (CPP), and/or intracranial elastance (ICE). In some embodiments, detecting the signal from the region of interest may correct for beam aberration.

In some embodiments, the region of interest of the brain may include any suitable region(s) of the brain, as aspects of the technology described herein are not limited in this respect. In some embodiments, the region of interest may depend on the intended use of the techniques described herein. For example, for determining a distribution of motion in the brain, a large region of the brain may be defined as the region of interest. As another example, for determining whether there is an embolism in an artery of the brain, a small and precise region may be defined as the region of interest. As yet another example, for measuring blood flow in a blood vessel, two different regions of the brain may be defined as the regions of interest. In some embodiments, a suitable region of any suitable size may be defined as the region of interest, as aspects of the technology are not limited in this respect.

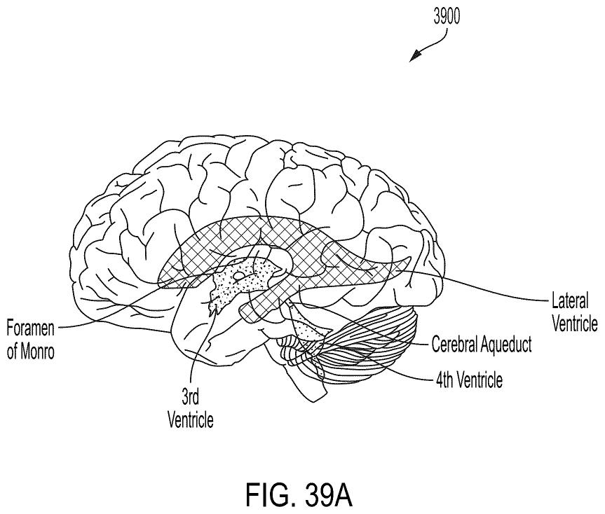

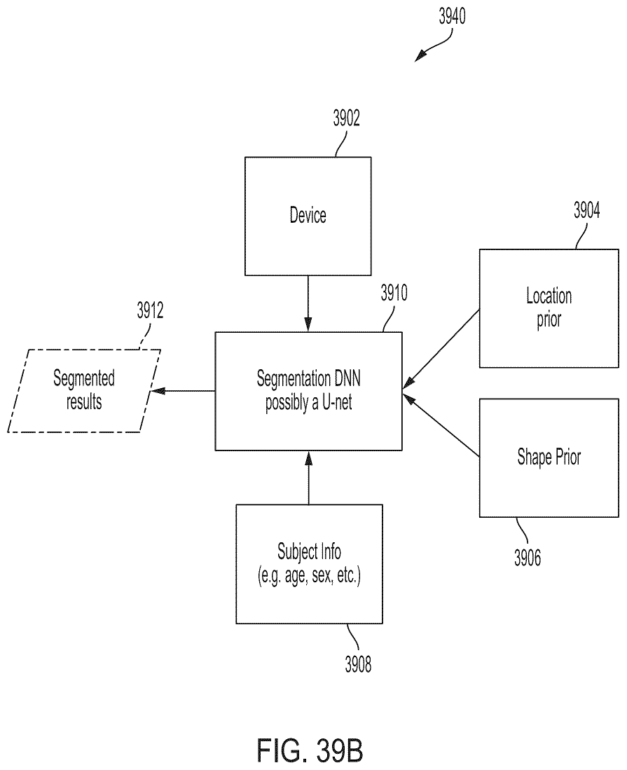

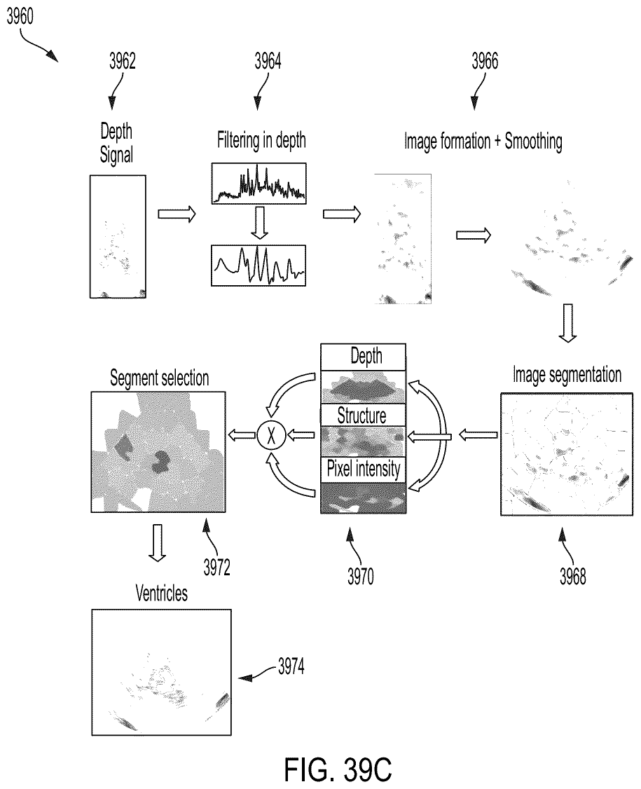

In some embodiments, in identifying a position of a region of interest, the techniques may include detecting, localizing, and/or segmenting anatomical structures in the brain. In addition to aiding in the identification of the region of interest, the results of detection, localization, and segmentation may be useful for informing diagnoses, determining one or more brain metrics, and/or taking measurements of the anatomical structures. Techniques for detecting, localizing, and/or segmenting anatomical structure in the brain are described herein including with respect to FIGS. 32B-32D. Examples for detecting, localizing, and/or segmenting such structures are described herein including with respect to FIGS. 39A-41C.

FIG. 32B shows a flow diagram 810 for a method for detecting, localizing, and/or segmenting a ventricle, according to some embodiments of the technology described herein. In some embodiments, the method may be implemented using a processor, such as processor 3004. Examples for detecting, localizing, and segmenting a ventricle are described herein including with respect to FIGS. 39A-C.

At 3212, the techniques include receiving a signal detected from the brain of a person. In some embodiments, the signal may be received from a transducer (e.g., transducer 3002) configured to detect a signal from a region of interest. For example, the autonomous beam-steering techniques described herein, including with respect to FIG. 8A, may be used to guide a beam towards the region of interest. As other examples, the direction for forming the beam and detecting the signal from the region of interest may be determined based on prior knowledge, output by a machine learning model, and/or identified by a user.

At 3214, data from the detected signal is provided to a machine learning model to obtain an output indicating the existence, location, and/or segmentation of the ventricle. In some embodiments, the data includes image data, such as brightness mode (B-mode) image data.

In some embodiments, the machine learning model may be configured, at 3214a, to cluster the image data to obtain one or more clusters. For example, the image data may be clustered based on pixel intensity, proximity, and/or using any other suitable techniques as embodiments of the technology described herein are not limited in this respect.

At 3214b, the machine learning model is configured to identify, from among the cluster(s), a cluster that represents the ventricle. In some embodiments, the cluster may be identified based on one or more features of the clusters. For example, features used for identifying such a cluster may include a pixel intensity, a depth, and/or a shape associated with the cluster. In some aspects, the features associated with a cluster may be compared to a template of the region of interest. For example, the template may define expected features of the cluster that represents the ventricle such as an estimate pixel intensity, depth, and/or shape. The template may be determined based on data obtained from the brains of one more reference subjects. In some aspects, the techniques may include identifying a cluster that has features that are similar to those of the template.

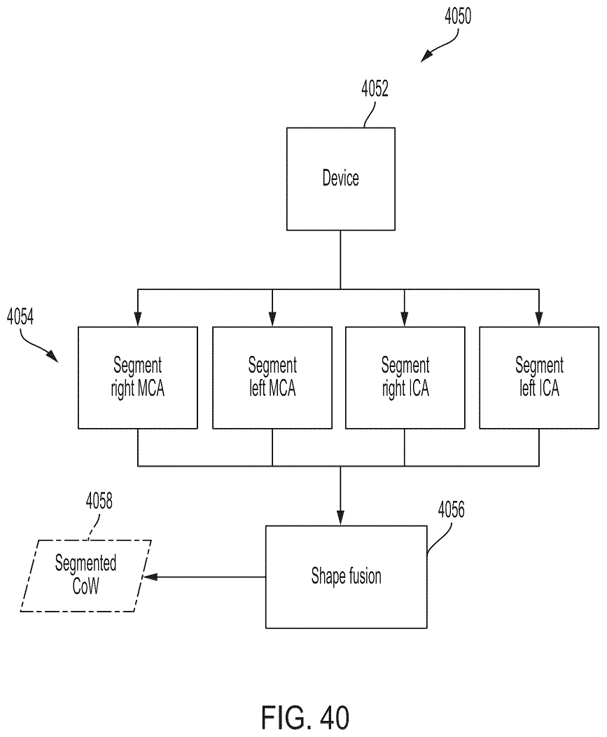

FIG. 32C shows a flow diagram 3220 for detecting, localizing, and/or segmenting the circle of Willis, according to some embodiments of the technology described herein. In some embodiments, the techniques may be implemented using a processor, such as processor 604. Examples for detecting, localizing, and segmenting the circle of Willis are described herein including with respect to FIG. 40

At 3222, the techniques include receiving a first signal detected from the brain of a person. In some embodiments, the first signal may be received from a transducer (e.g., transducer 602) configured to detect a signal from a region of interest. For example, the autonomous beam-steering techniques described herein including with respect to FIG. 32A may be used to guide the beam towards the region of interest. As other examples, the direction for forming the beam and detecting the signal from the region of interest may be determined based on prior knowledge, output by a machine learning model, and/or identified by a user.

At 3224, data from the first signal is provided to a machine learning model to obtain an output indicating the existence, location, and/or segmentation of a first portion of the circle of Willis. In some embodiments, the data includes image data, such as, for example, B-mode image data and/or CFI data. In some embodiments, segmenting the first portion of the circle of Willis may include using the techniques described herein including at least with respect to act 3214 of flow diagram 3210. For example, the machine learning model may be configured to cluster image data and compare features of each cluster to those of a template of the first portion of the circle of Willis.

At 3226, the method includes obtaining a segmentation of a second portion of the circle of Willis. In some aspects, the second portion of the circle of Willis may be segmented according to the techniques described herein including with respect to act 3224. As a non-limiting example, the first portion of the circle of Willis may include the left middle cerebral artery (MCA), while the second portion of the circle of Willis may include the right internal carotid artery (ICA). Additionally or alternatively, a portion of the circle of Willis may include the right MCA, the left ICA, or any other suitable portion of the circle of Willis, as embodiments of the technology described herein are not limited in this respect.

A segmentation of the circle of Willis may be obtained at 3228 based at least in part on the segmentations of the first and second portions of the circle of Willis. For example, obtaining the segmentation of the circle of Willis may include fusing the segmented portions.

In some embodiments, the method 3220 includes segmenting the circle of Willis in portions (e.g., the first portion, the second portion, etc.), rather than in its entirety, due to its size and complexity. However, the techniques described herein are not limited in this respect and may be used to segment the whole structure, as opposed to segmenting separate portions before fusing them together.

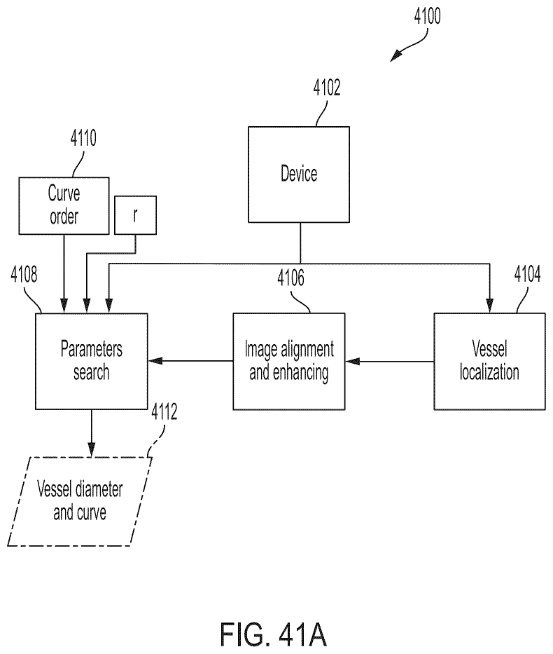

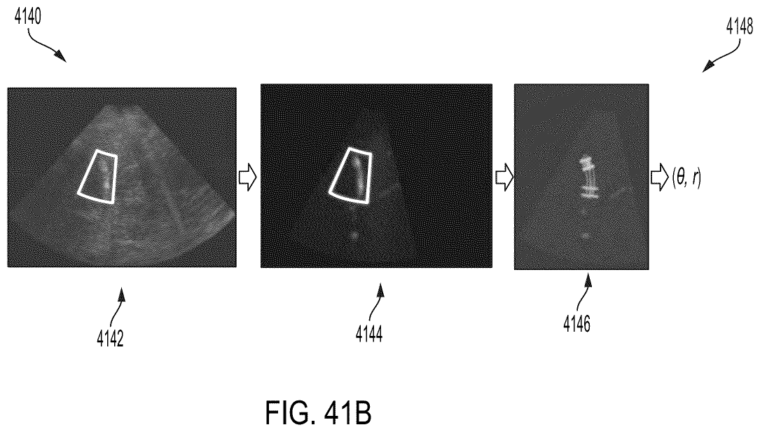

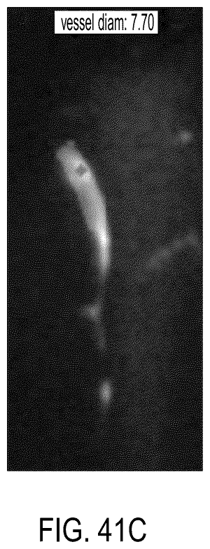

FIG. 832D shows a flow diagram 3230 for a method for localizing a blood vessel, according to some embodiments of the technology described herein. For example, in some embodiments, the techniques may be used to localize portions of the circle of Willis since the circle of Willis includes a network of blood vessels. Examples for detecting and localizing a blood vessel are described herein including with respect to FIGS. 41A-C. In some embodiments, the techniques may be implemented using a processor, such as processor 604.

At 3232, the techniques include receiving a signal detected from the brain of a person. In some embodiments, the signal may be received from a transducer (e.g., transducer 4002) configured to detect a signal from a region of interest. For example, the autonomous beam-steering techniques described herein, including with respect to FIG. 32A, may be used to guide the beam towards the region of interest. As other examples, the direction for forming the beam and detecting the signal from the region of interest may be determined based on prior knowledge, output by a machine learning model, and/or identified by a user.

At 3234, data from the detected signal is provided to a machine learning model to obtain an output indicating the location of the blood vessels. In some embodiments, the date comprises image data, such as brightness mode (B-mode) image data and/or color flow image (CFI) image data.

In some embodiments, the machine learning model is configured, at 3234a, to extract a feature from the provided data. In some embodiments, an extracted feature may include features that are scale and/or rotation invariant. In some embodiments, the features may be extracted utilizing the middle layers of a pre-trained neural network model, examples of which are provided herein.

At 3234b, the extracted features are compared to features extracted from a template of the vessel. In some embodiments, the template may be based on data previously-obtained from the brains of one or more subjects. The results of the comparison may be used to identify the location of the vessel with respect to the image data. In some embodiments, identifying the location based on scale and/or rotation invariant features may help to identify a location with minimal vessel variations. In some embodiments, additional data may be acquired based on the identified location of the vessel (e.g., additional B-mode and/or CFI frames), which may be used for taking subsequent measurements of the vessel and/or blood flow in the vessel.

As described above, features of a region of interest, such as the size, shape, and position, may vary between different people. Thus, it may not be possible to estimate the precise position of the region of interest for each individual based on prior knowledge or training data. For example, the techniques described herein, including with respect to FIGS. 32A and 32B, utilize prior data collected from the brain of subjects in a training population to estimate a position of the region of interest in the subject. However, these techniques may yield only an approximate position of the region of interest. Therefore, the techniques described herein provide for a method for accounting for these subject-dependent variables.

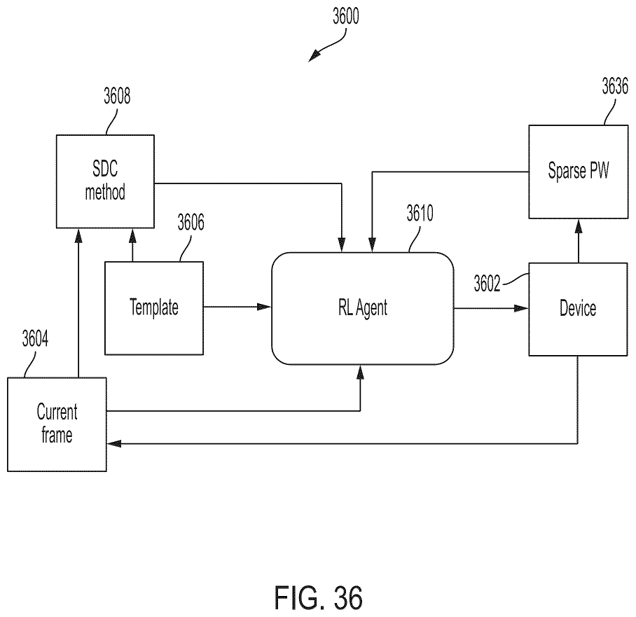

FIG. 32E shows a flow diagram 3240 for a method of locking onto a region of interest, according to some embodiments of the technology described herein. In some embodiments, the method may be implemented using a processor, such as processor 604. Example techniques for locking onto the region of interest are described herein including with respect to FIG. 36.

At 3242, the techniques include receiving a first signal detected from a brain of a person. In some embodiments, the signal may be detected by a transducer (e.g., transducer 3002) forming a beam in a specific direction. For example, the direction may be determined by a user, based on output from a machine learning model (e.g., described herein including with respect to FIGS. 32A and B), based on prior knowledge of the direction for forming the beam, or using any other suitable techniques for determining such a direction, as embodiments of the technology are not limited in this respect.

At 3244, the data from the first signal, as well as an estimate of a position of a region of interest, are provided as input to a machine learning model. For example, the data from the first signal may include B-mode image data, CFI data, PW Doppler data, raw beam data, or any suitable type of data related to the detected signal, as embodiments of the technology are not limited in this respect. In some embodiments, the data from the signal may be indicative of a current region from which the transducer is detecting the signal. The estimate position of the region of interest may be determined based on prior physiological knowledge, prior data collected from the brain of another person or persons, output of a machine learning model, output of techniques described herein including at least with respect to FIGS. 32A-B, data obtained from the detected signal (e.g., the first signal), or determined in any other suitable way as embodiments of the technology are not limited in this respect. In some embodiments, additional information, such as a template of the region of interest may also be provided as input to the machine learning model. For example, a template may provide an estimate position, shape, color, and/or a number of other features estimated for a region of interest.

At 3246, a position of the region of interest is obtained as output from the machine learning model. For example, the machine learning model may include any suitable reinforcement-learning technique for determining the position of the region of interest. In some embodiments, the determined position of the region of interest, output by the machine learning model, may be another estimate position of the region of interest (e.g., not the exact position of the regions of interest).

At 3248, an instruction is transmitted to a transducer to detect a second signal from the region of interest of the brain based on the determined position of the region of interest. In some embodiments, the instruction includes a direction for forming a beam to detect a signal from the region of interest. For example, the direction may be determined based on the output of the machine learning model (e.g., the position of the region of interest) and/or as part of processing data using the machine learning model. In some embodiments, as described above, the determined position of the region of interest may also be an estimate position of the region of interest. Therefore, the instruction may instruct the transducer to detect the second signal from the estimate position of the region of interest determined by the machine learning model, rather than an exact position of the region of interest. In some embodiments, the quality of the second signal may be an improvement over the quality of the first signal. For example, the second signal may have a higher signal-to-noise ratio (SNR) than that of the first signal.

As described above, after locating and/or locking onto a region of interest, it may be desirable to continue to detect signals from the region of interest. However, over time, a signal may no longer be detected from the desired region. For example, due to patient movement, a stick-on probe may become dislodged or slip from its original position. Additionally or alternatively, the beam may gradually shift with respect to the initial direction in which it was formed. Therefore, the techniques described herein provide for techniques for addressing any hardware and/or beam shifts.

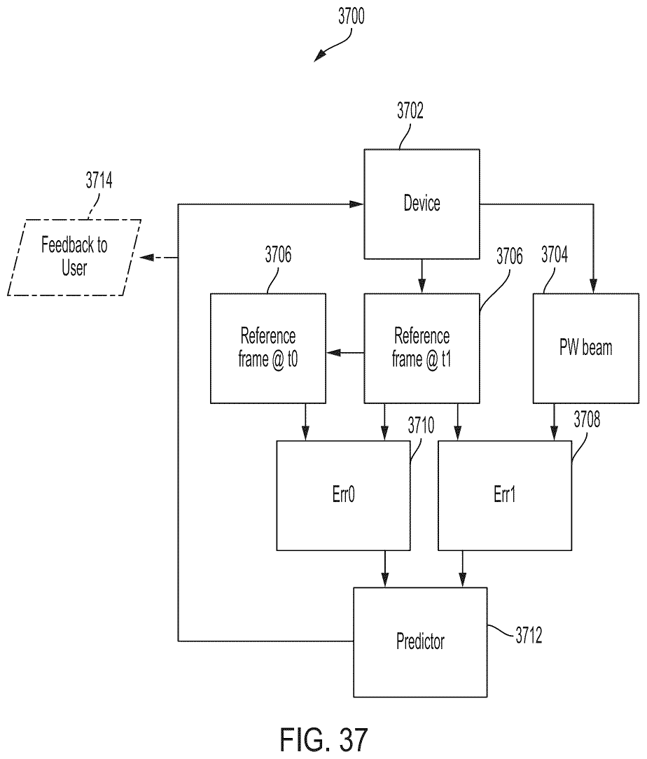

FIG. 32F shows a flow diagram 3250 for a method for estimating a shift due to a shift in hardware, according to some embodiments of the technology described herein. In some embodiments, the method may be implemented using a processor, such as processor 3204. Example techniques are described herein including with respect to FIG. 37.

At 3252, the techniques include receiving a signal detected from a brain of a person. The signal is detected by a transducer (e.g., transducer 3002) forming a beam in a specified direction. For example, the direction may be determined by a user, based on output from a machine learning model (e.g., described herein including with respect to FIGS. 32A, 32B, and 32E), based on prior knowledge of the direction for forming the beam, or using any other suitable techniques for determining such a direction, as embodiments of the technology are not limited in this respect.

At 3254, the techniques include analyzing image data and/or pulse wave (PW) Doppler data associated with the detected signal to estimate a shift associated with the detected signal. In some embodiments, the techniques may include one or more processing steps to process data associated with the signal to obtain B-mode image data and/or PW Doppler data. In some embodiments, analyzing the image data and/or PW Doppler data may include one or more steps. For example, the image data may be analyzed in conjunction with the PW Doppler data to indicate a current position and/or possible angular beam shifts that occurred during signal detection. Additionally or alternatively, a current image frame may be compared to a previously-acquired image frame to estimate a change in position of the region of interest within the image frames over time.

At 3256, the techniques include outputting the estimated shift. For example, the estimated shift may be used as input to a motion prediction and compensation framework, such as a Kalman filter. This may be used to adjust the beam angle to correct for angular shifts, such that the transducer continues to detect signals from a region of interest. Additionally or alternatively, feedback indicative of the estimated shift may be provided through a user interface. For example, based on the feedback, a user may correct for shifts when the hardware does not have the capability.

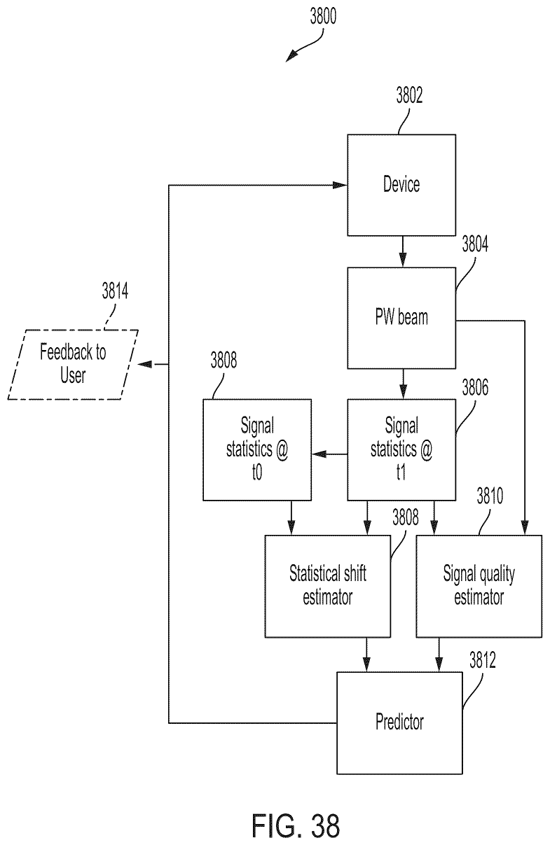

FIG. 32G shows a flow diagram 3260 for a method for estimating a shift associated with the beam, according to some embodiments of the technology described herein. In some embodiments, the method may be implemented using a processor, such as processor 3204. Example techniques are described herein including with respect to FIG. 38.

At 3262, the techniques include receiving a signal detected from a brain of a person. The signal is detected by a transducer forming a beam in a specified direction. For example, the direction may be determined by a user, based on output from a machine learning model (e.g., described herein including with respect to FIGS. 32A, 32B, and 32E), based on prior knowledge of the direction for forming the beam, or using any other suitable techniques for determining such a direction, as embodiments of the technology are not limited in this respect.

At 3264, the techniques include estimating a shift associated with the detected signal. The techniques for estimating such a shift include acts 3264a and 3264b, which may be performed contemporaneously, or in any suitable order.

At act 3264a, statistical features associated with the detected signal are compared with statistical features associated with a previously-detected signal. In some embodiments, the techniques may include estimating a shift based on the comparison of such features. At 3264b, a signal quality of the detected signal is determined. For example, the signal quality may be determined based on the statistical features of the detected signal and/or based on data (e.g., raw beam data) associated with the detected signal. In some embodiments, the output at acts 3264a and 3264b may be considered in conjunction with one another to determine whether an estimated shift is due to a physiological change.

The flow diagram 3260 may proceed to act 3266 when it is determined that the estimated shift is not due to a physiological change. At act 3266, the techniques include providing an output indicative of the estimated shift. For example, the output may be used to determine an updated direction for forming a beam to correct for the shift. Additionally or alternatively, the output may be provided as feedback to a user. The user may be prompted by the feedback to correct for the shift when the hardware does not have this capability.

### Beam-Steering Interrogation Techniques

Some aspects of the technology relate to beam-steering techniques for initially identifying a region of interest. In some embodiments, a beam-steering technique informs the direction for forming the first beam (e.g., the first signal detected at 802 of flow diagram 800) and the number of beams to be formed by the transducer (e.g., a single beam, a two-dimensional plane, a sequence of two-dimensional volumes, a three-dimensional volume, etc.) at one time. In some embodiments, the beam-steering techniques may involve iterating over multiple regions of the brain (e.g., detecting and processing signals from those regions using the machine learning techniques described herein), prior to identifying the region of interest.

FIG. 31 shows example beam-steering techniques. However, it should be appreciated that any suitable beam-steering techniques may be used for identifying a region of interest, as aspects of the technology described herein are not limited in this respect.

Randomized Beam-Steering 3120. In some aspects, the techniques utilize beam-steering at random directions to progressively narrow down the field-of-view to a desired target region, by exploiting a combination of various anatomical landmarks and motion in different compartments. In some embodiments, the machine learning techniques may determine the order in which the sequence is conducted. The system may instantiate a search algorithm by an initial beam (e.g., transmitting and/or receiving an initial beam) that is determined by prior knowledge, such as the relative angle and orientation of the transducer probe with respect to its position on the head. Based on the received beam data at the current and previous states, the system may determine the next best orientation and region for the next scan.

Multi-level (or multi-grid) Beam-Steering 3140. In some aspects, the techniques can utilize a multi-level or multi-grid search space to narrow down the field-of-view to a desired region of interest, starting from a coarse-grained beam-steering (i.e., large spacing/angles between subsequent beams) progressively narrowed down to a finer spacing and angle around the region of interest. The machine learning techniques may determine the degree and area during the grid-refinement process.

Sequential Beam-Steering 3160. In some aspects, the techniques can utilize a sequential beam steering in which case the device steers beams sequentially (in a specific order) over a two-dimensional plane, a sequence of two-dimensional planes positioned or oriented differently in a three-dimensional space, or a three-dimensional volume. The machine learning techniques may determine the order in which the sequence is conducted. With beam-steering merely over a two-dimensional plane or over a three-dimensional volume, the techniques may analyze a full set of beam indices/angles in two dimensions or three dimensions and determine which of the many beams scanned is a fit for the next beam. With a sequence of two-dimensional planar data and/or images (i.e., frame), the techniques may analyze consecutive frames one after another and determine the next two-dimensional plane over which the scan may be conducted.

### Data Acquisition and Processing Pipeline

As described herein, including with respect to 3204 of flow diagram 3200, a processor may receive, from a transducer, data indicative of a signal detected from the brain. In some embodiments, the processor may process the data according to one or more processing techniques. For example, as shown in FIG. 34, the acquired data may be processed according to pipeline 3420 for B-mode (brightness mode) imaging, CFI (color-flow imaging) and PW (pulse-wave) Doppler data. In some embodiments, any combination of processing techniques and/or any additional processing techniques may be used to process the data, as embodiments of the technology described herein are not limited in this respect.

Processing pipeline 3420 shows example processing techniques for B-mode imaging, CFI, and PW Doppler data. For each modality, raw beam data 1004 may undergo time gain compensation (TGC) 3406 to compensate for tissue attenuation. In some embodiments, the data may further undergo filtering 3408 to filter out unwanted signals and/or frequencies. In some embodiments, demodulation 3410 may be performed to remove carrier signals.

After demodulation 3410, processing techniques may vary among the different modalities. As shown, for B-mode imaging, the data may undergo envelope detection 3412 and/or logarithmic compression 3414. In some embodiments, logarithmic compression 1014 may function to adjust the dynamic range of the B-mode images. In some embodiments, the data may then undergo scan conversion 3416 for generating B-mode images. Finally, any suitable techniques 3418 may be used for post-processing the scan converted images.

For CFI, the data may undergo phase estimation 3424, which may be used to inform velocity estimation 3426. In some embodiments, after velocity estimation 3424, the data may undergo scan conversion 3416 to generate CF images. Any suitable techniques 3418 may be used for post-processing the scan converted CF images.

For PW Doppler data, the demodulated data may similarly undergo phase estimation 3424. In some embodiments, a fast Fourier transform (fft) 3428 may be applied to the output of phase estimation 3424, prior to generating sonogram 3430.

In some embodiments, any suitable data (e.g., data acquired from any point in pipeline 3420) may be used as input to machine learning techniques 3444, 3464 for determining the beam-steering strategy 3446, 3466 (e.g., the direction of beamforming for detecting the signal from the region of interest). For example, raw channel or beam data 3442 may be used as input to pipeline 3440, while B-mode and CFI data 3462 may be used as input to pipeline 3460. Other non-limiting examples of input data may include demodulated I/Q data, pre-scan conversion beam data, and scan-converted beam data.

In some embodiments, the machine learning techniques 3444, 3464 may include one or more machine learning techniques that inform the beam-steering strategy 3446, 3466. For example, the machine learning techniques may include techniques for detecting a region of interest, localizing a region of interest, segmenting one or more anatomical structures, locking on a region of interest, correcting for movement due to shifts in hardware, correcting movement due to shifts in the beam, and/or any suitable combination of machine learning techniques. Machine learning techniques are further described herein including with respect to FIGS. 35-43.

### Detection, Localization, and Segmentation of a Region of Interest

In some embodiments, the signals detected during beam-steering, regardless of the technique, may be used to determine a current probing location from which the signals were detected. In some embodiments, the current probing location may be used to assist in detecting, locating, and/or segmenting a region of interest. It can be challenging to determine a probing location based on observation alone, since structural landmarks in B-mode images can be subtle and easy to lose with the naked eye. Further, a full field-of-view three-dimensional space may be relatively large compared to some regions of interest. Accordingly, described herein are AI-based techniques that can be used to analyze beam data to identify the current probing location and/or guide the user and/or hardware towards the region of interest. In some embodiments, the AI-based techniques may be based on prior general structural knowledge provided in the system. For example, the AI-based techniques may exploit structural features (e.g., anatomical structures) and changes in structural features (e.g., motion) to determine a current probing position (e.g., the position of the region of the brain from which a first signal was detected).

In some embodiments, the AI techniques may include using a deep neural network (DNN) framework, trained using self-supervised techniques, to predict the position of a region of interest. Self-supervised learning is a method for training computers to do tasks without labelling data. It is a subset of unsupervised learning where outputs or goals are derived by machines that label, categorize, and analyze information on their own, then draw conclusions based on connections and correlations. In some embodiments, the DNN framework may be trained to predict the relative position of two regions in the same image. For example, the DNN framework may be trained to predict the position of the region of interest with respect to an anatomical structure in a B-mode and/or CF image.

FIG. 35A shows an example diagram of the DNN framework used for estimating the relative positions of two regions in the same image. As shown, a reference patch 3502, at a given position, and a target patch 3504, at an unknown position, are used as input to an encoder 3506. The position estimator 3508 estimates the position of the target patch 3504 with respect to the position of the reference patch 3502.

In some embodiments, the DNN framework may be trained both on two-dimensional and three-dimensional images and/or four-dimensional spatiotemporal data (two- or three-dimensions for space and one-dimension for time). In some embodiments, training the DNN framework may involve obtaining a template for the region of interest. To obtain a template, a disentangling neural network may be trained to extract the region of interest structure and subject-dependent variabilities and combine them to estimate a region of interest shape for a “test” subject. FIG. 35B shows an example algorithm for template extraction. In some embodiments, to achieve a good prior template, the DNN model may be augmented with a classifier that helps the encoder identify an absolute position. This mechanism may improve upon sensitivity to a specific subject, as images from different users may be very different from one another. Additionally, the training may be augmented with a decoder that improves image quality. This may be beneficial in that the embeddings obtained from the encoder network will be rich in information for a more accurate localization.

In some embodiments, given a template of the region of interest and detected signal data (e.g., B-mode image data, CFI data, etc.) from the current probing position, the trained DNN framework may output an indication of the existence of a region of interest, a position of the region of interest with respect to the current probing position, and/or a segmentation of the region of interest. In some embodiments, the output may include a direction for forming a beam for detecting signals from the region of interest. The processor may provide instructions to the transducer to detect a signal from the region of interest by forming a beam in the determined direction.

Due to variability in size, shape, and orientation of structures in the brain (e.g., ventricles, blood vessels, brain tissue, etc.), the AI-based techniques, as described herein above, may be adapted to detect, localize, and/or segment specific structures in the brain. FIGS. 39A-41C describe example techniques for detecting, localizing, and/or segmenting example anatomical structures in the brain, according to some embodiments of the technology described herein.

Ventricle Detection, Localization, and Segmentation. In some embodiments, the techniques described herein may be used to detect, localize, and/or segment ventricles. FIG. 39A shows an example diagram 3900 of ventricles in the brain. Ventricles are critically important to the normal functioning of the central nervous system. The ventricles are four internal cavities that contain cerebrospinal fluid (CSF). CSF flows within and around the brain and spinal cord to help cushion it from injury. This circulating fluid is constantly being absorbed and replenished. There are two ventricles deep within the cerebral hemispheres called the lateral ventricles. They both connect with the third ventricle through a separate opening called the foramen of Monro. The third ventricle connects with the fourth ventricle through a long narrow tube called the aqueduct of Sylvius. From the fourth ventricle, CSF flows into the subarachnoid space where it bathes and cushions the brain. CSF is recycled (or absorbed) by special structures in the superior sagittal sinus called arachnoid villi. A balance is maintained between the amount of CSF that is absorbed and the amount that is produced. A disruption or blockage in the system can cause a build-up of CSF, which can cause enlargement of the ventricles (hydrocephalus) or cause a collection of fluid in the spinal cord (syringomyelia). Additionally, infection (such as meningitis), bleeding or blockage can change the characteristics of the CSF. Brain ventricles' shape can be very useful in diagnosing various conditions such as intraventricular hemorrhage and intracranial hypertension.

FIG. 39B shows a flow diagram 3940 of an example system for ventricle detection, localization, and segmentation. In some embodiments, the detection, localization, and segmentation algorithm may be a classical algorithm and/or a neural network 3910. In some embodiments, the device 3902 provides data, such as B-mode image data as input to the neural network 3910. In some embodiments, additional input, such as location prior 3904, shape prior 3906, and subject information 3908, may be provided as input to the neural network. The location prior 3904 may be indicative of an expected location of the ventricle within the brain. The shape prior 3906 may be indicative of an expected shape of the ventricle. In some embodiments, the location and shape priors 3904, 3906 may be determined based on training data and/or prior knowledge. Example location and shape priors are described herein, including with respect to FIG. 39C. In some embodiments, the subject information 3908 may be used to identify subject dependent variabilities that may depend on age, sex, and/or any other suitable factors. In some embodiments, based on the input, the neural network 3910 may provide segmented results 3912 as output.

FIG. 39C shows a flow diagram illustrating an example segmentation of a ventricle. In some embodiments, data 3962 may be received from the device 3902. In some embodiments, the data may undergo one or more data processing techniques prior to segmentation. After beam-forming and envelope detection, ultrasound data consists of nf×nd×ns tensor, where nf represents number of frames, nd represents number of samples in depth and, ns represents number of sensors. The depth data may contain high frequency information due to inherent speckle noise. However, the brain ventricles are relatively large regions that do not produce high frequency speckle noise. To reduce the inherent speckle noise, a Gaussian averaging may be applied. Also, slow drifts in the data may mislead the algorithm as the edges of ventricles are a band limited feature in depth data. Hence, a dc blocker may be applied to depth data as a high-pass filter. In some embodiments, the dc blocker is defined as:

$$y ( n ) = g \vert x ( n ) - x ( n - 1 ) \vert + Ry ( n - 1 ) \qquad Equation ( 8 )$$

$$g = \frac{1 + R}{2} \qquad Equation ( 9 )$$

For example, as shown in the flow diagram 3960 of FIG. 39C, the depth signal 3962 may be filtered at 3964 to generate filtered beam data. After applying the filter, a scan conversion may be performed to generate a filtered image, as shown at 3966.

In some embodiments, the segmentation techniques may be used to detect plateaus in the filtered image, while maintaining spatial compactness.

$$D = {D}_{lab} + \frac{m}{s} {D}_{xy} \qquad Equation ( 10 )$$

$$where :$$

$$\begin{matrix}s = \sqrt{\frac{N}{K}} , and & Equation ( 11 )\end{matrix}$$

$$\begin{matrix}{D}_{lab}^{i , j} = \sqrt{{( {l}_{i} - {l}_{j} )}^{2} + {( {a}_{i} - {a}_{j} )}^{2} + {( {b}_{i} - {b}_{j} )}^{2}} , and & Equation ( 12 )\end{matrix}$$

$$\begin{matrix}{D}_{x , y}^{i , j} = \sqrt{{( {x}_{i} - {x}_{j} )}^{2} + {( {y}_{i} - {y}_{j} )}^{2}} & Equation ( 13 )\end{matrix}$$

Here, s represents an estimate of super-pixel size which may be computed as the square root ratio of N number of pixels in image and k number of super-pixels. An example of a segmented image is shown at 3968 of flow diagram 3960.

In some embodiments, the target segment (e.g., the ventricle) may include a set of characteristics (e.g., location prior, shape prior, etc.) that may be leveraged during detection. For example, discriminating features may include (a) average pixel intensity, (b) depth, and (c) shape. To incorporate the depth prior (score), a Gaussian kernel may be formed (e.g., σ=nd/5 centered at nd/2, for an example of 5 samples), as ventricles are estimated to be positioned at about the center of the head, with a peak value normalized to one. This one-dimensional vector may then be scan converted to the ultrasound image space. Accordingly, the depth score may be computed as the average of kernel values belonging to that cluster. Flow diagram 3960 illustrates, at 3970, example depth scores (top), calculated according to the techniques described herein. As shown, clusters located near central depths in the image may have a higher score than those clusters located at shallower and/or deeper depths.

In some embodiments, pixels that belong to ventricles may have relatively lower or higher intensity than other pixels. In some embodiments, computing an intensity score for a cluster may include normalizing values to have a mean of zero and a standard deviation of one. The negative average intensity value for each cluster may be computed and transformed according to the nonlinearity in Equation 14, below:

$${score}_{i} ( x ) = \frac{1}{1 + {e}^{\frac{- ( x + 15 )}{0.6}}} \qquad Equation ( 14 )$$

As a result, clusters having a lower intensity may result in a higher score. Flow diagram illustrates, at 3970, example intensity scores (bottom), calculated according to the techniques described herein. As shown, clusters having a lower intensity may result in a higher intensity score.

In some embodiments, ventricles may be also viewed as a particular shape (e.g., shape prior). For example, the ventricles may be viewed as having a similar shape to that of a butterfly in a two-dimensional transcranial ultrasound image. In some embodiments, the shape may be used as a template for scale and invariant shape matching. After smoothing, the template may be used to extract a reference contour for shape scoring. In some embodiments, a contour may be represented as a set of points. For example, the contour may be represented as:

$$c n t {r}_{i} = [ [ {x}_{0}^{i} , {y}_{0}^{i} ] , [ {x}_{1}^{i} , {y}_{1}^{i} ] , … , [ {x}_{{n}_{i}}^{i} , {y}_{{n}_{i}}^{i} ] ] \qquad Equation ( 15 )$$

The center of the contour may be represented as:

$${O}_{i} = [ \frac{1}{{n}_{i}} {\sum }_{j = 0}^{{n}_{i}} {x}_{j}^{i} , \frac{1}{{n}_{i}} {\sum }_{j = 0}^{{n}_{i}} {y}_{j}^{i} ] \qquad Equation ( 16 )$$

In some embodiments, the contour distance curve may be formed by computing the Euclidean distance of every point in cntrᵢ to its center Oᵢ. To mitigate the scale variability, every Dᵢ may be normalized to m_{D}_{ᵢ}=0 and σ_{D}_{ᵢ}=1, ∀i, then a spline may be fit, and all curves may be resampled (e.g., to 200). In addition, to mitigate the rotation variability and to compute a score, the cross-correlation of the template and contours at lags may be estimated. This may be repeated for the first, second and third order derivative of template and other contours and the average of maximum cross-correlation is reported as score. Note that the lag corresponding to maximum correlation may be used to estimate the angle of rotation. The final score for each cluster may then be computed by applying the following nonlinearity:

$${score}_{S} ( x ) = \frac{1}{1 + {e}^{- 10 x}} \qquad Equation ( 17 )$$

Flow diagram 3960 illustrates, at 3970, example shape scores (middle) for each of the clusters. In some embodiments, clusters that have a shape that resemble the shape prior (e.g., the butterfly) may result in a higher shape score.

In some embodiments, a final score may be computed for each cluster by computing the product of the depth, shape, and intensity scores. Example final scores are shown at 3972 of flow diagram 3960. The final selection may be performed by selecting an optimal (e.g., maximum, minimum, etc.) score that satisfies a threshold. For example, selecting a maximum score that exceeds a threshold of 0.75. An example final selection of a cluster is shown at 3974 of flowchart 3960. As shown, the selected cluster corresponds to the highest score from among the scores associated with clusters at 3972.

Circle of Willis Detection and Segmentation. In some embodiments, the techniques described herein may be used to detect, localize, and segment the circle of Willis. The circle of Willis is a collection of arteries at the base of the brain. The circle of Willis provides the blood supply to the brain. It connects two arterial sources together to form an arterial circle, which then supplies oxygenated blood to over 80% of the cerebrum. The structure encircles the middle region of the brain, including the stalk of the pituitary gland and other important structures. The two carotid arteries supply blood to the brain through the neck and lead directly to the circle of Willis. Each carotid artery branches into an internal and external carotid artery. The internal carotid artery then branches into the cerebral arteries. This structure allows all of the blood from the two internal carotid arteries to pass through the circle of Willis. The internal carotid arteries branch off from here into smaller arteries, which deliver much of the brain's blood supply. The structure of the circle of Willis includes, left and right middle cerebral arteries (MCA), left and right internal carotid arteries (ICA), left and right anterior cerebral arteries (ACA), left and right posterior cerebral arteries (PCA), left and right posterior communicating arteries, basilar artery, anterior communicating artery.

As opposed to ventricle detection, localization, and segmentation, template matching methods may not be feasible for the circle of Willis, due to the large template that would be needed.

Template matching methods may not work well for large templates because the speckle noise in the image may mislead the algorithm. Therefore, the circle of Willis may be detected, localized, and/or segmented using the methods described herein including at least with respect to FIGS. 40-41C. A first example method may include separately detecting, localizing, and segmenting different regions of the circle of Willis according to template matching techniques (e.g., such as the techniques described herein, including with respect to FIGS. 39B-C), as shown in flow diagram 4050 of FIG. 40. As shown, data, such as B-mode image data, from the device 4052 may be processed to detect, localize, and segment different regions 4054 of the circle of Willis. For example, the different regions may include the left and right MCA, left and right ICA, left and right PCA, and left and right ACA. In some embodiments, the techniques may include processing the data with a neural network (e.g., neural network 3910) to separately detect, localize, and segment each region. In some embodiments, the segmented regions may then be fused 4056 and provided as output 4058.

Additionally or alternatively, a second example method for detecting, localizing, and segmenting the circle of Willis may include applying techniques described herein for detecting, localizing, and segmenting blood vessels. In some embodiments, as described herein, including at least with respect to FIGS. 41A-C, shape priors and neural networks may be used to extract the circle of Willis from B-mode and CF-images.

Vessel Diameter and Blood Volume Rate. In some embodiments, techniques may be used to determine a vessel diameter and blood volume rate. Traditional matching methods used in computer vision are vulnerable to error in presence of slight shape changes, rotation, and scale. As a result, it may be challenging to determine such blood vessel metrics. Accordingly, described herein are techniques for finding (e.g., detecting and/or localizing) a vessel from B-mode and CF images based on template matching, while addressing these issues.

FIG. 41A shows a flow diagram 4100 of an example system for determining blood vessel diameter and curve. As shown, device 4102 may provide data, such a B-mode image and CF image data, as input to the system. In some embodiments, the techniques utilize pre-trained neural network models and use the output of the middle layers to perform scale and rotation invariant feature extraction. The features may be compared to the features extracted from a template of a vessel to indicate the region of interest location (e.g., vessel localization at 4104 of flow diagram 4100). This may help to create a region of B-mode and color-flow image frames such that vessel location variations are minimal.

As a result, the techniques may obtain a set of frames from the region of interest that are well aligned even in the face of heartbeat, respiration, and probe-induced movements. In some embodiments, image enhancement techniques 4106 may be applied to the aligned region of interest. In some embodiments, averaging the frames may reduce the noise and result in good contrast between the vessel and background. Next, a two-component mixture of Gaussians may be used to cluster foreground and background pixels together. For example, the two components may include pixel value and pixel position. In some embodiments, a polynomial curve may be fit to the foreground and a mask may be created by drawing vertical lines of length r, centered at polynomial. To obtain the best fit, a parameter search 4108 may be conducted over polynomial order and at 4110. This may result in an analytical equation for vessel shape and vessel radius, output at 4112. In some embodiments, vessel shape discovery may also be useful in determining the beam angle to the blood-flow direction that improves PW measurement and accordingly the cerebral blood flow velocity estimates.

FIG. 41B shows an example of determining the diameter and curve of a blood vessel. As shown, the blood vessel is localized at 4142, indicated by the highlighted vessel and border outlining the highlighted vessel. At 4144, alignment and enhancement techniques are applied to the region of interest to reduce the noise and improve the contrast between the vessel and the background. At 4146, a polynomial curve is fit, and a parameter search is conducted to determine output 4148, which may include diameter and curve of the vessel.

As described above, these techniques may be used to detect, localize, and/or segment the circle of Willis. For example, FIG. 41C shows a segmentation of the middle cerebral artery, along with a vessel diameter estimation.

### Active Guidance Mechanism for Locking, Sensing, and Tracking

In some embodiments, the detection and localization techniques described herein may help to determine an approximate position of a region of interest. However, due to variabilities among subjects (e.g., among the brains of subjects), there may be slight inaccuracies associated with the estimated position of the region of interest. In some embodiments, it may be desirable to address these inaccuracies and precisely lock onto the region of interest for an individual. In some embodiments, a fine-tuning mechanism may be deployed in a closed-loop system to precisely lock onto the region of interest. In some embodiments, the techniques may include analyzing one or more signals detected by the transducer to determine an updated direction for forming a beam for precisely detecting signals from the region of interest.

FIG. 36 is a block diagram 3600 showing a system for locking onto a region of interest, according to some embodiments of the technology described herein. In some embodiments, device 3602 may detect signals from the brain. The data may be used to generate one or more B-mode and/or CF image frames 3604. The image frames 3604, along with template 3606, may be used as input to an algorithm for detection and localization 3608 of the region of interest to determine one or more scores (e.g., the scores described with respect to FIG. 15B), a predicted position of the region of interest, and/or a direction for forming the beam for detecting signals from a region of interest. In some embodiments, a reinforcement-learning based algorithm 3610 may map the output of the detection and localization algorithm 3608, the image frame(s) 3604, and the template 3606 to a set of sparse pulse-wave (PW) beams to explore the proximity of the region of interest. In some embodiments, the reinforcement-learning based algorithm 3610 may analyze the quality of the signal 3612, such as the signal-to-noise ratio (SNR), to determine a candidate region of interest. For example, the reflected power of Doppler may be used to estimate the SNR to determine a candidate region of interest. The processor may provide the output of the reinforcement-learning based algorithm 3610 to the transducer, instructing the transducer to detect the signal from the refined position (e.g., the candidate region of interest). In some embodiments, the process is repeated (e.g., using beam data acquired from the candidate region of the brain) until the algorithm converges and/or a time threshold is reached. In some embodiments, detecting the signal from the refined position may help to lock on the region of interest and improve the SNR (e.g., increase the SNR).

Active target tracking. During continuous recording, it can be challenging to keep the hardware on target, despite the closed-loop mechanisms for locking onto the region of interest. Shifts and/or drifts in the hardware (e.g., the transducer) may occur, even though the hardware may be designed to lock in place (e.g., on the region of interest) and keep a sturdy hold in position. In some embodiments, a live tracking system may be used to address hardware shifts and/or drifts based on a Kalman filter.

FIG. 37 is a block diagram 3700 showing a system for determining and/or measuring drifts associated with hardware. In some embodiments, the techniques include acquiring PW beams 3704, in PW Doppler mode, from one or a few angles in high frequency using device 3702. The techniques may further include recording a B-mode image 3706, 3710 at a relatively low frequency (e.g., once every second and/or every few seconds) using device 3702, during the PW recordings. In some embodiments, using a speckle correlation analysis, a most recently recorded B-mode image frame 3706 may be used as a reference to indicate the location and possible angular shifts in the current PW beam 3704. An estimated undesirable shift 3708 is provided as input to a motion prediction and compensation framework (e.g., a Kalman filter) 3714, which determines an updated direction for forming a beam (e.g., a PW beam) to keep the beam on target (e.g., on the region of interest). In some embodiments, the updated direction may be provided as feedback 3716 to the transducer for course-correction. Additionally or alternatively, to ensure that the reference B-mode image frame 3706 still captures the correct slice and to determine the template shift 3712 over time, the B-mode image frame 3706 is compared to a previously-acquired B-mode image frame 3710. For example, the B-mode image frames 3706, 3710 may be compared using an order-one Markovian model. In some embodiments, the output of the comparison may be provided as feedback 3716 to the transducer to adjust for B-mode frame shifts (e.g., update the direction for forming the beam). Additionally or alternatively, the output of the comparison may be provided as feedback 3716 to a user if the device focus point is moving out of the plane of the target region and there is no hardware capability for correcting for the shift.

Course Correcting Component. Though the techniques may lock the system on target, the beam may gradually shift, or the contact quality may change during the course of measurement. To address this, in some embodiments, the techniques may monitor the signal quality and, upon observing a statistical shift that does not translate to physiological changes, it may (a) perform a limited search around the region of interest to fix the limited shift without interrupting the measurements, and/or (b) upon observing substantial dislocations, engages the reinforcement-learning algorithm for realigning and/or alerting the user of contact issues if the search was unsuccessful.

FIG. 38 is a block diagram 3800 showing a system for determining and/or measuring shifts in the beam. As shown, the techniques include acquiring a beam in PW Doppler mode at a time 11, using device 3802. In some embodiments, the system extracts the statistical features 3806 of the beam acquired at time 11. The statistical features 3806, along with the raw beam data 3804 are used as input to a signal quality estimator 3812 to determine if the data satisfies certain conditions (e.g., the signal quality is satisfactory). Additionally or alternatively, the statistical features 3806 of the beam 3804 acquired at time 11 are compared to statistical features extracted from a previously-acquired beam 3808 to estimate statistical shifts 3810. In some embodiments, the statistical shift estimator 3810 may include a Siamese DNN, which may look for a substantial shift, as well as slow drifts, in statistics of the signal and classify the nature of the shifts and/or drifts. In some embodiments, the outputs of the statistical shift estimator 3810 and the signal quality estimator 3812 may be used to determine a course of action if a shift occurs (e.g., using predictor 3814). For example, the output may be provided to a DNN-based Kalman filter for tracking three-dimensional motion using the signal quality. In some embodiments, the output of the predictor 3814 may be provided as feedback 3816 to the transducer for forming a beam in an updated direction (e.g., for correcting for the shift). Additionally or alternatively, feedback 3816 may be provided to a user for adjusting the hardware and/or providing an indication of the shift.

### Autonomous Sensing

In some embodiments, once locked onto a region of interest, the system (e.g., system 600) may continuously and/or autonomously monitor the region of interest using any suitable ultrasound modality. For example, ultrasound modalities may include continuous wave (CW) Doppler, pulse wave (PW) Doppler, pulsatile-mode (P-mode), pulse-wave-velocity (PWV), color flow imaging (CFI), power Doppler (PD), motion mode (M-mode), and/or any other suitable ultrasound modality, as aspects of the technology described herein are not limited in that respect.

Additionally or alternatively, once locked onto a region of interest, the system (e.g., system 600) may sense and/or monitor brain metrics from the region of interest. For example, brain metrics may include intracranial pressure (ICP), cerebral blood flow (CBF), cerebral perfusion pressure (CPP), intracranial elastance (ICE), and/or any suitable brain metric, as aspects of the technology described herein are not limited in this respect.

### Artificial Intelligence (AI) in Smart Beam Steering

As described herein, AI can be used on various levels such as in guiding beam steering and beam forming, detection, localization, and segmentation of different landmarks, tissue types, vasculature and physiological abnormalities, detection and localization of blood flow and motion, autonomous segmentation of different tissue types and vasculature, autonomous ultrasound sensing modalities, and/or sensing and monitoring brain metrics, such as intracranial pressure, intracranial elastance, cerebral blood flow, and/or cerebral profusion.

In some embodiments, beam-steering may employ one or more machine learning algorithms in the form of a classification or regression algorithm, which may include one or more sub-components such as convolutional neural networks, recurrent neural networks such as LSTMs and GRUs, linear SVMs, radial basis function SVMs, logistic regression, and various techniques from unsupervised learning such as variational autoencoders (VAE), generative adversarial networks (GANs) which are used to extract relevant features from the raw input data.

Exemplary steps 4200 often undertaken to construct and deploy the algorithms described herein are shown in FIG. 42, including data acquisition, data preprocessing, building a model, training the model, evaluating the model, testing, and adjusting model parameters.

FIG. 43 shows a convolutional neural network 4300 that may be employed by the AEG device. The statistical or machine learning model described herein may include the convolutional neural network 4300, and additionally or alternatively another type of network, suitable for predicting frequency, amplitude, acoustic beam profile, and other requirements, such as expected temperature elevation and/or radiation force, etc. As shown, the convolutional neural network comprises an input layer 4304 configured to receive information about the input 4302 (e.g., a tensor), an output layer 4308 configured to provide the output (e.g., classifications in an n-dimensional representation space), and one or more hidden layers 4306 connected between the input layer 4304 and the output layer 4308. The hidden layer(s) 4306 include convolution and pooling layers 4310 and fully connected layers 4312.

The input layer 4304 may be followed by one or more convolution and pooling layers 4310. A convolutional layer may comprise a set of filters that are spatially smaller (e.g., have a smaller width and/or height) than the input to the convolutional layer (e.g., the input 4302). Each of the filters may be convolved with the input to the convolutional layer to produce an activation map (e.g., a 2-dimensional activation map) indicative of the responses of that filter at every spatial position. The convolutional layer may be followed by a pooling layer that down-samples the output of a convolutional layer to reduce its dimensions. The pooling layer may use any of a variety of pooling techniques such as max pooling and/or global average pooling. In some embodiments, the down-sampling may be performed by the convolution layer itself (e.g., without a pooling layer) using striding.

The convolution and pooling layers 4310 may be followed by fully connected layers 4312. The fully connected layers 4312 may comprise one or more layers each with one or more neurons that receives an input from a previous layer (e.g., a convolutional or pooling layer) and provides an output to a subsequent layer (e.g., the output layer 4308). The fully connected layers 4312 may be described as “dense” because each of the neurons in a given layer may receive an input from each neuron in a previous layer and provide an output to each neuron in a subsequent layer. The fully connected layers 4312 may be followed by an output layer 4308 that provides the output of the convolutional neural network. The output may be, for example, an indication of which class, from a set of classes, the input 4302 (or any portion of the input 4302) belongs to. The convolutional neural network may be trained using a stochastic gradient descent type algorithm or another suitable algorithm. The convolutional neural network may continue to be trained until the accuracy on a validation set (e.g., a held-out portion from the training data) saturates or using any other suitable criterion or criteria.

It should be appreciated that the convolutional neural network shown in FIG. 43 is only one example implementation and that other implementations may be employed. For example, one or more layers may be added to or removed from the convolutional neural network shown in FIG. 43. Additional example layers that may be added to the convolutional neural network include: a pad layer, a concatenate layer, and an upscale layer. An upscale layer may be configured to up-sample the input to the layer. An ReLU layer may be configured to apply a rectifier (sometimes referred to as a ramp function) as a transfer function to the input. A pad layer may be configured to change the size of the input to the layer by padding one or more dimensions of the input. A concatenate layer may be configured to combine multiple inputs (e.g., combine inputs from multiple layers) into a single output. As another example, in some embodiments, one or more convolutional, transpose convolutional, pooling, un-pooling layers, and/or batch normalization may be included in the convolutional neural network. As yet another example, the architecture may include one or more layers to perform a nonlinear transformation between pairs of adjacent layers. The non-linear transformation may be a rectified linear unit (ReLU) transformation, a sigmoid, and/or any other suitable type of non-linear transformation, as aspects of the technology described herein are not limited in this respect.

Convolutional neural networks may be employed to perform any of a variety of functions described herein. It should be appreciated that more than one convolutional neural network may be employed to make predictions in some embodiments. Any suitable optimization technique may be used for estimating neural network parameters from training data. For example, one or more of the following optimization techniques may be used: stochastic gradient descent (SGD), mini-batch gradient descent, momentum SGD, Nesterov accelerated gradient, Adagrad, Adadelta, RMSprop, Adaptive Moment Estimation (Adam), AdaMax, Nesterov-accelerated Adaptive Moment Estimation (Nadam), AMSGrad.

### Pulsatility-Mode Mode Sensing Techniques

Brain tissue motion and pulsatility may be measured using various techniques, including, but not limited to, standard continuous wave doppler, or pulsed wave doppler, color doppler, or power doppler techniques, where the doppler effect due to the motion of subwavelength scatterers in brain tissue or blood is captured by measuring the shift in the carrier frequency or phase of the received waveforms. Alternatively, the numerous subwavelength scatterers present in biological tissue generate a seemingly random interferential pattern commonly referred to as “ultrasonic speckle”. The motion of the subwavelength scatterers leads to changes in the speckle pattern that can be tracked in time. Thus, by tracking speckles as a function of time, one can extract the motion of brain tissue or blood cells. Various filtering techniques may be applied to extract the motion at the frequency range of interest. Aspects of these filtering techniques for measuring brain tissue pulsatility will now be discussed.

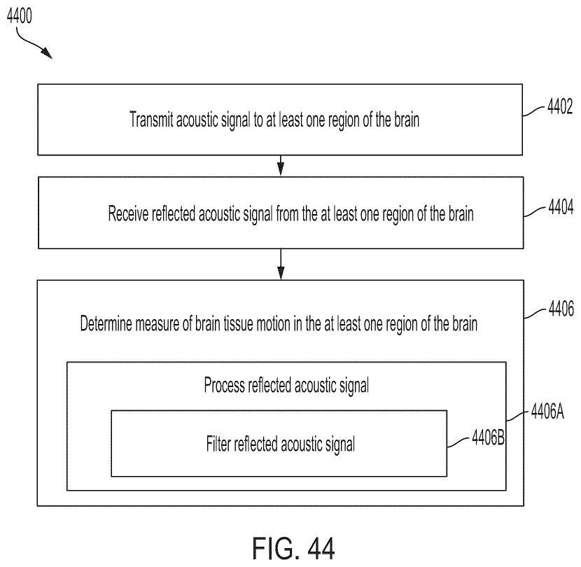

FIG. 44 shows an illustrative process 4400 for determining a measure of brain tissue motion in a brain, according to some embodiments. The process 4400 may begin at act 4402, where an acoustic signal is transmitted to at least one region of the brain. For example, as described herein, the acoustic signal may be transmitted by at least one ultrasonic transducer. In some embodiments, the acoustic signal may comprise one or more acoustic signals. In some embodiments, the acoustic signal(s) may form an acoustic beam. In some embodiments, the acoustic beam may be steered to the at least one region of the brain by controlling delays of the ultrasonic transducers.

As described herein, in some embodiments, the at least one ultrasonic transducer may comprise one or more ultrasonic transducers. In some embodiments, the ultrasonic transducer(s) may be arranged in an array.

At act 4404, a reflected acoustic signal may be received from the at least one region of the brain. For example, the received acoustic signal may comprise a reflection of the acoustic signal transmitted at act 4402. In some embodiments, the received acoustic signal may comprise at least a portion of the acoustic signal transmitted at act 4402 that has either been reflected or refracted by the at least one region of the brain.

The acoustic signal may be received by at least one ultrasonic transducer. In some embodiments, the at least one ultrasonic transducer that receives the signal comprises one or more of the at least one ultrasonic transducer that transmitted the acoustic signal at act 4402. In some embodiments, the at least one ultrasonic transducer that receives the signal is different than the at least one ultrasonic transducer that transmitted the acoustic signal at act 4402.

At act 4406, a measure of brain tissue motion in the at least one region of the brain is determined. For example, the measure of brain tissue motion may be determined based on the acoustic signal received at act 4404. For example, as shown in FIG. 44, determining the measure of brain tissue motion in the at least one region of the brain may comprise processing the reflected acoustic signal at act 4406A. Processing the reflected acoustic signal may comprise applying one or more techniques to filter the reflected acoustic signal at act 4406B. Examples of the one or more techniques that may be applied to filter the reflected acoustic signal and determine a measure of brain tissue motion in the at least one region of the brain will now be described.

a. Spatiotemporal Filtering

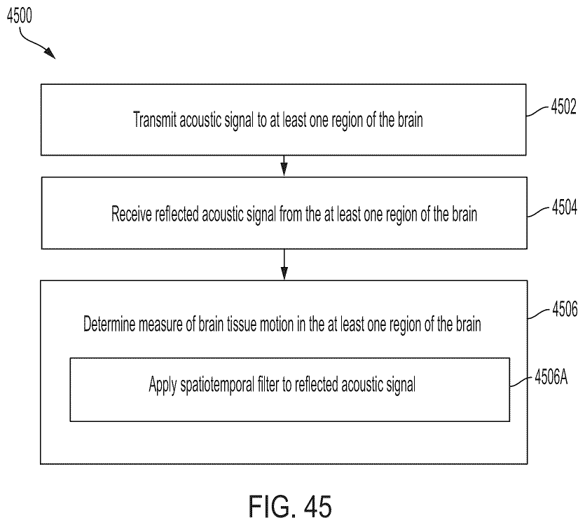

According to one embodiment, a spatiotemporal filtering technique may be performed to extract brain tissue motion. FIG. 45 shows an illustrative process 4500 for determining a measure of brain tissue motion in a brain via spatiotemporal filtering, according to some embodiments.

The process 4500 may begin at act 4502. Acts 4502-4506 may be performed in the same manner as acts 4402-4406 of process 4400.

At act 4506A, the measure of brain tissue motion in the at least one region of the brain may be determined at least in part by applying a spatiotemporal filter to the reflected acoustic signal.

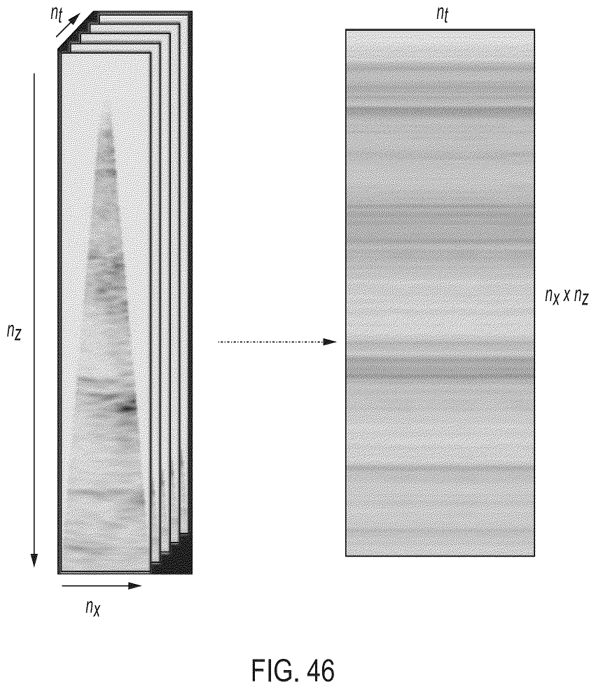

For example, the brain may be imaged using an ultrasound scanner and the brain tissue motion is extracted from a series of images using advanced spatiotemporal filtering techniques such as, but not limited to, Singular Value Decomposition (SVD), a matrix decomposition technique related to Principle Component Analysis (PCA). In this approach, an ultrasonic imaging device produces one or more B-mode (intensity) images of the brain. Several images of the brain may be collected at a certain frame rate, totaling a given number of seconds worth of images. The dataset thusly acquired can be seen as a three-dimensional array with spatial dimension x and z (azimuth and depth, respectively) and a temporal dimension t (time) containing nₓ×n_{z}×n values. S(x, z, t) denotes the dataset. FIG. 46 shows an illustrative example of unwrapping a three-dimensional image stack into a two-dimensional matrix, according to some embodiments. As seen in FIG. 46, the three-dimensional array containing the data can be rearranged into a two-dimensional array of size (nₓ×n_{z})×nₜ by unwrapping the z dimension column-wise.

S denotes this two-dimensional array. This two-dimensional matrix is suitable for SVD filtering. The first dimension of S corresponds to space whereas the second dimension is time. Performing the SVD decomposition of S amounts to finding the matrices U, Σ, and V such that:

$$S = U Σ {V}^{T}$$

where U is an (nₓ·n_{z}×nₓ·n_{z}) orthonormal matrix, Vis an (nₜ×nₜ) orthonormal matrix, Σ is an (nₓ·n_{z}×nₜ) diagonal, non-square matrix containing the singular values σᵢ of S:

$$Σ = ( {σ}_{1} 0 … 00 ⋱ ⋱ ⋮ ⋮ ⋱ ⋱ 0 ⋮ ⋱ {σ}_{n t} ⋮ 0 ⋮ ⋮ 0 … … 0 )$$

V^{T} is the conjugate transpose of V. From this, it can be seen that the columns of U are the spatial vectors of S and the columns of V are the temporal singular vectors of S.

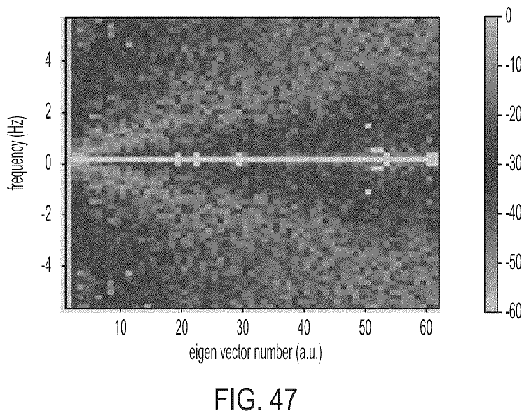

FIG. 47 shows an illustration of the power spectra of temporal singular vectors in decreasing singular value order, according to some embodiments. FIG. 47 shows the spectra of the column vectors of V ordered by decreasing singular value. It can be seen that larger singular values are associated with temporal vectors V containing lower temporal frequencies, whereas smaller singular values progressively incorporate higher and higher temporal frequency content. Some of this higher frequency content is noise.

Thus, this decomposition captures the spatiotemporal variations of S in a separable form. Using this decomposition, S may be expressed as a weighted sum of the outer product of the columns of U with the columns of V:

$$S = {\sum }_{i} {σ}_{i} \cdot {U}_{i} \times {V}_{i}$$

The column Vᵢ describes the temporal variation associated with the corresponding spatial column Uᵢ. The spatial column vector Uᵢ of size (nₓ×n_{z}, 1), can be treated as a sub-image by Iᵢ wrapping it column-wise. It can be seen that the temporal column vector Vᵢ thus modulates the intensity of the pixels in Iᵢ in time. In other words, the intensity of all the pixels in the sub-image Iᵢ have the same temporal behavior characterized by Vᵢ. As a result, spatiotemporal decomposition for each pixel of S has been achieved.

With the number of non-zero σᵢ corresponding to the rank of S, we have:

$$s ( x , z , t ) = {\sum }_{i = 1}^{rank ( S )} {σ}_{i} {I}_{i} ( x , z ) {V}_{i} ( t )$$

The σᵢ are in decreasing order. The first and largest singular values σᵢ can be expected to be associated with static tissue since it represents the structure with the highest spatiotemporal coherence. The spatiotemporal signal associate with brain tissue emotion can be found in the other singular values, excluding the high frequency noise. Therefore, a spatiotemporal filter capable of isolating brain tissue motion simply by setting the first few singular values σᵢ of S to zero can be achieved. In other words, the filtered matrix S_{f} can be built such that:

$${S}_{f} = U {Σ}_{f} {V}^{T}$$

The matrix Σ_{f} can be tuned to reject certain spatiotemporal signals and preserve others. For example, if one wants to rid the dataset from still tissue, one can set Σ_{f} to be:

$${Σ}_{f} = ( 00 … 00 {σ}_{2} ⋱ ⋮ ⋮ ⋱ ⋱ 0 ⋮ ⋱ {σ}_{{n}_{t}} ⋮ 0 ⋮ ⋮ 0 … … 0 )$$

Further, the σᵢ values may be amplified or attenuated based on the desired results. For example, in the illustrated embodiment, the σᵢ values are used to preserve brain tissue motion and reject any other spatiotemporal signal as clutter.

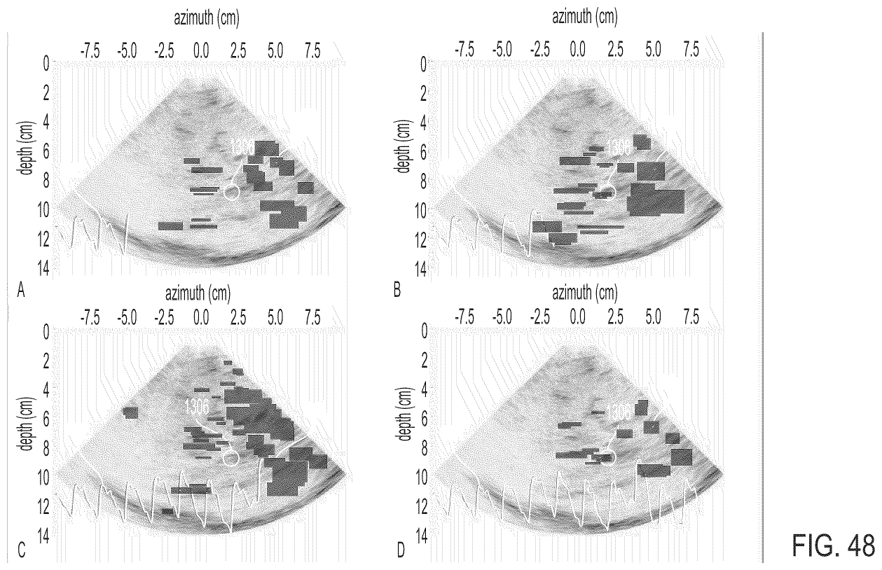

FIG. 48 shows examples of singular value decomposition filtering on a sequence of B-mode images of a patient's brain, according to some embodiments. FIG. 48 shows a dataset processed using the SVD filtering technique described herein. The grayscale images correspond to several ultrasound B-mode frames of a patient's brain. The overlays 4802 and inside circle 4806 shows where a beating motion is occurring in the brain. Lines 4804 indicates a decreasing pixel intensity whereas a coloration inside circle 4804 indicates increasing pixel intensity with time. The trace plotted at the bottom of each frame shows the variations of the pixel intensity in the circle 4802. Isolating the parts of the image where the brain tissue is beating with the patient's heartbeat may facilitate assessment of the stiffness of the surrounding tissue.

b. Signal Decomposition

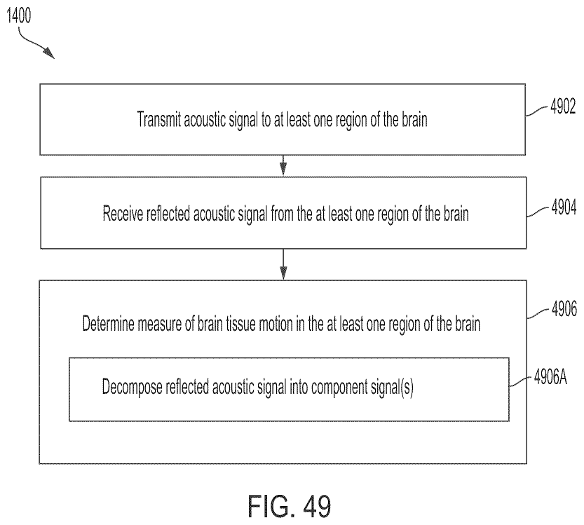

According to one embodiment, a signal decomposition technique may be performed to extract brain tissue motion. FIG. 49 shows an illustrative process for determining a measure of brain tissue motion in a brain via signal decomposition, according to some embodiments.

The process 4900 may begin at act 4902. Acts 4902-4906 may be performed in the same manner as acts 4402-4406 of process 4400.

At act 4906A, the measure of brain tissue motion in the at least one region of the brain may be determined at least in part by decomposing the reflected acoustic signal into one or more component signals.

The goal of signal decomposition is extraction and separation of signal components from composite signals, which should preferably be related to semantic units. Examples of such signal components in composite signals are distinct objects in images or video, video shots, melody sequences in music, spoken words or sentences in speech signals. The criteria selected for separating the signals enables one to decompose a superimposed signal into components that are separable considering different aspects. For example, one example signal decomposition technique is linear discriminant analysis (LDA). LDA solves to find a subspace with an orthogonal basis in which the signals are linearly separable and the theoretical basis for many statistical signal processing algorithms holds. Other techniques may be used, such as the techniques described herein including Kernal Principal Component Analysis and Blind Source Separation, which may carry advantages over LDA. For instance, LDA assumes a linear relationship between components and measured signal, but most of the time this is not correct.

i. Kernal Principal Component Analysis (KPCA)

In some embodiments, kernel principal component analysis (KPCA) may be used to extract brain tissue motion from a set of ultrasound images. In the field of multivariate statistics, KPCA is an extension of principal component analysis (PCA) using techniques of kernel methods. Using a kernel, the originally linear operations of PCA are performed in a reproducing kernel Hilbert space.

Kernel methods owe their name to the use of kernel functions, which enable them to operate in a high-dimensional, implicit feature space without ever computing the coordinates of the data in that space, but rather by computing the inner products between the images of all pairs of data in the feature space. This approach is called the “kernel trick”.

Kernel functions can be non-linear but restricted by a set of constraints. For example, to extract brain tissue motion, each pixel time series may be assumed to be a measured superimposed signal in time. It may also be assumed that the components leading to these pixel time series are common among all. Accordingly, the reproducible kernel Hilbert space orthogonal basis may be solved for.

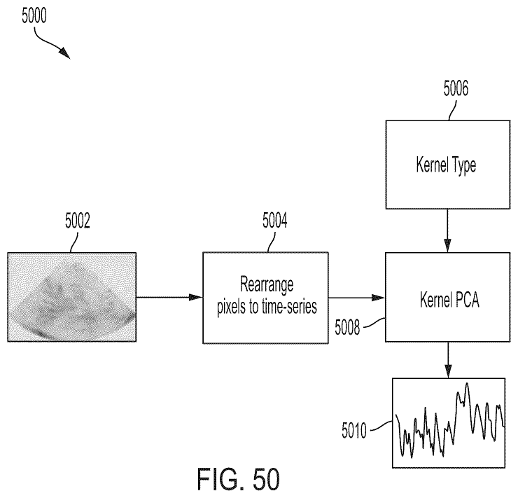

FIG. 50 shows an example flow diagram 5000 of kernel principal component analysis, according to some embodiments. The process illustrated in flow diagram 5000 may begin at act 5002 where one or more images are obtained. For example, the image(s) may comprise a one or more B-mode ultrasound images.

At act 5004, pixels in the image(s) obtained at act 5002 may be rearranged into a time-series. At act 5006, a kernel type may be selected for use in the signal decomposition technique. Any suitable kernel technique may be selected. For example, in some embodiments, a cosine kernel may be implemented.

At act 5008, principal component analysis may be performed using the kernel type selected at act 5006. The goal of act 5008 is to decompose the signals obtained at act 5002 to identify at least one signal representative of brain tissue motion. Plot 5010 shows an example of a signal extracted from the image(s) using KPCA.

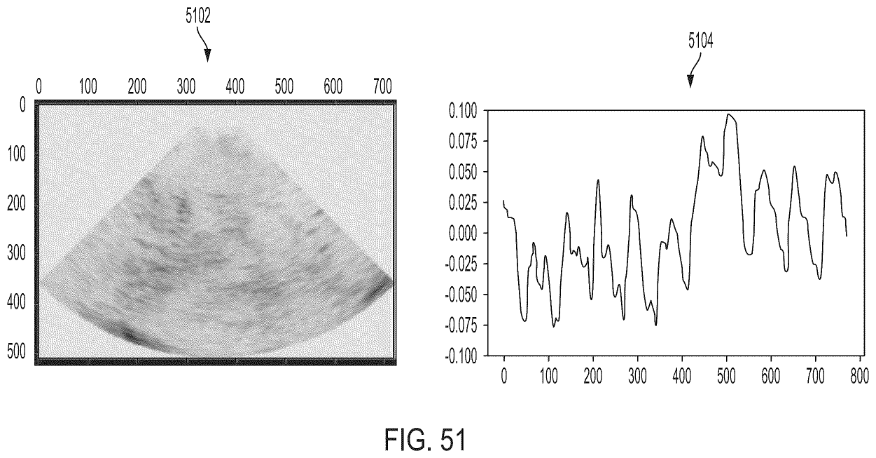

FIG. 51 shows example images of extracted components from kernel principal component analysis, according to some embodiments. In the illustrated example, a ‘cosine’ kernel and a time series of brightness mode images 5102 in a plane that contains brain ventricles was used. The extracted signal is presented in plot 5104.

ii. Blind Source Separation (BSS)

In some embodiments, Blind Source Separation (BSS) may be used to extract brain tissue motion from a set of ultrasound images. BSS refers to a problem where both the sources and the mixing methodology are unknown, only mixture signals are available for further separation processing. In several situations it is desirable to recover all individual sources from the mixed signal, or at least to segregate a particular source. There are various methods with different assumptions to identify underlying signal sources and/or mixing forward models e.g. common spatial patterns, stationary subspace analysis, dependent component analysis, independent component analysis (ICA) etc.

For example, in some embodiments, independent component analysis (ICA) may be implemented for separating signal sources from brightness image time-series. In signal processing, ICA is a computational method for separating a multivariate signal into additive subcomponents. This may be performed by assuming that the subcomponents are non-Gaussian signals and that they are statistically independent from each other.

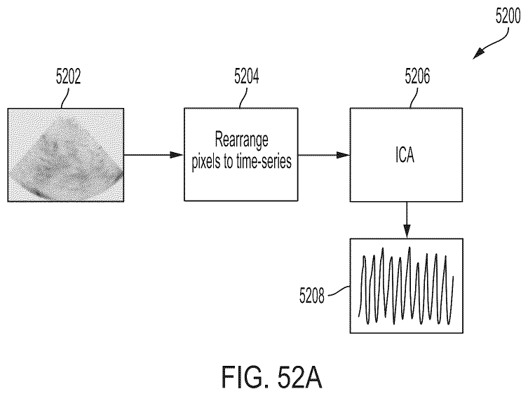

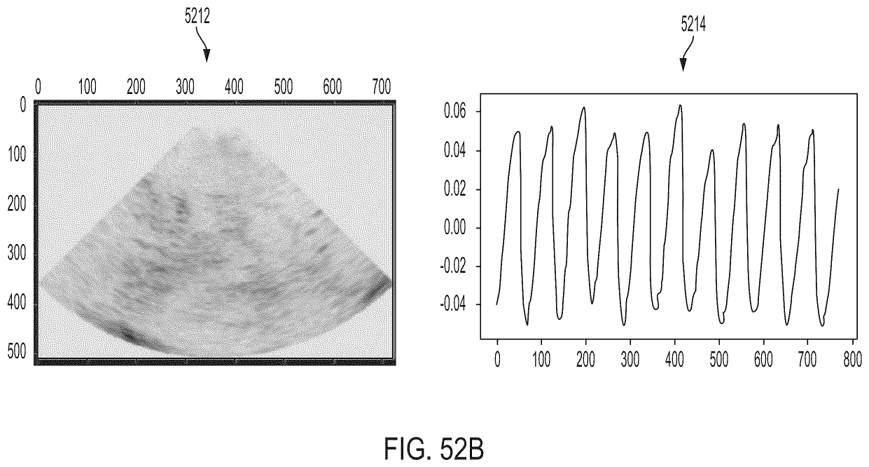

FIG. 52A shows an example flow diagram 5200 of independent component analysis, according to some embodiments. The process illustrated in flow diagram 5200 may begin at act 5202 where one or more images are obtained. For example, the image(s) may comprise a one or more B-mode ultrasound images.

At act 5204, pixels in the image(s) obtained at act 5202 may be rearranged into a time-series. At act 5206, independent component analysis may be performed. The goal of act 5206 is to decompose the signals obtained at act 5202 to identify at least one signal representative of brain tissue motion. Plot 5208 shows an example of a signal extracted from the image(s) using ICA.

FIG. 52B shows example images of extracted components from independent component analysis, according to some embodiments. In particular, image 5212 shows an example of a B-mode image of brain tissue obtained from a region of the brain. Plot 5214 shows an example of an extracted signal obtained via signal decomposition according to process 5200. The extracted signal may be representative of brain tissue motion in the region of the brain.

ICA is a special case of blind source separation. An example application of ICA is the “cocktail party problem” of listening in on one person's speech in a noisy room. To perform this analysis one can propose a linear forward model as the mixing function or propose other non-linear methods.

In some embodiments, one or more blind source separation techniques may be used in addition or alternative to the techniques described herein. For example, in some embodiments, non-linear ICA may be used. For example, the relationship between different beating signal waveshapes, including intracranial pressure morphology, could be nonlinearly encoded in speckle's temporal statistics.

c. Tissue Tracking

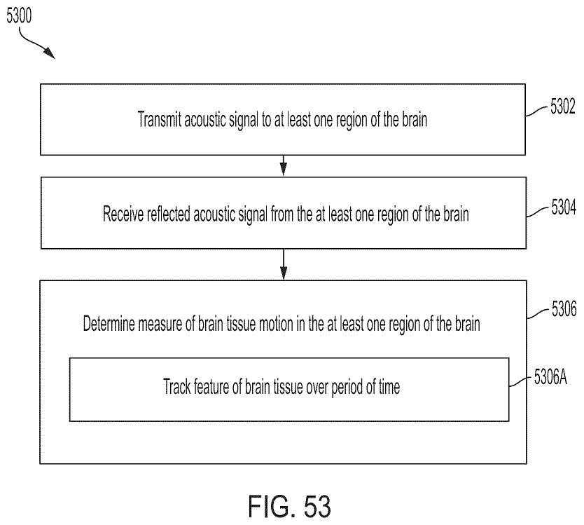

In some embodiments, one or more tissue tracking techniques may be used to extract brain beat signals is to track tissue movement. FIG. 53 shows an illustrative process for determining a measure of brain tissue motion in a brain via tissue tracking, according to some embodiments

The process 5300 may begin at act 5302. Acts 5302-5306 may be performed in the same manner as acts 4402-4406 of process 4400.

At act 5306A, the measure of brain tissue motion in the at least one region of the brain may be determined at least in part by tracking a feature of brain tissue over a period of time.

The brain is a three-dimensional structure, however, the typical brightness (B-mode) image can capture only a two-dimensional slice of that image at a time. Accordingly, in order to accurately perform tissue tracking, image features that are known have some representation in the imaging plane at all times must be tracked. For example, brain ventricles may provide a good landmark for this purpose.

In addition, tissue motion can be subtle and hence invisible due to low spatial resolution. Accordingly, the tissue tracking techniques described herein take into consideration techniques for overcoming the low spatial resolution of B-mode images which may render tissue motion imperceptible.

i. Finite Difference Techniques/Correlation of Consecutive Frames

As described herein, tracking the exact location of tissue may be difficult to achieve. As an alternative, in some embodiments, the image similarity and/or differences over time may be tracked.

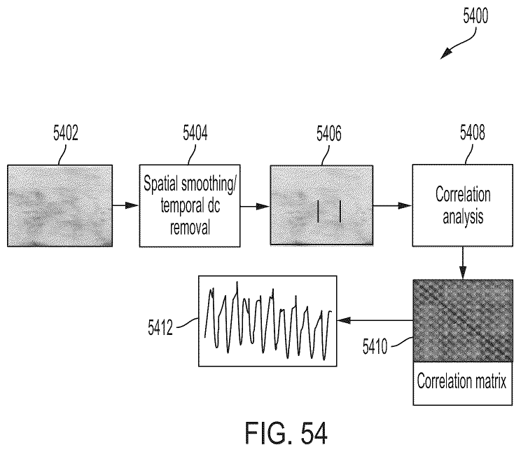

FIG. 54 shows an example flow diagram of tissue tracking using finite differences, according to some embodiments. The process illustrated in flow diagram 5400 may begin at act 5402 where a set of ultrasound images is obtained. The set of ultrasound images may comprise at least two images. In some embodiments, the set of ultrasound images comprises one or more B-mode images.

At act 5404, the set of ultrasound images is “cleaned” using spatial smoothing at every frame. In some embodiments, a de blocker may additionally or alternatively be used to remove the bias and shifts and drifts in the extracted signal.

At act 5406, a region of interest in the set of ultrasound images may be identified. The set of ultrasound images may be cut (e.g., cropped) based on the identified region of interest. As described herein, the region of interest may include features that are known to have some representation in the imaging plane at all times. For example, in some embodiments, the region of interest may include one or more ventricles.

At act 5408, a correlation matrix may be computed. For example, the correlation matrix may reflect a correlation between the set of ultrasound images. In some embodiments, the correlation matrix may reflect a correlation between behavior of a feature tracked over time through the set of ultrasound images.

At act 5410, a row of the correlation matrix may be selected as a beating representation of brain tissue depicted in the set of ultrasound images. The temporal oscillatory behavior of the tissue may be reflected in the structured correlation matrix. Beating follows an oscillatory behavior, which may be captured through comparing a template to the sequence of frames recorded in the set of B-mode images.

Accordingly, motion of the brain tissue may be extracted from the set of ultrasound images using the process 5400. Plot 5412 illustrates an example signal of brain tissue motion extracted from the set of ultrasound images by performing process 5400.

ii. Ventricle Beat Tracking

In some embodiments, a ventricle beat tracking technique may be implemented track tissue motion. For example, contraction and expansion of ventricles in the brain may be tracked. As mentioned before, tracking individual pixels may not be feasible in some instances to low spatial resolution. To overcome this, a ventricle's contractions and/or expansions may be tracked instead in order to capture the brain beat. In some embodiments, this ventricle tracking may be performed one-dimensionally, by measuring the distance between ventricle walls over time. In some embodiments, ventricle tracking may be performed two-dimensionally, by measuring changes in surface area of the ventricle. In some embodiments, ventricle tracking may be performed in three-dimensions, by measuring the ventricle volume.

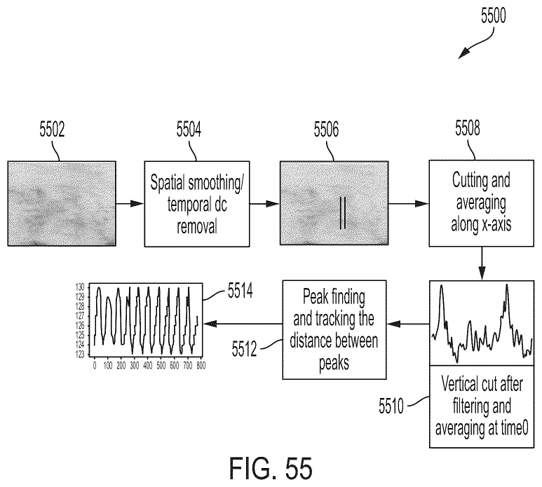

FIG. 55 shows an example flow diagram of ventricle edge tracking for brain beat extraction, according to some embodiments. FIG. 55 presents an initial assessment of the one-dimensional case for ventricle tracking. At act 5502, a set of ultrasound images is obtained. The set of ultrasound images may comprise at least two images. In some embodiments, the set of ultrasound images comprises one or more B-mode images.

At act 5504, the set of ultrasound images is “cleaned” using spatial smoothing at every frame. At act 5506, a dc blocker may be used to remove the bias and shifts and drifts in the extracted signal.

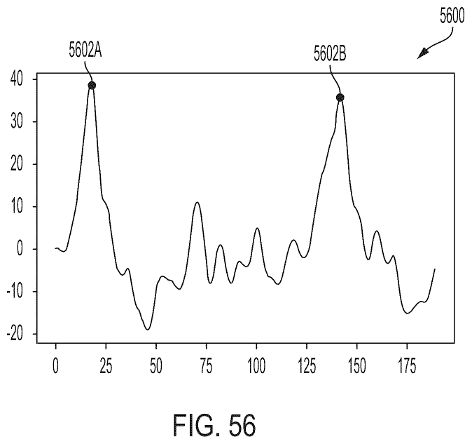

At act 5508, each frame is cut into segments that include the ventricle upper and lower wall. Multiple neighboring beams may be averaged to improve signal to noise ratio. In general, ventricle walls are relatively brighter than background. This leads to two peaks 5602A, 5602 in the extracted signal at every timestep which represent ventricle walls, as shown in the plot 5600 of FIG. 56. Plot 5600 mat be generated at act 5510. FIG. 56 shows an example of ventricle wall detection, according to some embodiments.

At act 5512, the distance between these peaks is tracked to extract the ventricle beat signal. The extracted wave shape here is in terms of beam sample depth, which is measurable in millimeters.

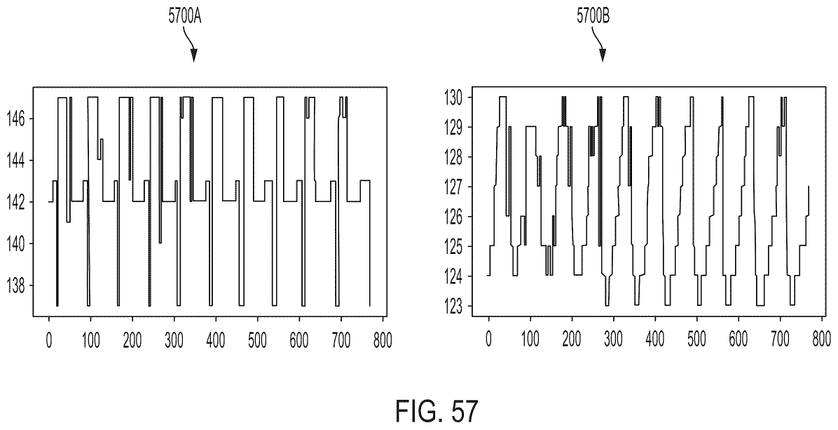

The number of beams selected to average at act 5508, has an adverse effect on extracted signal shape. Two examples 5700A, 5700B are shown in FIG. 57. FIG. 57 show examples 5700A, 5700B of sample beat signals extracted from different regions of the ventricle, according to some embodiments. Accordingly, in some embodiments, two-dimensional and/or three-dimensional ventricle tracking techniques which track surface area or volume are used which may lead to a more robust extraction of tissue motion.

d. Spectral Clustering

Finding patterns in B-mode frame sequences may be sensitive to spatial location. Groups of pixels in different spatial locations may have a synchronous behavior, while immediate neighbors might show a completely different pattern. Accordingly, averaging close by pixels to extract temporal patterns from B-mode frame sequences may not be possible.

Instead, described herein is a system that performs spatiotemporal clustering to group pixels together and extracts a spatial and temporal pattern in B-mode images from those clusters.

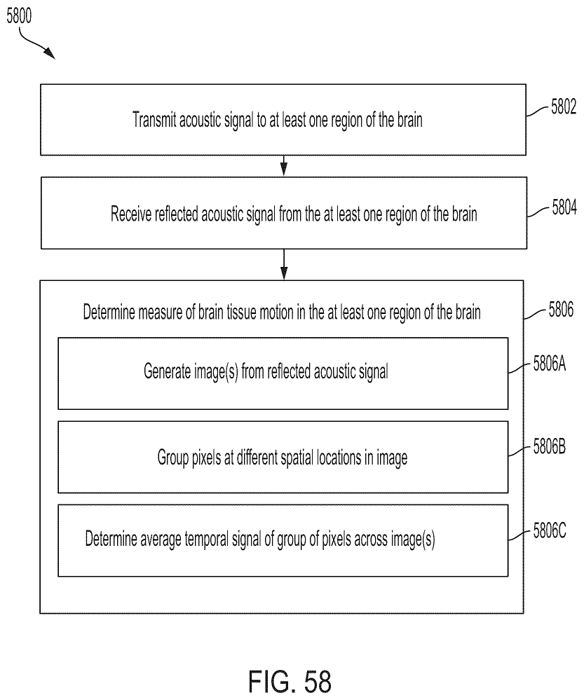

FIG. 58 shows an illustrative process for determining a measure of brain tissue motion in a brain via spectral clustering, according to some embodiments.

The process 5800 may begin at act 5802. Acts 5802-5806 may be performed in the same manner as acts 4402-4406 of process 4400.

At acts 5806A-5806C, the measure of brain tissue motion in the at least one region of the brain may be determined at least in part by performing spectral clustering. For example, as described herein, one or more images may be generated from the reflected acoustic signal at act 5806A. In some embodiments, the image(s) may comprise one or more B-mode ultrasound images.

At act 5806B, pixels of a respective image of the image(s) may be grouped together. The pixels may be located at different spatial locations in the image. In particular, the pixels grouped together may not be neighboring pixels. Instead, the pixels may be grouped based on their exhibiting the same behavior in the image.

At act 5806C, an average temporal signal of the group of pixels clustered together at act 5806B may be determined. In some embodiments, act 5806C may be performed for multiple groups of clustered pixels.

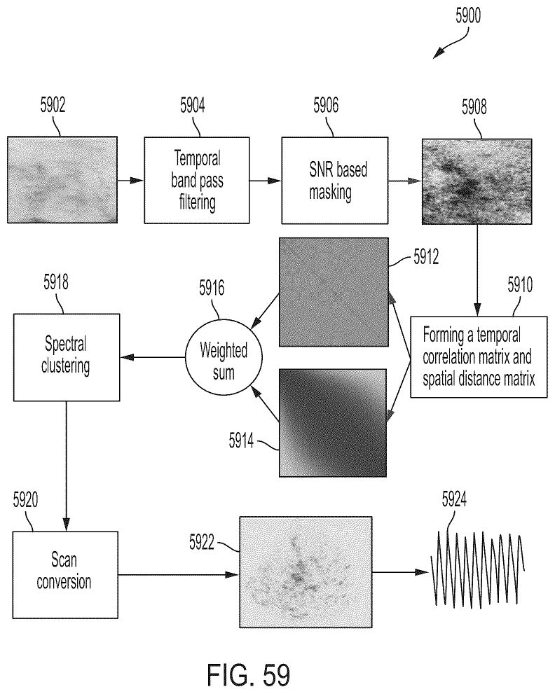

FIG. 59 shows an example flow diagram of spatiotemporal clustering, according to some embodiments.

To perform spatiotemporal clustering task, a set of ultrasound images may first be obtained at act 5902. The set of ultrasound images may comprise at least two images. In some embodiments, the set of ultrasound images may comprise one or more B-mode images.

At act 5904, the set of ultrasound images may be “cleaned” by applying a band pass filter to the signal. For example, in some embodiments, a bandpass filter with a passband of [0.3, 10] Hz may be applied.

At act 5906, the pixels may be masked using a signal to noise ratio (SNR) mask. Act 5908 shows the resulting images. It may be assumed that the signal of interest should have the maximum power in the frequency range of [0.3, 3] Hz.

At act 5910, a correlation matrix 5912 between different pixel time series may be estimated. A spatial distance matrix 5914 may also be computed at act 5910 to keep the pixels spatially contiguous. There is a tradeoff between the temporal correlation matrix and distance matrix, however this may be controlled by using a weighted sum, at act 5916.

At act 5918, spectral clustering may be performed. For example, pixels exhibiting a synchronous behavior may be clustered so that a temporal pattern can be extracted from the cluster.

At acts 5920-5922, averaged temporal signal for the cluster can be computed to estimate the brain beat signal. Plot 5924 illustrates the extracted motion signal.

In some embodiments, the spectral clustering techniques described herein may be used in combination with one or more other techniques. For example, in some embodiments, the spectral clustering techniques described herein may be used in combination with one or more of the signal decomposition techniques described herein (e.g., to decompose each cluster into temporal components).

### V. Example Applications of Pulsatility Mode Sensing

a. Brain Health Metrics

Pulsatility mode measurements obtained according to the techniques described herein may facilitate determination of a number of metrics that may be used to assess brain health. For example, in some embodiments, the pulsatility mode measurements obtained according to the techniques described herein may be used to determine a measure of intracranial pressure, cerebral blood flow velocity (CBFV), intracranial elastance and/or beating (pulsatility) of the brain.

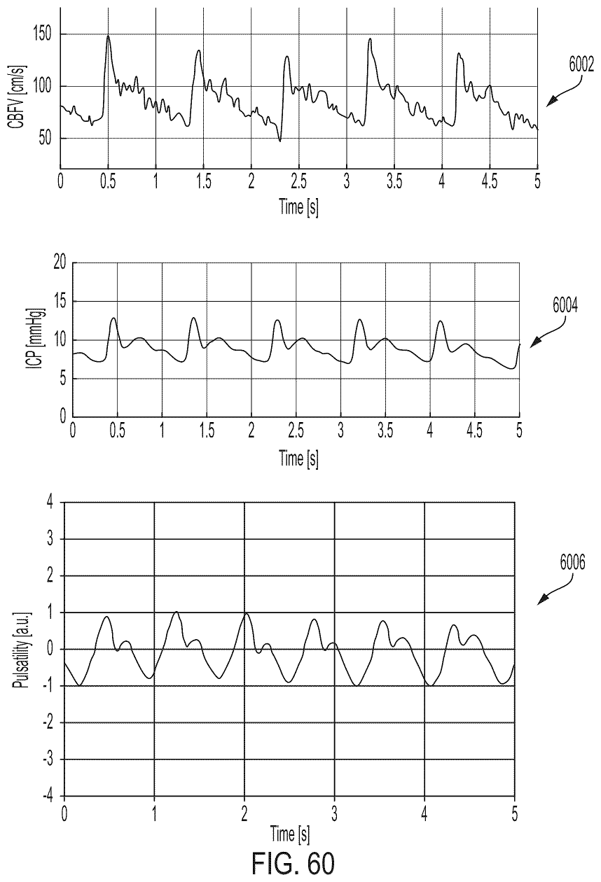

The techniques described herein assume that the heart acts as an endogenous mechanical driver that induces motion over the cardiac cycle in the brain. This motion subsequently leads to transient changes in the blood flow and pressure in the brain. These waveforms are synchronous with the arterial pulse. Exemplary data is shown in FIG. 60. In particular, FIG. 60 shows example plots of cerebral blood flow 6002, intracranial pressure 6004, and pulsatility 6006 waveforms, according to some embodiments.

In healthy subjects, ICP waveforms are trifid: there are three distinct peaks, which correlate to the arterial pressure. All these waves are rarely more than 4 mmHg in amplitude, or 10-30% of the mean ICP.

Changes in the shape of the ICP waveforms, correlate with different brain conditions. For example, increasing amplitude of all waveforms suggests rising intracranial pressure, decreasing amplitude of the PI waveform suggests decreased cerebral perfusion, increasing amplitude of the P2 waveform suggests decreased cerebral compliance. “Plateau” waves suggest intact cerebral blood flow autoregulation, etc.

These changes manifest in the form of low frequency tissue strain, which due to its dynamic nature, leads to different temporal patterns of pulsatility in brain tissue (aka tissue motion) and pulsatility in cerebral blood flow.

According to some aspects of the technology, there is provided methods for measuring the changes in the pulsatility behaviors and correlating them to metrics of brain health including ICP, cerebral blood flow, and ICE. Such an approach enables the characterization of brain tissue integrity in a wide range of neurological diseases in which changes in the biomechanical properties of the brain can lead to dramatic changes in pressure and flow dynamics, and hence tissue motion. Such techniques also provide a fast means of revealing subtle physiological variations of the brain and potentially other tissues.

In some embodiments, the methods may be performed using one or more machine learning algorithms. The machine learning algorithms can be in the form of a classification or regression algorithm, which may include one or more sub-components such as convolutional neural networks, recurrent neural networks such as LSTMs and GRUs, linear SVMs, kernel SVMs, linear and/or nonlinear regression, and various techniques from unsupervised learning such as variational autoencoders (VAE), generative adversarial networks (GANs) which are used to extract relevant features from the raw input data and partially supervised learning methods such as self-supervised learning, semi-supervised learning and reinforcement learning which learn the transfer functions either with limited labels or through extracting correlation and causality for existing data. FIG. 43 shows an example machine learning neural network 4300 that can be configured for extracting intracranial pressure using pulsatility mode sensing.

As shown in FIG. 43, the example machine learning neural network 4300 may include one or more layers. An input layer 4304 is provided for receiving input data 4302. When training the example machine learning neural network 4300, as described herein, the input 4302 may comprise training data. When using the example machine learning neural network 4300 to obtain new information, the input 4302 may comprise, in some embodiments, a spatiotemporal p-mode signal, as described herein. An output layer 4308 is provided for outputting information generated by the example machine learning neural network 4300.

Between the input layer 4304 and the output layer 4308, a number of additional layers may be provided. For example, one or more hidden layers 4306, one or more convolution and max pooling layers 4310, and one or more fully connected layers 4312 may be provided. The one or more fully connected layers 4312 may comprise multiple nodes, each node being connected to each node of the output layer 4308, as shown in FIG. 43.

The techniques described herein may use training data collected from a cohort of patients. The data may be used to “train” the machine learning model. This model may then be used to infer where to optimally steer an ultrasound beam and detect, monitor, or localize brain conditions during the “test” time. The same model may be further employed with techniques such as reinforcement learning to continuously learn and adapt to a patient's normal and abnormal brain activities. In some aspects, the training data can be generated using machine learning techniques such as VAE and GANS and/or physics based in-silico (simulation-based) models.

In some embodiments, input for the machine learning model may be the spatiotemporal p-mode signals obtained according to the techniques described herein, or features extracted thereof. The model may output a performance metric constructed based on the numeric values and time-waveforms of benchmark ICP-ICE data (e.g., invasive ICP sensors).

b. Epilepsy and Seizure

In some embodiments, pulsatility mode measurements may be used to predict, monitor, and/or treat Epilepsy and seizures. Epilepsy is a group of neurological disorders characterized by epileptic seizures. Epileptic seizures are episodes that can vary from brief and nearly undetectable periods to long periods of vigorous shaking. These episodes can result in physical injuries, including occasionally broken bones. In epilepsy, seizures tend to recur and have no immediate underlying cause. The cause of most cases of epilepsy is unknown. Some cases occur as the result of brain injury, stroke, brain tumors, infections of the brain, and birth defects through a process known as epileptogenesis. Epileptic seizures are the result of excessive and abnormal neuronal activity in the cortex of the brain. The diagnosis involves ruling out other conditions that might cause similar symptoms, such as fainting, and determining if another cause of seizures is present, such as alcohol withdrawal or electrolyte problems. This may be partly done by imaging the brain and performing blood tests. Epilepsy can often be confirmed with an electroencephalogram (EEG).

The diagnosis of epilepsy is typically made based on observation of the seizure onset and the underlying cause. An electroencephalogram (EEG) to look for abnormal patterns of brain waves and neuroimaging (CT scan or MRI) to look at the structure of the brain are also usually part of the workup. While figuring out a specific epileptic syndrome is often attempted, it is not always possible. Video and EEG monitoring may be useful in difficult cases. An electroencephalogram (EEG) can assist in showing brain activity suggestive of an increased risk of seizures. It is only recommended for those who are likely to have had an epileptic seizure on the basis of symptoms. In the diagnosis of epilepsy, electroencephalography may help distinguish the type of seizure or syndrome present.

Accordingly, in some embodiments, pulsatility mode measurements may be used in addition or alternative to the methods described herein for predicting, monitoring, and/or treating Epilepsy and seizures. Pulsatility mode measurements provide a more accurate, cost-efficient, and non-invasive method of predicting, monitoring, and/or treating Epilepsy and seizures.

### Computer System

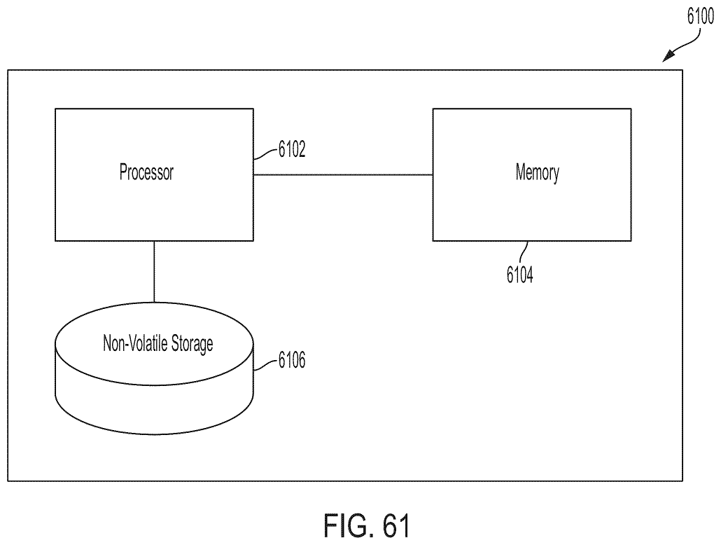

FIG. 61 shows a block diagram of an example computer system 6100 that may be used to implement embodiments of the technology described herein. The computing device 6100 may include one or more computer hardware processors 6102 and non-transitory computer-readable storage media (e.g., memory 6104 and one or more non-volatile storage devices 6106). The processor(s) 6102 may control writing data to and reading data from (1) the memory 6104; and (2) the non-volatile storage device(s) 6106. To perform any of the functionality described herein, the processor(s) 6102 may execute one or more processor-executable instructions stored in one or more non-transitory computer-readable storage media (e.g., the memory 6104), which may serve as non-transitory computer-readable storage media storing processor-executable instructions for execution by the processor(s) 6102.

The terms “program” or “software” are used herein in a generic sense to refer to any type of computer code or set of processor-executable instructions that can be employed to program a computer or other processor (physical or virtual) to implement various aspects of embodiments as discussed above. Additionally, according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform tasks or implement abstract data types. Typically, the functionality of the program modules may be combined or distributed.

Various concepts described herein may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, for example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term). The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

### Summary of Some Embodiments

According to some embodiments, a method of determining intracranial pressure (ICP) of a subject's brain is provided. The method comprises: obtaining acoustic measurement data obtained from measuring acoustic signals from the subject's brain; determining a cerebral blood flow velocity (CBFV) measurement of the subject's brain using the acoustic measurement data; obtaining an arterial blood pressure (ABP) measurement of the subject's brain; generating, using the CBFV measurement and the ABP measurement, input to a machine learning model trained to output an ICP measurement; and providing the input to the machine learning model to obtain an ICP measurement of the subject's brain.

In some embodiments. the machine learning model includes a first convolutional network and a second convolutional network; and providing the input to the machine learning model to obtain the ICP measurement of the subject's brain comprises: providing first input generated from the CBFV measurement to the first convolutional neural network to obtain a first output; providing second input generated from the ABP measurement to the second convolutional neural network to obtain a second output; and determining the ICP measurement of the subject's brain using the first and second outputs. In some embodiments, determining the ICP measurement of the subject's brain using the first and second outputs comprises: generating a combined input for an ICP predictor of the machine learning model using the first and second outputs; and providing the combined input to the ICP predictor to obtain the ICP measurement of the subject's brain. In some embodiments, the first output is a first ICP measurement and the second output is a second ICP measurement, and determining the ICP measurement of the subject's brain using the first and second outputs comprises: performing a comparison between the first and second outputs to determine the ICP measurement of the subject's brain.

In some embodiments, the acoustic measurement data is obtained by: guiding an acoustic beam towards a region of the subject's brain; and detecting a signal from the region of interest of the subject's brain.

In some embodiments, the machine learning model comprises a contrastive convolutional network. In some embodiments, the machine learning model comprises a decision tree model.

In some embodiments, generating, using the CBFV measurement and the ABP measurement, the input to a physics guided machine learning model comprises: determining, using the CBFV measurement, a mean CBFV value as an input; and determining, using the ABP measurement, a mean ABP value as an input.

In some embodiments, the CBFV measurement comprises a time series of CBFV values;

the ABP measurement comprises a time series of ABP values; and generating, using the CBFV measurement and the ABP measurement, the input to the physics guided machine learning model comprises: identifying one or more characteristics of the time series of CBFV values and/or one or more characteristics of the time series of the ABP values; and generating, using the one or more characteristics of the time series of CBFV values and/or the one or more characteristics of the time series of ABP values, the input to the physics guided machine learning model.

In some embodiments, generating, using the CBFV measurement and the ABP measurement, the input to a physics guided machine learning model comprises: determining frequency domain CBFV values using the CBFV measurement; determining frequency domain ABP values using the ABP measurement; determining a mean CBFV value using the frequency domain CBFV values; determining a mean ABP value using the frequency domain ABP values; and generating the input using the mean CBFV value and the mean ABP value.

In some embodiments, the machine learning model comprises a model based on a resistor capacitor (RC) circuit model of the subject's brain.

In some embodiments, the ICP measurement of the subject's brain is a mean ICP value. In some embodiments, the ICP measurement of the subject's brain is a time series of ICP values.

According to some embodiments, an ICP measurement system is provided. The ICP measurement system comprises: one or more probes configured to obtain acoustic measurement data by detecting acoustic signals in a subject's brain; and at least one computer hardware processor configured to: determine a cerebral blood flow velocity (CBFV) measurement of the subject's brain using the acoustic measurement data; obtain an arterial blood pressure (ABP) measurement of the subject's brain; generate, using the CBFV measurement and the ABP measurement, input to a machine learning model trained to output an ICP measurement; and provide the input to the machine learning model to obtain an ICP measurement of the subject's brain.

According to some embodiments, at least one non-transitory computer-readable storage medium storing instructions is provided. The instructions, when executed by at least one computer hardware processor, cause the at least one computer hardware processor to perform: obtaining acoustic measurement data obtained from measuring acoustic signals from the subject's brain; determining a cerebral blood flow velocity (CBFV) measurement of the subject's brain using the acoustic measurement data; obtaining an arterial blood pressure (ABP) measurement of the subject's brain; generating, using the CBFV measurement and the ABP measurement, input to a machine learning model trained to output an ICP measurement; and providing the input to the machine learning model to obtain an ICP measurement of the subject's brain.

According to some embodiments, a method of determining intracranial pressure (ICP) of a subject's brain is provided. The method comprises: obtaining acoustic measurement data from detecting acoustic signals from the subject's brain; determining an arterial blood pressure (ABP) measurement of the subject's brain using the acoustic measurement data; determining a cerebral blood flow (CBF) measurement using the acoustic measurement data; and determining an ICP measurement of the subject's brain using the CBF measurement and the ABP measurement, wherein determining the ICP measurement comprises using a physics guided machine learning model to obtain the ICP measurement of the subject's brain.

In some embodiments, determining the ABP measurement of the subject using the acoustic measurement data comprises using a first machine learning model to obtain the ABP measurement. In some embodiments, the first machine learning model comprises an encoder and a decoder. In some embodiments, using the first machine learning model to obtain the ABP measurement comprises:

generating a first input using the acoustic measurement data; providing the first input to the encoder to obtain a latent representation of the acoustic measurement data; and generating a second input using the latent representation of the acoustic measurement data; and providing the second input to the decoder to obtain the ABP measurement. In some embodiments, the latent representation of the acoustic measurement data comprises a probability distribution and generating the second input using the latent representation of the acoustic measurement data comprises sampling the second input from the probability distribution. In some embodiments, the ABP measurement comprises an ABP sample corresponding to the sampled second input.

In some embodiments, the method further comprises receiving contextual data about the subject, wherein determining the ABP measurement of the subject's brain comprises using the contextual data to determine the ABP measurement. In some embodiments. the contextual data comprises a mean ABP measurement, a vessel diameter, the subject's age, the subject's gender, and/or one or more dimensions of the subject's skull.

In some embodiments, an ICP measurement device is provided. The ICP measurement device comprises: one or more probes configured to obtain acoustic measurement data by detecting acoustic signals in a subject's brain; and a computer hardware processor configured to: determine an arterial blood pressure (ABP) measurement of the subject's brain using the acoustic measurement data; determine a cerebral blood flow (CBF) measurement using the acoustic measurement data; and determine an ICP measurement of the subject's brain using the CBF measurement and the ABP measurement, wherein determining the ICP measurement comprises using a physics guided machine learning model to obtain the ICP measurement of the subject's brain.

In some embodiments, determining the ABP measurement of the subject using the acoustic measurement data comprises using a first machine learning model to obtain the ABP measurement. In some embodiments, the first machine learning model comprises an encoder and a decoder. In some embodiments, using the first machine learning model to obtain the ABP measurement comprises: generating a first input using the acoustic measurement data; providing the first input to the encoder to obtain a latent representation of the acoustic measurement data; and generating a second input using the latent representation of the acoustic measurement data; and providing the second input to the decoder to obtain the ABP measurement. In some embodiments, the latent representation of the acoustic measurement data comprises a probability distribution and generating the second input using the latent representation of the acoustic measurement data comprises sampling the second input from the probability distribution.

In some embodiments, the ABP measurement comprises an ABP sample corresponding to the sampled second input. In some embodiments, the processor is further configured to: receive contextual data about the subject, wherein determining the ABP measurement of the subject's brain comprises using the contextual data to determine the ABP measurement. In some embodiments, the contextual data comprises a mean ABP measurement, a vessel diameter, the subject's age, the subject's gender, and/or one or more dimensions of the subject's skull.

According to some embodiments, a non-transitory computer-readable storage medium storing instructions is provided. The instructions, when executed by a processor, cause the processor to perform: determining an arterial blood pressure (ABP) measurement of the subject's brain using acoustic measurement data obtained from measuring acoustic signals from a subject's brain; determining a cerebral blood flow (CBF) measurement using the acoustic measurement data; and determining an ICP measurement of the subject's brain using the CBF measurement and the ABP measurement, wherein determining the ICP measurement comprises using a physics guided machine learning model to obtain the ICP measurement of the subject's brain.

In some embodiments, determining the ABP measurement of the subject using the acoustic measurement data comprises using a first machine learning model to obtain the ABP measurement. In some embodiments, the first machine learning model comprises an encoder and a decoder.

In some embodiments, using the first machine learning model to obtain the ABP measurement comprises: generating a first input using the acoustic measurement data; providing the first input to the encoder to obtain a latent representation of the acoustic measurement data; and generating a second input using the latent representation of the acoustic measurement data; and providing the second input to the decoder to obtain the ABP measurement.

According to some embodiments, a method of determining intracranial pressure (ICP) of a subject's brain is provided. The method comprises: obtaining acoustic measurement data and pulsatility data from detecting acoustic signals from the subject's brain; determining a measure of brain perfusion using the acoustic measurement data and the pulsatility data; and determining an ICP measurement of the subject's brain using the measure of brain perfusion, wherein determining the ICP measurement comprises using a physics guided machine learning model to obtain the ICP measurement of the subject's brain.

In some embodiments, the acoustic measurement data is obtained by: guiding an acoustic beam towards a region of the subject's brain; and detecting a signal from the region of interest of the subject's brain. In some embodiments, the acoustic measurement data comprises quantitative ultrasound (QUS) data. In some embodiments, the QUS data is obtained by: guiding an acoustic beam towards a region of the subject's brain; and detecting a signal from the region of interest of the subject's brain.

In some embodiments, the method further comprises determining an ABP measurement of the subject's brain using the acoustic measurement data, wherein determining the ICP measurement of the subject's brain comprises using the ABP measurement to determine the ICP measurement. In some embodiments, determining the ABP measurement using the acoustic measurement data comprises: determining, using the acoustic measurement data, input to a machine learning model; and providing the input to the machine learning model to obtain output indicating the ABP measurement. In some embodiments, the machine learning model comprises an encoder and a decoder.

According to some embodiments, an ICP measurement device is provided. The ICP measurement device comprises: one or more probes configured to obtain acoustic measurement data and pulsatility data by detecting acoustic signals from a subject's brain; and a processor configured to: determine measure of brain perfusion using the acoustic measurement data and the pulsatility data; and determine an ICP measurement of the subject's brain using the measure of brain perfusion, wherein determining the ICP measurement comprises using a physics guided machine learning model to obtain the ICP measurement of the subject's brain.

In some embodiments, the acoustic measurement data is obtained by: guiding an acoustic beam towards a region of the subject's brain; and detecting, by the one or more probes, a signal from the region of interest of the subject's brain. In some embodiments, the acoustic measurement data comprises quantitative ultrasound (QUS) data. In some embodiments, the QUS data is obtained by: guiding an acoustic beam towards a region of the subject's brain; and detecting a signal from the region of interest of the subject's brain.

In some embodiments, the processor is further configured to determine an ABP measurement of the subject's brain using the acoustic measurement data, wherein determining the ICP measurement of the subject's brain comprises using the ABP measurement to determine the ICP measurement. In some embodiments, determining the ABP measurement using the acoustic measurement data comprises: determining, using the acoustic measurement data, input to a machine learning model; and providing the input to the machine learning model to obtain output indicating the ABP measurement. In some embodiments, the machine learning model comprises an encoder and a decoder.

According to some embodiments, a non-transitory computer-readable storage medium storing instructions is provided. The instructions, when executed by a processor, cause the processor to perform: determining a measure of brain perfusion using acoustic measurement data and pulsatility data obtained from measuring acoustic signals from a subject's brain; and determining an ICP measurement of the subject's brain using the measure of brain perfusion, wherein determining the ICP measurement comprises using a physics guided machine learning model to obtain the ICP measurement of the subject's brain.

In some embodiments, the acoustic measurement data is obtained by: guiding an acoustic beam towards a region of the subject's brain; and detecting a signal from the region of interest of the subject's brain. In some embodiments, the acoustic measurement data comprises quantitative ultrasound (QUS) data. In some embodiments, the QUS data is obtained by: guiding an acoustic beam towards a region of the subject's brain; and detecting a signal from the region of interest of the subject's brain.

In some embodiments, the instructions further cause the processor to perform determining an ABP measurement of the subject's brain using the acoustic measurement data, wherein determining the ICP measurement of the subject's brain comprises using the ABP measurement to determine the ICP measurement.

In some embodiments, determining the ABP measurement using the acoustic measurement data comprises: determining, using the acoustic measurement data, input to a machine learning model; and providing the input to the machine learning model to obtain output indicating the ABP measurement.

In some embodiments, a method of determining intracranial pressure (ICP) of a subject's brain is provided. The method comprises: obtaining acoustic measurement data obtained from measuring acoustic signals from the subject's brain; determining, using the acoustic measurement data, a ventricle deformation measurement of the subject's brain; and determining an ICP measurement of the subject's brain using the ventricle deformation measurement of the subject's brain, wherein determining the ICP measurement comprises using a physics guided machine learning model to obtain the ICP measurement of the subject's brain.

In some embodiments, the acoustic measurement data is obtained by: guiding an acoustic beam towards a region of the subject's brain; and detecting a signal from the region of interest of the subject's brain.

In some embodiments, determining the ventricle deformation measurement comprises obtaining a measurement of contraction and expansion of a ventricle in the subject's brain during one or more cardiac cycles.

In some embodiments, determining the ventricle deformation measurement comprises:
determining a change in distance between ventricle walls; and determining the ventricle deformation measurement based on the change in distance between ventricle walls.

In some embodiments, determining the ventricle deformation measurement comprises:

determining a change in surface area of a ventricle; and determining the ventricle deformation measurement based on the change in surface area of the ventricle.

In some embodiments, determining the ventricle deformation measurement comprises:

determining a change in volume of a ventricle; and determining the ventricle deformation measurement based on the change in volume of the ventricle.

In some embodiments, the physics guided machine learning model is based on an elasticity model of the subject's brain. In some embodiments, the elasticity model is a Saint Venant-Kirchhoff model.

In some embodiments, using the physics guided machine learning model to obtain the ICP measurement of the subject's brain comprises: generating, using the ventricle deformation measurement of the subject's brain, first input for a first model of the physics guided machine learning model; providing the first input to the first model to obtain a representation of ventricle elasticity; and determining the ICP measurement of the subject's brain using the representation of ventricle elasticity.

According to some embodiments, an ICP measurement device is provided. T ICP measurement device comprises: one or more probes configured to obtain acoustic measurement data from detecting acoustic signals in a subject's brain; and a processor configured to: determine, using the acoustic measurement data, a ventricle deformation measurement of the subject's brain; and determine an ICP measurement of the subject's brain using the ventricle deformation measurement of the subject's brain, wherein determining the ICP measurement comprises using a physics guided machine learning model to obtain the ICP measurement of the subject's brain.

In some embodiments, the acoustic measurement data is obtained by guiding an acoustic beam towards a region of the subject's brain; and detecting a signal from the region of interest of the subject's brain.

In some embodiments, determining the ventricle deformation measurement comprises determining a measurement of contraction and expansion of a ventricle in the subject's brain during one or more cardiac cycles.

In some embodiments, determining the ventricle deformation measurement comprises:

determining a change in distance between ventricle walls; and determining the ventricle deformation measurement based on the change in distance between ventricle walls.

In some embodiments, determining the ventricle deformation measurement comprises:

determining a change in surface area of a ventricle; and determining the ventricle deformation measurement based on the change in surface area of the ventricle.

In some embodiments, determining the ventricle deformation measurement comprises:

determining a change in volume of a ventricle; and determining the ventricle deformation measurement based on the change in volume of the ventricle.

In some embodiments, the physics guided machine learning model is based on an elasticity model of the subject's brain. In some embodiments, the elasticity model is a Saint Venant-Kirchhoff model.

In some embodiments, using the physics guided machine learning model to obtain the ICP measurement of the subject's brain comprises: generating, using the ventricle deformation measurement of the subject's brain, first input for a first model of the physics guided machine learning model; providing the first input to the first model to obtain a representation of ventricle elasticity; and determining the ICP measurement of the subject's brain using the representation of ventricle elasticity.

In some embodiments, a non-transitory computer-readable storage medium storing instructions that, when executed by a processor, cause the processor to perform: determining, using acoustic measurement data obtained from measuring acoustic signals from a subject's brain, a ventricle deformation measurement of the subject's brain; and determining an ICP measurement of the subject's brain using the ventricle deformation measurement of the subject's brain, wherein determining the ICP measurement comprises using a physics guided machine learning model to obtain the ICP measurement of the subject's brain.

In some embodiments, determining the ventricle deformation measurement comprises obtaining a measurement of contraction and expansion of a ventricle in the subject's brain during one or more cardiac cycles.

In some embodiments, a method of determining arterial blood pressure (ABP) in a subject's brain is provided. The method comprises: obtaining acoustic measurement data obtained from detecting acoustic signals from the subject's brain; determining, using the acoustic measurement data, an arterial deformation measurement of the subject's brain; and determining an ABP measurement of the subject's brain using the arterial deformation measurement of the subject's brain, wherein determining the ABP measurement comprises using a physics guided machine learning model to obtain the ABP measurement of the subject's brain.

In some embodiments, the acoustic measurement data is obtained by: guiding an acoustic beam towards a region of the subject's brain; and detecting a signal from the region of interest of the subject's brain.

In some embodiments, determining the arterial deformation measurement comprises obtaining a measurement of contraction and expansion of an artery in the subject's brain during one or more cardiac cycles.

In some embodiments, determining the arterial deformation measurement comprises: determining a change in distance between artery walls; and determining the arterial deformation measurement based on the change in distance between artery walls.

In some embodiments, determining the arterial deformation measurement comprises: determining a change in cross-sectional surface area of an artery; and determining the arterial deformation measurement based on the change in cross-sectional surface area of the artery.

In some embodiments, determining the arterial deformation measurement comprises:

determining a change in volume of at least a portion of an artery; and determining the arterial deformation measurement based on the change in volume of the at least a portion of the artery.

In some embodiments, the physics guided machine learning model is based on an elasticity model of the subject's brain. In some embodiments, the elasticity model is a Saint Venant-Kirchhoff model.

In some embodiments, using the physics guided machine learning model to obtain the ABP measurement of the subject's brain comprises: generating, using the arterial deformation measurement of the subject's brain, first input for a first model of the physics guided machine learning model; providing the first input to the first model to obtain a representation of arterial elasticity; and determining the ABP measurement of the subject's brain using the representation of arterial elasticity.

In some embodiments, an ABP measurement device is provided. The ABP measurement device comprises: one or more probes configured to obtain acoustic measurement data from detecting acoustic signals from a subject's brain; and a processor configured to: determine, using the acoustic measurement data, an arterial deformation measurement of the subject's brain; and determine an ABP measurement of the subject's brain using the arterial deformation measurement of the subject's brain, wherein determining the ABP measurement comprises using a physics guided machine learning model to obtain the ABP measurement of the subject's brain.

In some embodiments, the acoustic measurement data is obtained by: uiding an acoustic beam towards a region of the subject's brain; and detecting a signal from the region of interest of the subject's brain.

In some embodiments, determining the arterial deformation measurement comprises determining a measurement of contraction and expansion of an artery in the subject's brain during one or more cardiac cycles.

In some embodiments, determining the arterial deformation measurement comprises: determining a change in distance between artery walls; and determining the arterial deformation measurement based on the change in distance between artery walls.

In some embodiments, determining the arterial deformation measurement comprises: determining a change in cross-sectional surface area of an artery; and determining the arterial deformation measurement based on the change in cross-sectional surface area of the artery.

In some embodiments, determining the arterial deformation measurement comprises: determining a change in volume of at least a portion of an artery; and determining the arterial deformation measurement based on the change in volume of the at least a portion of the artery.

In some embodiments, the physics guided machine learning model is based on an elasticity model of the subject's brain. In some embodiments, the elasticity model is a Saint Venant-Kirchhoff model.

In some embodiments, using the physics guided machine learning model to obtain the ABP measurement of the subject's brain comprises: generating, using the arterial deformation measurement of the subject's brain, first input for a first model of the physics guided machine learning model; providing the first input to the first model to obtain a representation of arterial elasticity; and determining the ABP measurement of the subject's brain using the representation of arterial elasticity.

In some embodiments, a non-transitory computer-readable storage medium storing instructions that is provided. The instructions, when executed by a processor, cause the processor to perform: determining, using acoustic measurement data obtained from measuring acoustic signals from a subject's brain, an arterial deformation measurement of the subject's brain; and determining an ABP measurement of the subject's brain using the arterial deformation measurement of the subject's brain, wherein determining the ABP measurement comprises using a physics guided machine learning model to obtain the ABP measurement of the subject's brain.

In some embodiments, wherein determining the arterial deformation measurement comprises obtaining a measurement of contraction and expansion of an artery in the subject's brain during one or more cardiac cycles.

According to some embodiments, a method of determining arterial elastance in a subject's brain is provided. The method comprises: obtaining acoustic measurement data obtained from detecting acoustic signals from the subject's brain; determining, using the acoustic measurement data, an arterial deformation measurement of an artery in the subject's brain; and determining an arterial elastance measurement for the subject's brain using the arterial deformation measurement.

In some embodiments, determining the arterial elastance measurement comprises determining a pulse wave velocity (PWV); and determining the arterial elastance measurement using the arterial deformation measurement comprises determining the arterial elastance measurement using the PWV. In some embodiments, determining the PWV comprises: determining a distance between two points in an artery; determining a pulse transit time (PTT); and determining the PWV using the distance between the two points in the artery and the PTT. In some embodiments, the acoustic measurement data is obtained by: guiding an acoustic beam towards a region of the subject's brain; and detecting a signal from the region of interest of the subject's brain.

In some embodiments, determining the arterial elastance measurement using the arterial deformation measurement comprises using a machine learning model to determine the arterial elastance measurement. In some embodiments, using the machine learning model to determine the measurement of arterial elastance comprises: generating, using the arterial deformation measurement, input to the machine learning model; providing the input to the machine learning model to obtain the arterial elastance measurement. In some embodiments, the machine learning model comprises a neural network.

In some embodiments, an arterial elastance measurement device is provided. The arterial elastance measurement device comprises: one or more probes configured to obtain acoustic measurement data from detecting acoustic signals from a subject's brain; and a processor configured to: determine, using the acoustic measurement data, an arterial deformation measurement of an artery in the subject's brain; and determine an arterial elastance measurement for the subject's brain using the arterial deformation measurement.

In some embodiments, processor is further configured to: determine the arterial elastance measurement comprises determining a pulse wave velocity (PWV); and determine the arterial elastance measurement using the arterial deformation measurement comprises determining the arterial elastance measurement using the PWV. In some embodiments, determining the PWV comprises: determining a distance between two points in an artery; determining a pulse transit time (PTT); and determine the PWV using the distance between the two points in the artery and the PTT. In some embodiments, the acoustic measurement data is obtained by: guiding an acoustic beam towards a region of the subject's brain; and detecting a signal from the region of interest of the subject's brain.

In some embodiments, determining the arterial elastance measurement using the arterial deformation measurement comprises using a machine learning model to determine the arterial elastance measurement. In some embodiments, using the machine learning model to determine the measurement of arterial elastance comprises: generating, using the arterial deformation measurement, input to the machine learning model; providing the input to the machine learning model to obtain the arterial elastance measurement. In some embodiments, the machine learning model comprises a neural network.

In some embodiments, a non-transitory computer-readable storage medium storing instructions is provided. The instructions, when executed by a processor, cause the processor to perform: determining, using acoustic measurement data obtained from measuring acoustic signals from a subject's brain, an arterial deformation measurement of an artery in the subject's brain; and determining an arterial elastance measurement for the subject's brain using the arterial deformation measurement.

In some embodiments, determining the arterial elastance measurement comprises determining a pulse wave velocity (PWV); and determining the arterial elastance measurement using the arterial deformation measurement comprises determining the arterial elastance measurement using the PWV.

In some embodiments, determining the PWV comprises: determining a distance between two points in an artery; determining a pulse transit time (PTT); and determine the PWV using the distance between the two points in the artery and the PTT. In some embodiments, the acoustic measurement data is obtained by: guiding an acoustic beam towards a region of the subject's brain; and detecting a signal from the region of interest of the subject's brain.

In some embodiments, determining the arterial elastance measurement using the arterial deformation measurement comprises using a machine learning model to determine the arterial elastance measurement. In some embodiments, using the machine learning model to determine the measurement of arterial elastance comprises: generating, using the arterial deformation measurement, input to the machine learning model; providing the input to the machine learning model to obtain the arterial elastance measurement.

According to some embodiments, a method of determining intracranial elastance (ICE) of a subject's brain is provided. The method comprises: obtaining acoustic measurement data obtained from detecting acoustic signals from the subject's brain; determining, using the acoustic measurement data, a measurement of movement of one or more tissue areas in the subject's brain; and determining an intracranial elastance measurement of the subject's brain based on the measurement of movement of the one or more tissue areas in the subject's brain.

In some embodiments, determining the measurement of movement of the one or more tissue areas in the subject's brain comprises: obtaining a first waveform of brain movement at the one or more tissue areas at a first time; obtaining a second waveform of brain movement at the one or more tissue areas at a second time; and determining the measurement of movement using the first waveform and the second waveform. In some embodiments, determining the intracranial elastance measurement of the subject's brain based on the measurement of movement of the one or more tissue areas in the subject's brain comprises determining a change in phase between the first waveform and the second waveform.

In some embodiments, determining the measure of movement of the one or more tissue areas in the subject's brain comprises tracking the movement using information obtained by: transmitting an acoustic signal to a region of the subject's brain; and processing a subsequent acoustic signal received from the region of the subject's brain.

According to some embodiments, a device for measuring intracranial elastance in a subject's brain is provided. The device comprises: one or more probes configured to obtain acoustic measurement data from detecting acoustic signals from a subject's brain; and a processor configured to: determine, using the acoustic measurement data, a measurement of movement of one or more tissue areas in the subject's brain; and determine an intracranial elastance measurement of the subject's brain based on the measurement of movement of the one or more tissue areas in the subject's brain.

In some embodiments, determining the measurement of movement of the one or more tissue areas in the subject's brain comprises: obtaining a first waveform of brain movement at the one or more tissue areas at a first time; obtaining a second waveform of brain movement at the one or more tissue areas at a second time; and determining the measurement of movement using the first waveform and the second waveform. In some embodiments, determining the intracranial elastance measurement of the subject's brain based on the measurement of movement of the one or more tissue areas in the subject's brain comprises determining a change in phase between the first waveform and the second waveform. In some embodiments, determining the measure of movement of the one or more tissue areas in the subject's brain comprises tracking the movement using information obtained by: transmitting an acoustic signal to a region of the subject's brain; and processing a subsequent acoustic signal received from the region of the subject's brain.

According to some embodiments, a non-transitory computer-readable storage medium storing instructions is provided. The instructions, when executed by a processor, cause the processor to perform: determining, using acoustic measurement data obtained from measuring acoustic signals from a subject's brain, a measurement of movement of one or more tissue areas in the subject's brain; and determining an intracranial elastance measurement of the subject's brain based on the measurement of movement of the one or more tissue areas in the subject's brain.

In some embodiments, determining the measurement of movement of the one or more tissue areas in the subject's brain comprises: obtaining a first waveform of brain movement at the one or more tissue areas at a first time; obtaining a second waveform of brain movement at the one or more tissue areas at a second time; and determining the measurement of movement using the first waveform and the second waveform. In some embodiments, determining the intracranial elastance measurement of the subject's brain based on the measurement of movement of the one or more tissue areas in the subject's brain comprises determining a change in phase between the first waveform and the second waveform.

In some embodiments, determining the measure of movement of the one or more tissue areas in the subject's brain comprises tracking the movement using information obtained by: transmitting an acoustic signal to a region of the subject's brain; and processing a subsequent acoustic signal received from the region of the subject's brain.

What is claimed is:

1. A method of determining intracranial pressure (ICP) of a subject's brain, the method comprising:
using at least one computer hardware processor to perform:
obtaining acoustic measurement data obtained from measuring acoustic signals from the subject's brain;
determining a cerebral blood flow velocity (CBFV) measurement of the subject's brain using the acoustic measurement data;
obtaining an arterial blood pressure (ABP) measurement of the subject's brain;
generating, using the CBFV measurement and the ABP measurement, input to a machine learning model trained to output an ICP measurement; and
providing the input to the machine learning model to obtain an ICP measurement of the subject's brain.

2. The method of claim 1, wherein:
the machine learning model includes a first convolutional network and a second convolutional network; and
providing the input to the machine learning model to obtain the ICP measurement of the subject's brain comprises:
providing first input generated from the CBFV measurement to the first convolutional neural network to obtain a first output;
providing second input generated from the ABP measurement to the second convolutional neural network to obtain a second output; and
determining the ICP measurement of the subject's brain using the first and second outputs.

3. The method of claim 2, wherein determining the ICP measurement of the subject's brain using the first and second outputs comprises:
generating a combined input for an ICP predictor of the machine learning model using the first and second outputs; and
providing the combined input to the ICP predictor to obtain the ICP measurement of the subject's brain.

4. The method of claim 2, wherein the first output is a first ICP measurement and the second output is a second ICP measurement, and determining the ICP measurement of the subject's brain using the first and second outputs comprises:
performing a comparison between the first and second outputs to determine the ICP measurement of the subject's brain.

5. The method of claim 1, wherein the acoustic measurement data is obtained by:
guiding an acoustic beam towards a region of the subject's brain; and
detecting a signal from the region of interest of the subject's brain.

6. The method of claim 1, wherein the machine learning model comprises a contrastive convolutional network.

7. The method of claim 1, wherein the machine learning model comprises a decision tree model.

8. The method of claim 1, wherein generating, using the CBFV measurement and the ABP measurement, the input to the machine learning model comprises:
determining, using the CBFV measurement, a mean CBFV value as an input; and
determining, using the ABP measurement, a mean ABP value as an input.

9. The method of claim 1, wherein:
the CBFV measurement comprises a time series of CBFV values;
the ABP measurement comprises a time series of ABP values; and
generating, using the CBFV measurement and the ABP measurement, the input to the machine learning model comprises:
identifying one or more characteristics of the time series of CBFV values and/or one or more characteristics of the time series of the ABP values; and
generating, using the one or more characteristics of the time series of CBFV values and/or the one or more characteristics of the time series of ABP values, the input to the machine learning model.

10. The method of claim 1, wherein generating, using the CBFV measurement and the ABP measurement, the input to the learning model comprises:
determining frequency domain CBFV values using the CBFV measurement;
determining frequency domain ABP values using the ABP measurement;
determining a mean CBFV value using the frequency domain CBFV values;
determining a mean ABP value using the frequency domain ABP values; and
generating the input using the mean CBFV value and the mean ABP value.

11. The method of claim 1, wherein the machine learning model includes a model based on a resistor capacitor (RC) circuit model of the subject's brain.

12. The method of claim 1, wherein the ICP measurement of the subject's brain is a mean ICP value.

13. The method of claim 1, wherein the ICP measurement of the subject's brain is a time series of ICP values.

14. An ICP measurement system comprising:
one or more probes configured to obtain acoustic measurement data by detecting acoustic signals in a subject's brain; and
at least one computer hardware processor configured to:
determine a cerebral blood flow velocity (CBFV) measurement of the subject's brain using the acoustic measurement data;
obtain an arterial blood pressure (ABP) measurement of the subject's brain;
generate, using the CBFV measurement and the ABP measurement, input to a machine learning model trained to output an ICP measurement; and
provide the input to the machine learning model to obtain an ICP measurement of the subject's brain.

15. At least one non-transitory computer-readable storage medium storing instructions that, when executed by at least one computer hardware processor, cause the at least one computer hardware processor to perform:
obtaining acoustic measurement data obtained from measuring acoustic signals from the subject's brain;
determining a cerebral blood flow velocity (CBFV) measurement of the subject's brain using the acoustic measurement data;
obtaining an arterial blood pressure (ABP) measurement of the subject's brain;
generating, using the CBFV measurement and the ABP measurement, input to a machine learning model trained to output an ICP measurement; and
providing the input to the machine learning model to obtain an ICP measurement of the subject's brain.
